(19) United States
(12) Patent Application Publication
(10) Pub. No.: US 2017/0157135 A1
(43) Pub. Date: Jun. 8, 2017

(54) FUNCTIONALISED AND SUBSTITUTED INDOLES AS ANTI-CANCER AGENTS

(71) Applicant: Novogen Limited, Hornsby, NSW (AU)

(72) Inventors: Ian James, Hornsby (AU); Ian Dixon, Hornsby (AU); Xian Bu, Parkville (AU)

(21) Appl. No.: 15/039,013

(22) PCT Filed: Nov. 25, 2014
(86) PCT No.: PCT/AU2014/050373

§ 371 Date: May 24, 2016

Related U.S. Application Data
(60) Provisional application No. 62/024,571, filed on Jul. 15, 2014.
(60) Provisional application No. 61/908,287, filed on Nov. 25, 2013.

(51) Int. Cl. A61K 31/5377; C07D 403/06; C07D 403/12; C07D 403/14; A61K 31/454; C07D 401/12; C07D 413/14; A61K 31/496; A61K 31/4045; A61K 31/4439; C07D 209/24; C07D 407/14

(52) U.S. Cl. CPC A61K 31/5377; C07D 209/24; C07D 403/06; C07D 403/12; C07D 403/14; C07D 407/14; C07D 401/12; C07D 413/14; A61K 31/496; A61K 31/4045; A61K 31/4439; A61K 31/454

(57) ABSTRACT

The present invention relates to anti-tropomyosin compounds, processes for their preparation, and methods for treating or preventing a proliferative disease, preferably cancer, using compounds of the invention.

### FIELD OF THE INVENTION

The present invention relates broadly to pharmaceutical agents as treatments for proliferative disease such as cancer and a range of degenerative diseases such as osteoarthritis, atherosclerosis, heart disease and inflammatory bowel disease. In particular, the present invention relates to pharmaceutical agents which comprise aryl and/or alkyl substituted indole compounds. The invention further relates to methods for treating or preventing a proliferative disease, preferably cancer. The invention also relates to processes for preparing the compounds.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Cancer kills many thousands of people and is the second largest cause of death in the USA. There have been significant breakthroughs made in treating or preventing a wide variety of cancers. For example patients with breast cancer have benefited from early screening programs as well as a variety of surgical techniques. However, these often prove physically and emotionally debilitating. Moreover, patients who have undergone surgery and subsequent chemotherapy often experience a recurrence in their disease.

A potential new method of specifically attacking cancer cells is through disruption of cancer cells cellular skeletal system comprised predominantly of actin. The actin cytoskeleton is intimately involved in cell division and cell migration. However, actin plays a ubiquitous role as the cytoskeleton of tumor cells and the actin filaments of the muscle sarcomere. The differing roles but similarity in structure make actin a hard target for drug development, due to unwanted off-target side effects.

### SUMMARY OF THE INVENTION

The invention seeks to address one or more of the above mentioned problems, and/or to provide improvements in cancer therapy and in one embodiment provides an anti-tropomyosin compound.

In a first aspect of the invention there is provided a compound of general formula (I), or a pharmaceutically acceptable drug or prodrug thereof, wherein:

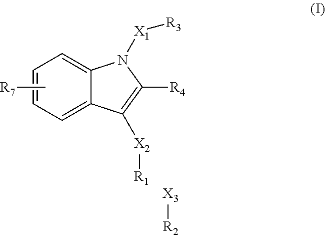

R₁═(CH₂)₀₋₅,

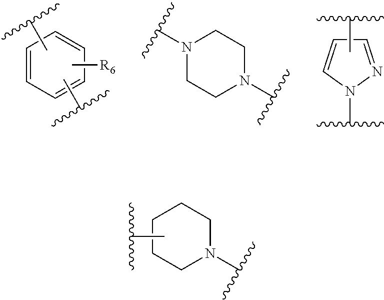

X₁═(CH₂)₀₋₅
X₂ and X₃═O, NH, NHR₅, C(O), C(O)NH, (CH₂)₀₋₅, C(R₅)C(R_{4′})C(O), C(R₅)C(R_{4′})C(O)NH, pyrazole, isooxazole, (R_{4′})pyrimidine
X₄═H, O, NH, NR₅
R₂═CH₃,

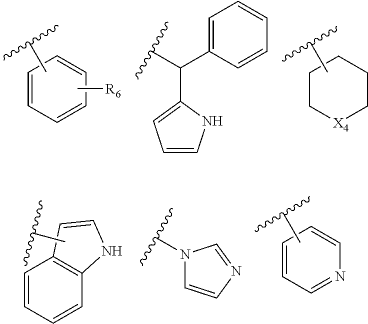

R₄ and R_{4′}═H, CH₃
10 R₅═H, CH₃, (CH₂)₁₋₅CH₃, (CH₂)₁₋₅OCH₃, CF₃, CN, OCF₃
R₆═H, alkyl, halo, alkoxy, amino, aminoalkyl, diaminoalkyl, or a dioxolane ring fused to 2 adjacent carbon atoms of R₁ or R₂
R₇═H, alkyl, alkoxy
R₃═NH₂, N(R₅)₂,

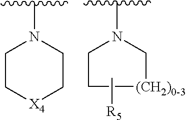

In one embodiment, X₁ is (CH₂)₃. In one embodiment, R₃ is N(R₅)₂. In one embodiment, R₅ is CH₃. In one embodiment, R₅ is CH₂CH₃.

In one embodiment, R₃ is

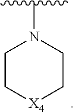

In one embodiment, X₄ is NR₅. In one embodiment, R₅ is CH₃. In one embodiment, X₄ is O.

In one embodiment, R₄ is CH₃.

In one embodiment, X₂ is C(R₅)C(R_{4′})C(O), (R_{4′}) pyrimidine, C(O), or C(R₅)C(R_{4′})C(O)NH.

In one embodiment, X₂ is C(R₅)C(R_{4′})C(O), R₅ is H and R_{4′} is H. In one embodiment, X₂ is C(R₅)C(R_{4′})C(O), R₅ is H and R_{4′} is CH₃.

In one embodiment, X₂ is (R_{4′}) pyrimidine and R_{4′} is CH₃.

In one embodiment, X₂ is C(R₅)C(R_{4′})C(O)NH, R₅ is H and R_{4′} is H.

In one embodiment, R₁ is CH₂,

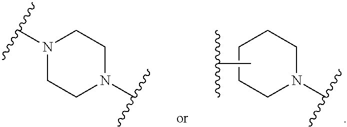

In one embodiment,

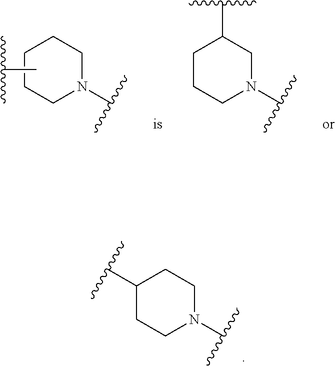

In one embodiment, X₃ is CH₂, (CH₂)₂, (CH₂)₃ or C(O).

In one embodiment, R₂ is

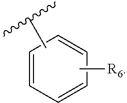

In one embodiment, R₆ is H, alkoxy, halo or the dioxolane ring. In one embodiment, alkoxy is OCH₃. In one embodiment, halo is F.

In one embodiment, R₂ is CH₃.

In one embodiment, R₂ is

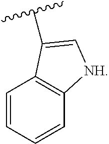 [SMILES: *c1c[nH]c2ccccc12]

In one embodiment, R₂ is

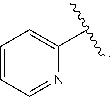 [SMILES: *c1ccccn1]

In one embodiment, R₇ is H.

In one embodiment, R₇ is alkoxy. In one embodiment, alkoxy is OCH₂CH₃ or OCH₃.

Preferably, the compounds of the first aspect of the invention are exemplified in the following structures:

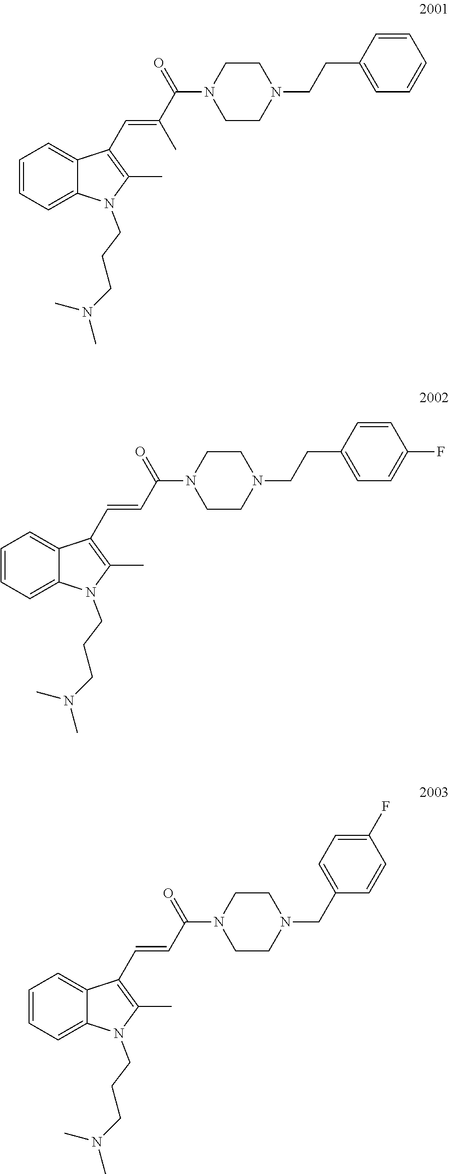 [SMILES: C/C(=C\c1c(C)n(CCCN(C)C)c2ccccc12)C(=O)N1CCN(CCc2ccccc2)CC1 ; Cc1c(/C=C/C(=O)N2CCN(CCc3ccc(F)cc3)CC2)c2ccccc2n1CCCN(C)C ; Cc1c(/C=C/C(=O)N2CCN(Cc3ccc(F)cc3)CC2)c2ccccc2n1CCCN(C)C]

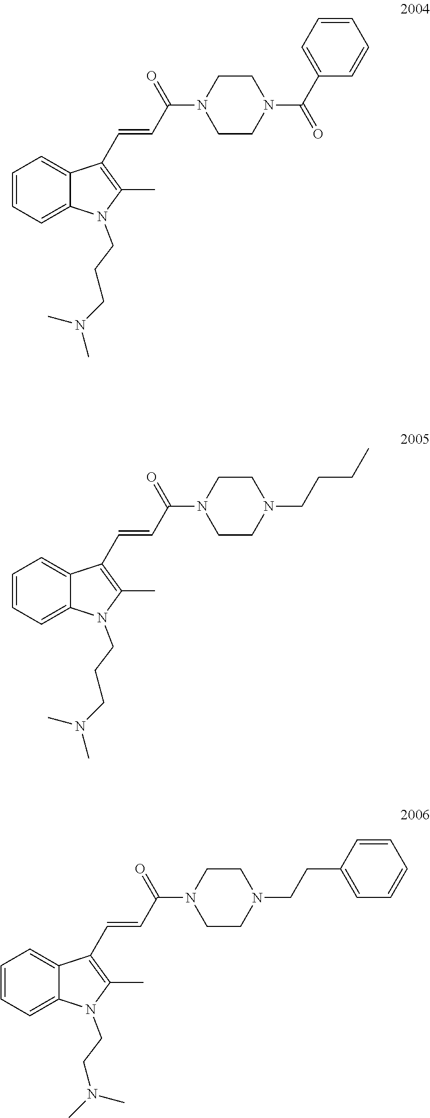 [SMILES: Cc1c(/C=C/C(=O)N2CCN(C(=O)c3ccccc3)CC2)c2ccccc2n1CCCN(C)C ; CCCCN1CCN(C(=O)/C=C/c2c(C)n(CCCN(C)C)c3ccccc23)CC1 ; Cc1c(/C=C/C(=O)N2CCN(CCc3ccccc3)CC2)c2ccccc2n1CCN(C)C]

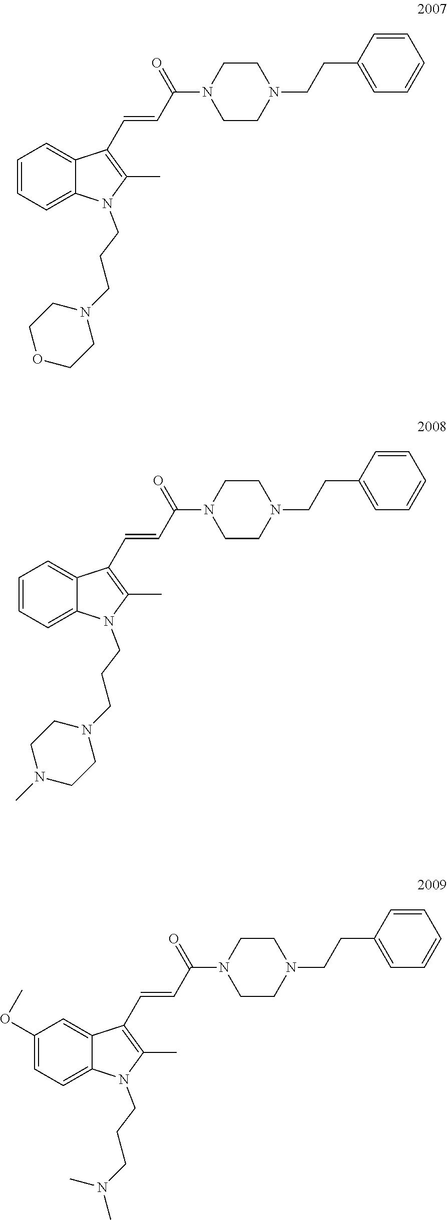 [SMILES: Cc1c(/C=C/C(=O)N2CCN(CCc3ccccc3)CC2)c2ccccc2n1CCCN1CCOCC1 ; Cc1c(/C=C/C(=O)N2CCN(CCc3ccccc3)CC2)c2ccccc2n1CCCN1CCN(C)CC1 ; COc1ccc2c(c1)c(/C=C/C(=O)N1CCN(CCc3ccccc3)CC1)c(C)n2CCCN(C)C]

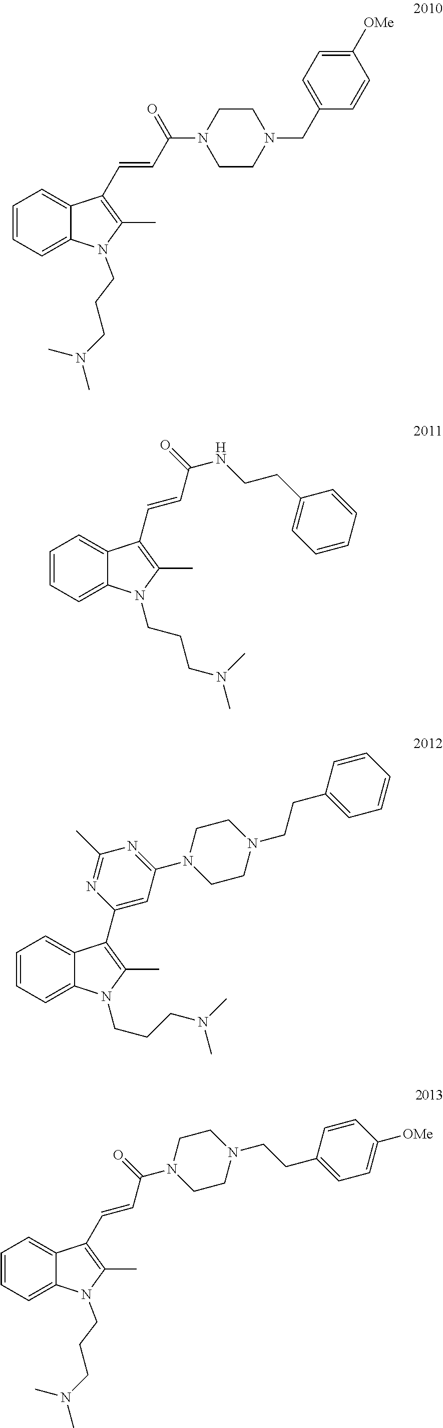 [SMILES: COc1ccc(CN2CCN(C(=O)/C=C/c3c(C)n(CCCN(C)C)c4ccccc34)CC2)cc1 ; Cc1c(/C=C/C(=O)NCCc2ccccc2)c2ccccc2n1CCCN(C)C ; Cc1nc(-c2c(C)n(CCCN(C)C)c3ccccc23)cc(N2CCN(CCc3ccccc3)CC2)n1 ; COc1ccc(CCN2CCN(C(=O)/C=C/c3c(C)n(CCCN(C)C)c4ccccc34)CC2)cc1]

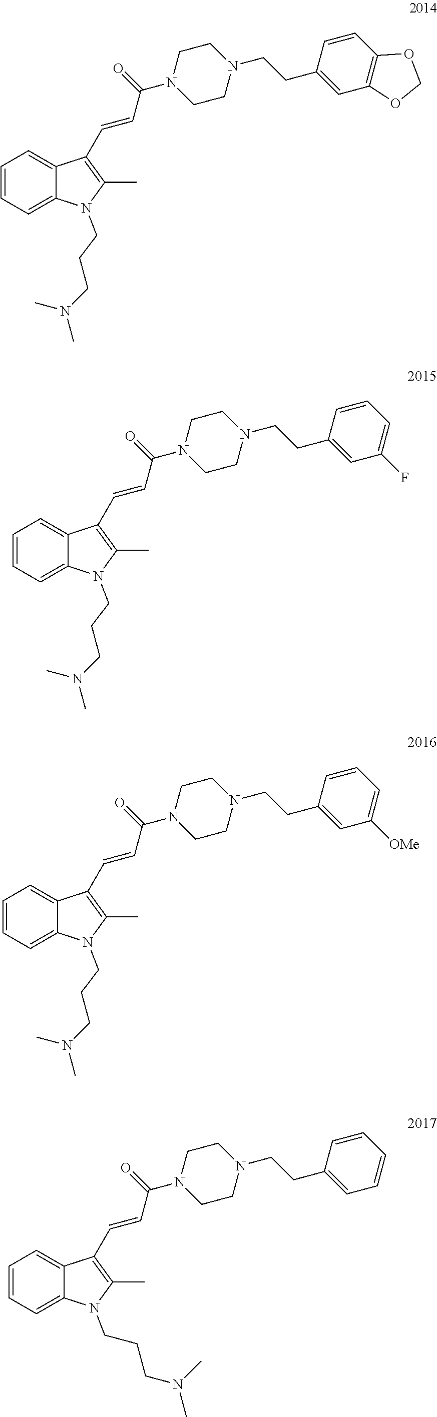 [SMILES: Cc1c(/C=C/C(=O)N2CCN(CCc3ccc4c(c3)OCO4)CC2)c2ccccc2n1CCCN(C)C ; Cc1c(/C=C/C(=O)N2CCN(CCc3cccc(F)c3)CC2)c2ccccc2n1CCCN(C)C ; COc1cccc(CCN2CCN(C(=O)/C=C/c3c(C)n(CCCN(C)C)c4ccccc34)CC2)c1 ; Cc1c(/C=C/C(=O)N2CCN(CCc3ccccc3)CC2)c2ccccc2n1CCCN(C)C]

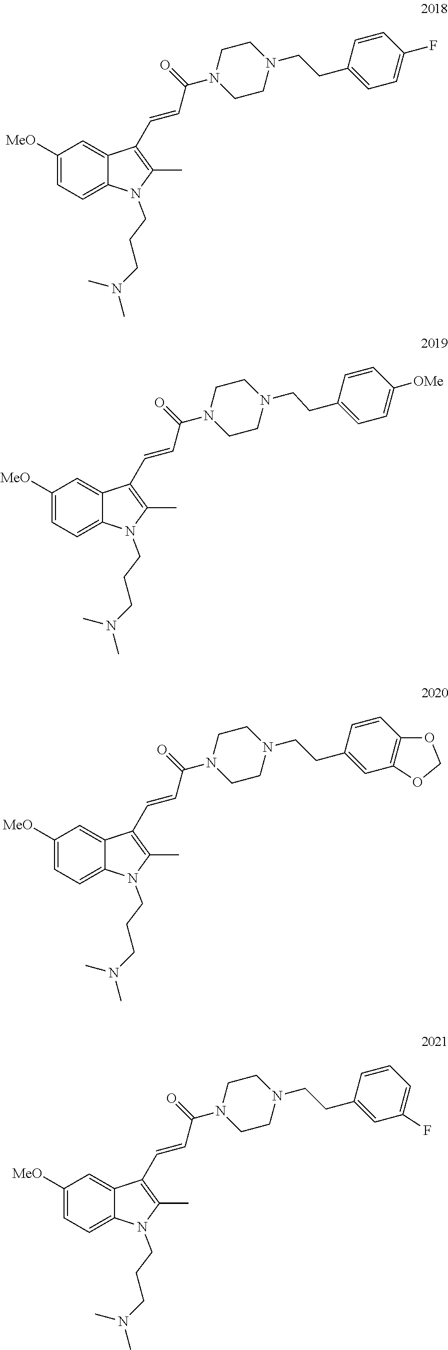 [SMILES: COc1ccc2c(c1)c(/C=C/C(=O)N1CCN(CCc3ccc(F)cc3)CC1)c(C)n2CCCN(C)C ; COc1ccc(CCN2CCN(C(=O)/C=C/c3c(C)n(CCCN(C)C)c4ccc(OC)cc34)CC2)cc1 ; COc1ccc2c(c1)c(/C=C/C(=O)N1CCN(CCc3ccc4c(c3)OCO4)CC1)c(C)n2CCCN(C)C ; COc1ccc2c(c1)c(/C=C/C(=O)N1CCN(CCc3cccc(F)c3)CC1)c(C)n2CCCN(C)C]

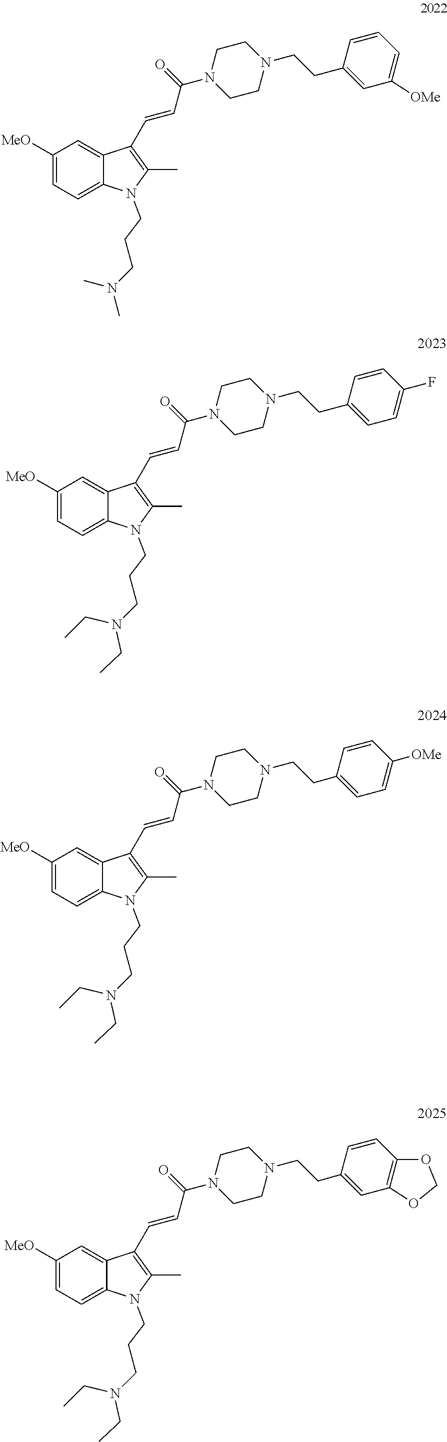 [SMILES: COc1cccc(CCN2CCN(C(=O)/C=C/c3c(C)n(CCCN(C)C)c4ccc(OC)cc34)CC2)c1 ; CCN(CC)CCCn1c(C)c(/C=C/C(=O)N2CCN(CCc3ccc(F)cc3)CC2)c2cc(OC)ccc21 ; CCN(CC)CCCn1c(C)c(/C=C/C(=O)N2CCN(CCc3ccc(OC)cc3)CC2)c2cc(OC)ccc21 ; CCN(CC)CCCn1c(C)c(/C=C/C(=O)N2CCN(CCc3ccc4c(c3)OCO4)CC2)c2cc(OC)ccc21]

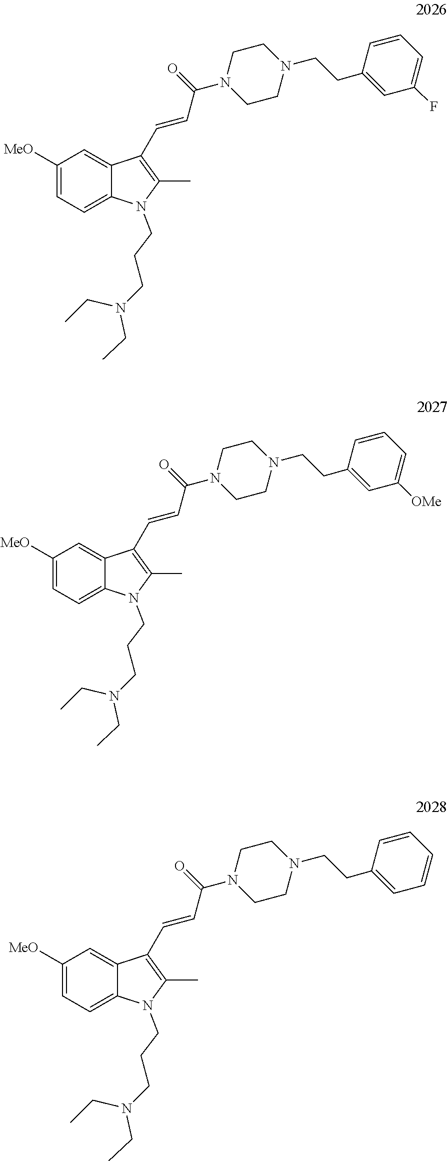 [SMILES: CCN(CC)CCCn1c(C)c(/C=C/C(=O)N2CCN(CCc3cccc(F)c3)CC2)c2cc(OC)ccc21 ; CCN(CC)CCCn1c(C)c(/C=C/C(=O)N2CCN(CCc3cccc(OC)c3)CC2)c2cc(OC)ccc21 ; CCN(CC)CCCn1c(C)c(/C=C/C(=O)N2CCN(CCc3ccccc3)CC2)c2cc(OC)ccc21]

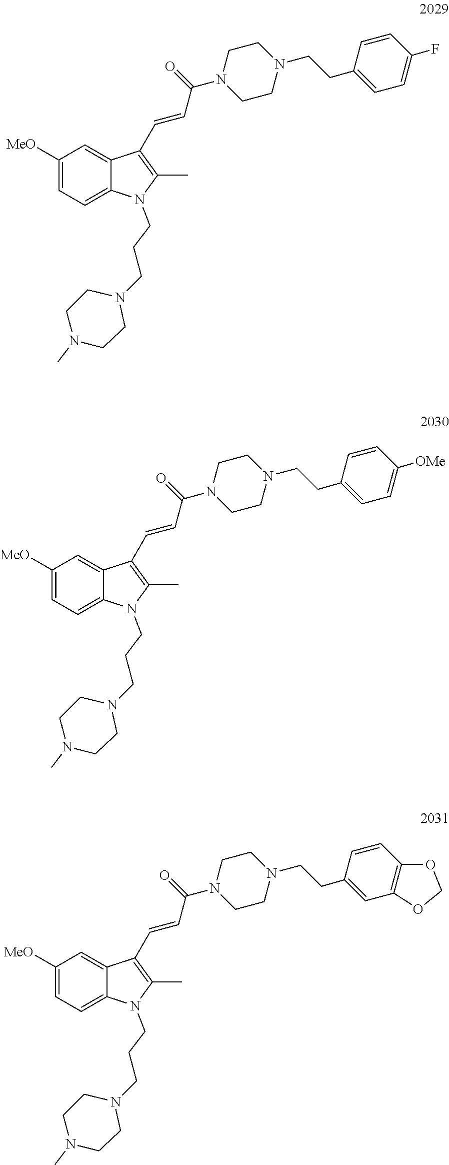 [SMILES: COc1ccc2c(c1)c(/C=C/C(=O)N1CCN(CCc3ccc(F)cc3)CC1)c(C)n2CCCN1CCN(C)CC1 ; COc1ccc(CCN2CCN(C(=O)/C=C/c3c(C)n(CCCN4CCN(C)CC4)c4ccc(OC)cc34)CC2)cc1 ; COc1ccc2c(c1)c(/C=C/C(=O)N1CCN(CCc3ccc4c(c3)OCO4)CC1)c(C)n2CCCN1CCN(C)CC1]

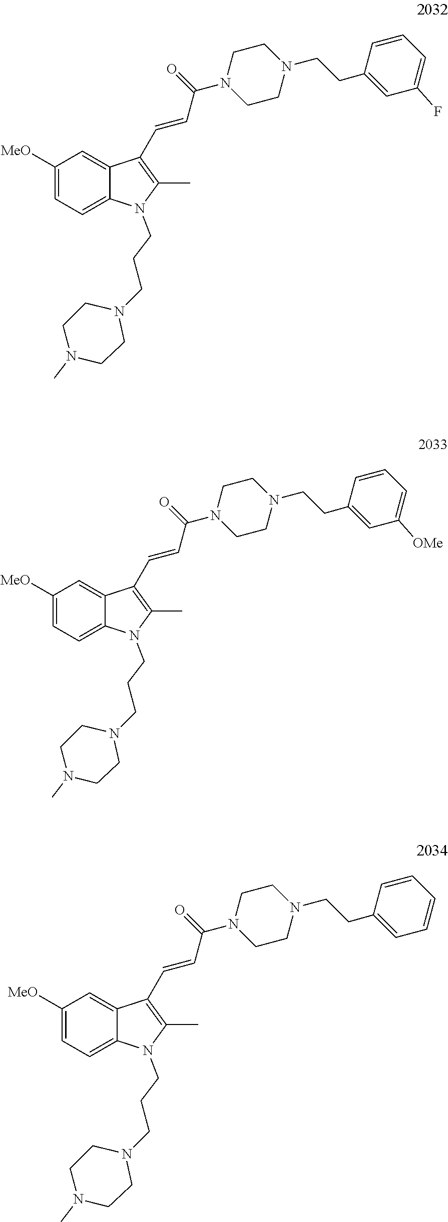 [SMILES: COc1ccc2c(c1)c(/C=C/C(=O)N1CCN(CCc3cccc(F)c3)CC1)c(C)n2CCCN1CCN(C)CC1 ; COc1cccc(CCN2CCN(C(=O)/C=C/c3c(C)n(CCCN4CCN(C)CC4)c4ccc(OC)cc34)CC2)c1 ; COc1ccc2c(c1)c(/C=C/C(=O)N1CCN(CCc3ccccc3)CC1)c(C)n2CCCN1CCN(C)CC1]

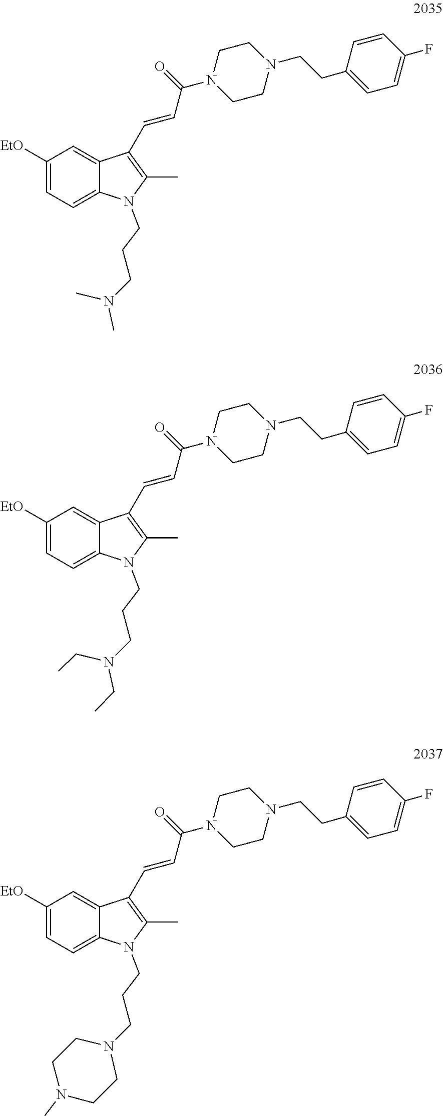 [SMILES: CCOc1ccc2c(c1)c(/C=C/C(=O)N1CCN(CCc3ccc(F)cc3)CC1)c(C)n2CCCN(C)C ; CCOc1ccc2c(c1)c(/C=C/C(=O)N1CCN(CCc3ccc(F)cc3)CC1)c(C)n2CCCN(CC)CC ; CCOc1ccc2c(c1)c(/C=C/C(=O)N1CCN(CCc3ccc(F)cc3)CC1)c(C)n2CCCN1CCN(C)CC1]

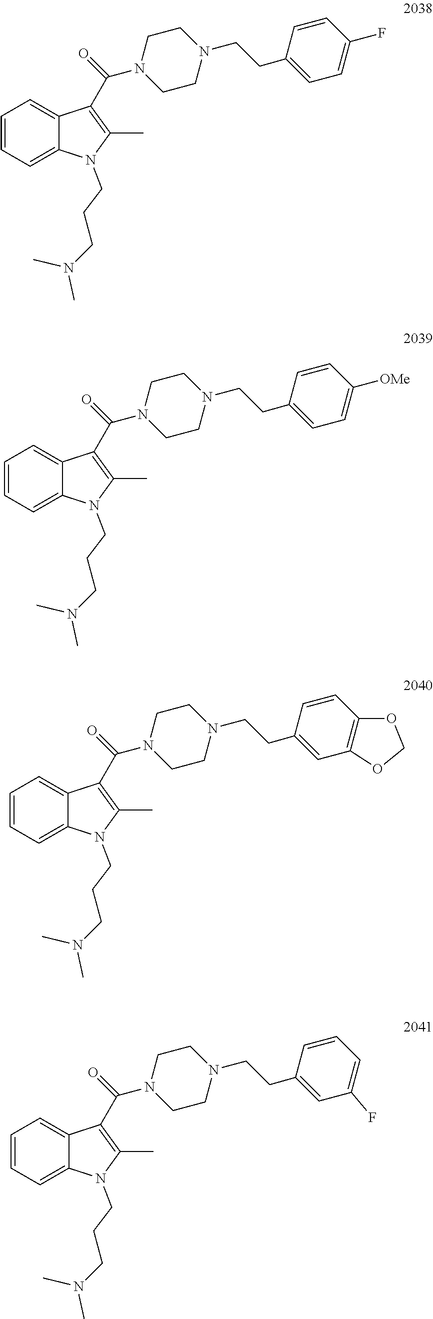 [SMILES: Cc1c(C(=O)N2CCN(CCc3ccc(F)cc3)CC2)c2ccccc2n1CCCN(C)C ; COc1ccc(CCN2CCN(C(=O)c3c(C)n(CCCN(C)C)c4ccccc34)CC2)cc1 ; Cc1c(C(=O)N2CCN(CCc3ccc4c(c3)OCO4)CC2)c2ccccc2n1CCCN(C)C ; Cc1c(C(=O)N2CCN(CCc3cccc(F)c3)CC2)c2ccccc2n1CCCN(C)C]

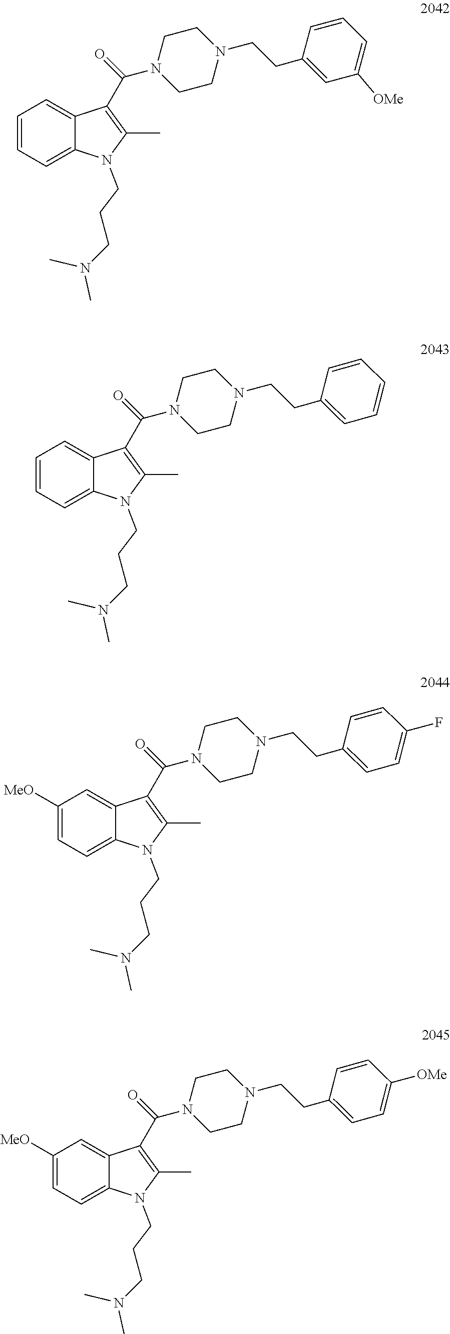 [SMILES: COc1cccc(CCN2CCN(C(=O)c3c(C)n(CCCN(C)C)c4ccccc34)CC2)c1 ; Cc1c(C(=O)N2CCN(CCc3ccccc3)CC2)c2ccccc2n1CCCN(C)C ; COc1ccc2c(c1)c(C(=O)N1CCN(CCc3ccc(F)cc3)CC1)c(C)n2CCCN(C)C ; COc1ccc(CCN2CCN(C(=O)c3c(C)n(CCCN(C)C)c4ccc(OC)cc34)CC2)cc1]

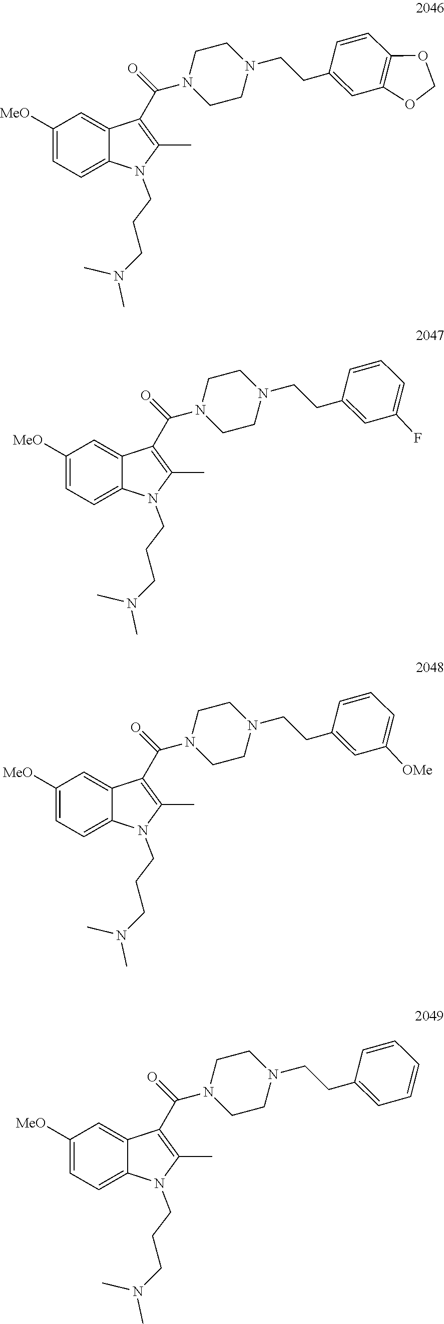 [SMILES: COc1ccc2c(c1)c(C(=O)N1CCN(CCc3ccc4c(c3)OCO4)CC1)c(C)n2CCCN(C)C ; COc1ccc2c(c1)c(C(=O)N1CCN(CCc3cccc(F)c3)CC1)c(C)n2CCCN(C)C ; COc1cccc(CCN2CCN(C(=O)c3c(C)n(CCCN(C)C)c4ccc(OC)cc34)CC2)c1 ; COc1ccc2c(c1)c(C(=O)N1CCN(CCc3ccccc3)CC1)c(C)n2CCCN(C)C]

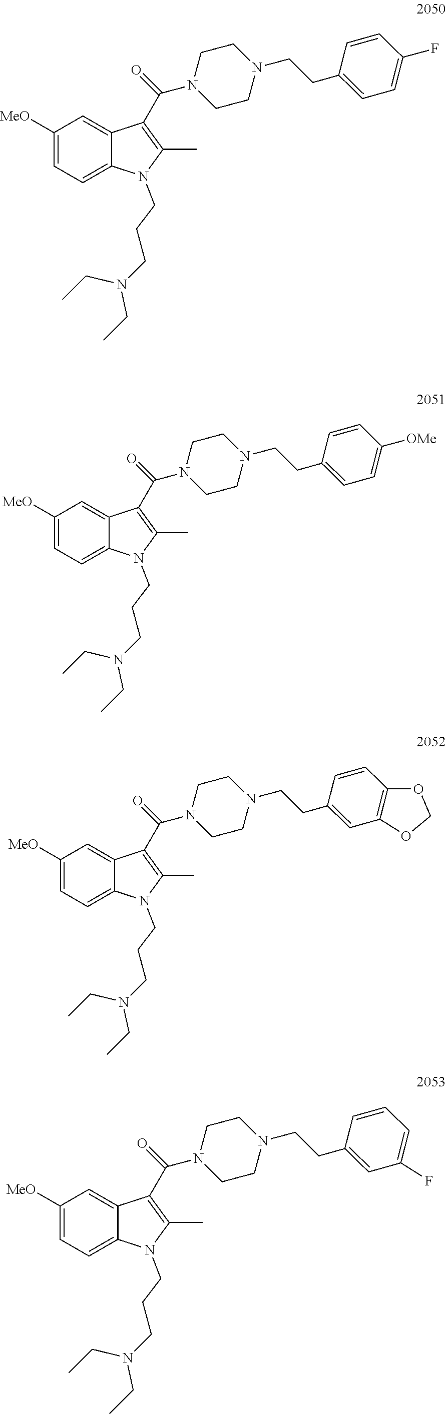 [SMILES: CCN(CC)CCCn1c(C)c(C(=O)N2CCN(CCc3ccc(F)cc3)CC2)c2cc(OC)ccc21 ; CCN(CC)CCCn1c(C)c(C(=O)N2CCN(CCc3ccc(OC)cc3)CC2)c2cc(OC)ccc21 ; CCN(CC)CCCn1c(C)c(C(=O)N2CCN(CCc3ccc4c(c3)OCO4)CC2)c2cc(OC)ccc21 ; CCN(CC)CCCn1c(C)c(C(=O)N2CCN(CCc3cccc(F)c3)CC2)c2cc(OC)ccc21]

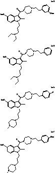 [SMILES: CCN(CC)CCCn1c(C)c(C(=O)N2CCN(CCc3cccc(OC)c3)CC2)c2cc(OC)ccc21 ; CCN(CC)CCCn1c(C)c(C(=O)N2CCN(CCc3ccccc3)CC2)c2cc(OC)ccc21 ; COc1ccc2c(c1)c(C(=O)N1CCN(CCc3ccc(F)cc3)CC1)c(C)n2CCCN1CCN(C)CC1 ; COc1ccc(CCN2CCN(C(=O)c3c(C)n(CCCN4CCN(C)CC4)c4ccc(OC)cc34)CC2)cc1]

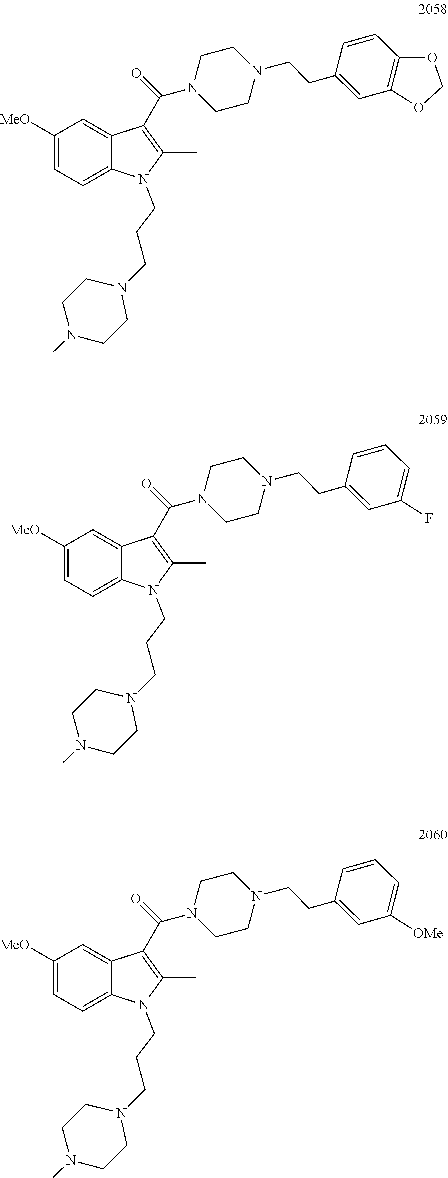 [SMILES: COc1ccc2c(c1)c(C(=O)N1CCN(CCc3ccc4c(c3)OCO4)CC1)c(C)n2CCCN1CCN(C)CC1 ; COc1ccc2c(c1)c(C(=O)N1CCN(CCc3cccc(F)c3)CC1)c(C)n2CCCN1CCN(C)CC1 ; COc1cccc(CCN2CCN(C(=O)c3c(C)n(CCCN4CCN(C)CC4)c4ccc(OC)cc34)CC2)c1]

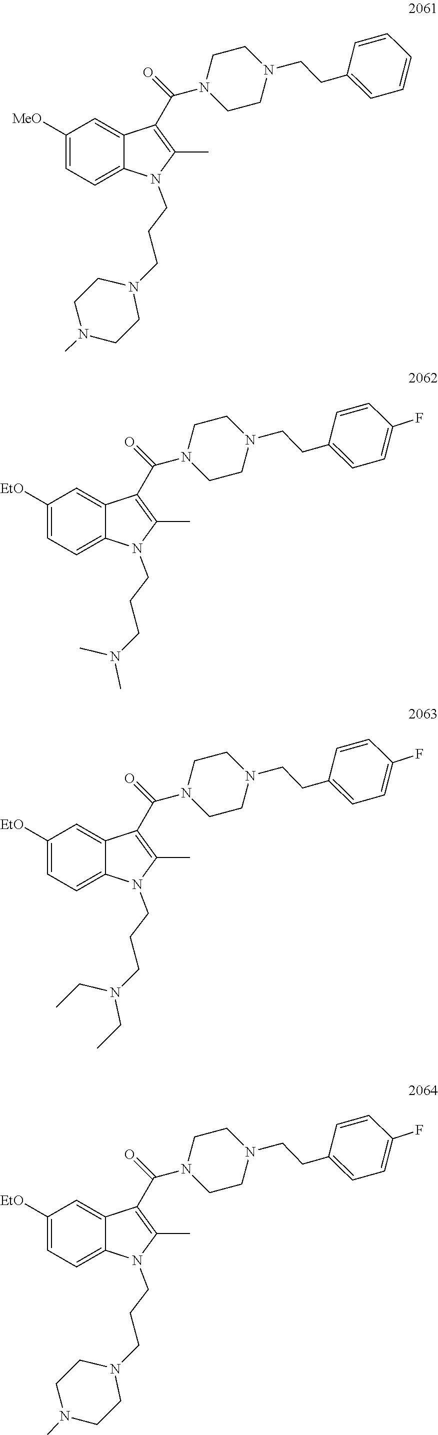 [SMILES: COc1ccc2c(c1)c(C(=O)N1CCN(CCc3ccccc3)CC1)c(C)n2CCCN1CCN(C)CC1 ; CCOc1ccc2c(c1)c(C(=O)N1CCN(CCc3ccc(F)cc3)CC1)c(C)n2CCCN(C)C ; CCOc1ccc2c(c1)c(C(=O)N1CCN(CCc3ccc(F)cc3)CC1)c(C)n2CCCN(CC)CC ; CCOc1ccc2c(c1)c(C(=O)N1CCN(CCc3ccc(F)cc3)CC1)c(C)n2CCCN1CCN(C)CC1]

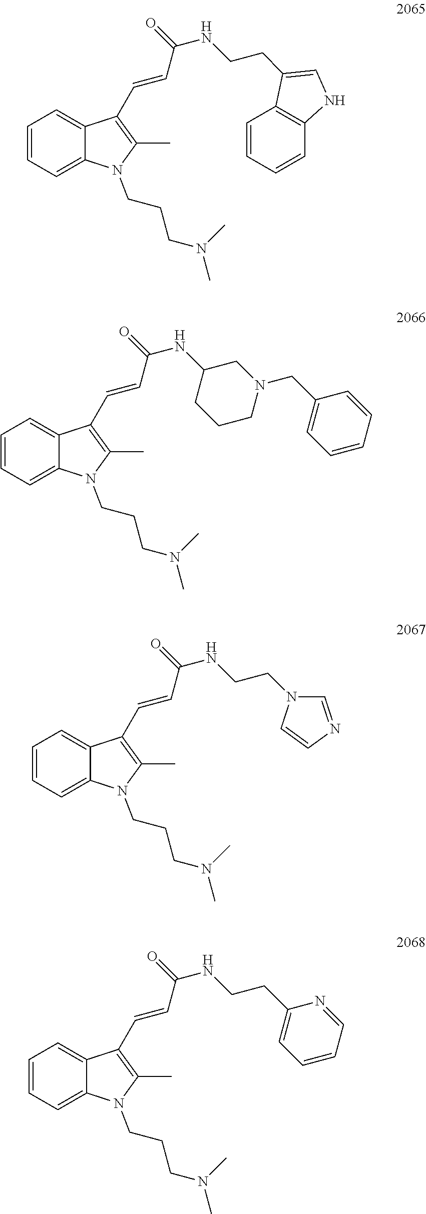 [SMILES: Cc1c(/C=C/C(=O)NCCc2c[nH]c3ccccc23)c2ccccc2n1CCCN(C)C ; Cc1c(/C=C/C(=O)NC2CCCN(Cc3ccccc3)C2)c2ccccc2n1CCCN(C)C ; Cc1c(/C=C/C(=O)NCCn2ccnc2)c2ccccc2n1CCCN(C)C ; Cc1c(/C=C/C(=O)NCCc2ccccn2)c2ccccc2n1CCCN(C)C]

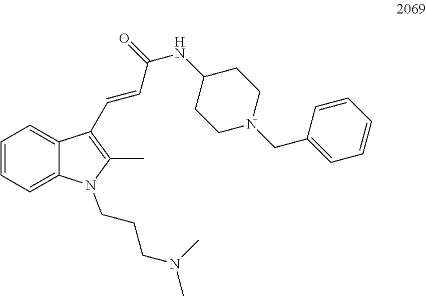 [SMILES: Cc1c(/C=C/C(=O)NC2CCN(Cc3ccccc3)CC2)c2ccccc2n1CCCN(C)C]

In one embodiment, the compounds are:
(E)-3-(1-(3-(dimethylamino)propyl)-2-methyl-1H-indol-3-yl)-2-methyl-1-(4-phenethylpiperazin-1-yl)prop-2-en-1-one
(E)-3-(1-(3-(dimethylamino)propyl)-2-methyl-1H-indol-3-yl)-1-(4-(4-fluorophenethyl)piperazin-1-yl)prop-2-en-1-one
(E)-3-(1-(3-(dimethylamino)propyl)-2-methyl-1H-indol-3-yl)-1-(4-(4-fluorobenzyl)piperazin-1-yl)prop-2-en-1-one
(E)-1-(4-benzoylpiperazin-1-yl)-3-(1-(3-(dimethylamino)propyl)-2-methyl-1H-indol-3-yl)prop-2-en-1-one
(E)-1-(4-butylpiperazin-1-yl)-3-(1-(3-(dimethylamino)propyl)-2-methyl-1H-indol-3-yl)prop-2-en-1-one
(E)-3-(1-(2-(dimethylamino)ethyl)-2-methyl-1H-indol-3-yl)-1-(4-phenethylpiperazin-1-yl)prop-2-en-1-one
(E)-3-(2-methyl-1-(3-morpholinopropyl)-1H-indol-3-yl)-1-(4-phenethylpiperazin-1-yl)prop-2-en-1-one
(E)-3-(2-methyl-1-(3-(4-methylpiperazin-1-yl)propyl)-1H-indol-3-yl)-1-(4-phenethylpiperazin-1-yl)prop-2-en-1-one
(E)-3-(1-(3-(dimethylamino)propyl)-5-methoxy-2-methyl-1H-indol-3-yl)-1-(4-phenethylpiperazin-1-yl)prop-2-en-1-one
(E)-3-(1-(3-(dimethylamino)propyl)-2-methyl-1H-indol-3-yl)-1-(4-(4-methoxybenzyl)piperazin-1-yl)prop-2-en-1-one
(E)-3-(1-(3-(dimethylamino)propyl)-2-methyl-1H-indol-3-yl)-N-phenethylacrylamide
N,N-dimethyl-3-(3-(2-methyl-6-(4-phenethylpiperazin-1-yl)pyrimidin-4-yl)-1H-indol-1-yl)propan-1-amine
(E)-3-(1-(3-(dimethylamino)propyl)-2-methyl-1H-indol-3-yl)-1-(4-(4-methoxyphenethyl)piperazin-1-yl)prop-2-en-1-one
(E)-1-(4-(2-(benzo[d][1,3]dioxol-5-yl)ethyl)piperazin-1-yl)-3-(1-(3-(dimethylamino)propyl)-2-methyl-1H-indol-3-yl)prop-2-en-1-one
(E)-3-(1-(3-(dimethylamino)propyl)-2-methyl-1H-indol-3-yl)-1-(4-(3-fluorophenethyl)piperazin-1-yl)prop-2-en-1-one
(E)-3-(1-(3-(dimethylamino)propyl)-2-methyl-1H-indol-3-yl)-1-(4-(3-methoxyphenethyl)piperazin-1-yl)prop-2-en-1-one
(E)-3-(1-(3-(dimethylamino)propyl)-2-methyl-1H-indol-3-yl)-1-(4-phenethylpiperazin-1-yl)prop-2-en-1-one
(E)-3-(1-(3-(dimethyl amino)propyl)-5-methoxy-2-methyl-1H-indol-3-yl)-1-(4-(4-fluorophenethyl)piperazin-1-yl)prop-2-en-1-one
(E)-3-(1-(3-(dimethylamino)propyl)-5-methoxy-2-methyl-1H-indol-3-yl)-1-(4-(4-methoxyphenethyl)piperazin-1-yl)prop-2-en-1-one
(E)-1-(4-(2-(benzo[d][1,3]dioxol-5-yl)ethyl)piperazin-1-yl)-3-(1-(3-(dimethylamino)propyl)-5-methoxy-2-methyl-1H-indol-3-yl)prop-2-en-1-one
(E)-3-(1-(3-(dimethylamino)propyl)-5-methoxy-2-methyl-1H-indol-3-yl)-1-(4-(3-fluorophenethyl)piperazin-1-yl)prop-2-en-1-one
(E)-3-(1-(3-(dimethylamino)propyl)-5-methoxy-2-methyl-1H-indol-3-yl)-1-(4-(3-methoxyphenethyl)piperazin-1-yl)prop-2-en-1-one
(E)-3-(1-(3-(diethylamino)propyl)-5-methoxy-2-methyl-1H-indol-3-yl)-1-(4-(4-fluorophenethyl)piperazin-1-yl)prop-2-en-1-one
(E)-3-(1-(3-(diethylamino)propyl)-5-methoxy-2-methyl-1H-indol-3-yl)-1-(4-(4-methoxyphenethyl)piperazin-1-yl)prop-2-en-1-one
(E)-1-(4-(2-(benzo[d][1,3]dioxol-5-yl)ethyl)piperazin-1-yl)-3-(1-(3-(diethylamino)propyl)-5-methoxy-2-methyl-1H-indol-3-yl)prop-2-en-1-one
(E)-3-(1-(3-(diethylamino)propyl)-5-methoxy-2-methyl-1H-indol-3-yl)-1-(4-(3-fluorophenethyl)piperazin-1-yl)prop-2-en-1-one
(E)-3-(1-(3-(diethylamino)propyl)-5-methoxy-2-methyl-1H-indol-3-yl)-1-(4-(3-methoxyphenethyl)piperazin-1-yl)prop-2-en-1-one
(E)-3-(1-(3-(diethylamino)propyl)-5-methoxy-2-methyl-1H-indol-3-yl)-1-(4-phenethylpiperazin-1-yl)prop-2-en-1-one
(E)-1-(4-(4-fluorophenethyl)piperazin-1-yl)-3-(5-methoxy-2-methyl-1-(3-(4-methylpiperazin-1-yl)propyl)-1H-indol-3-yl)prop-2-en-1-one
(E)-3-(5-methoxy-2-methyl-1-(3-(4-methylpiperazin-1-yl)propyl)-1H-indol-3-yl)-1-(4-(4-methoxyphenethyl)piperazin-1-yl)prop-2-en-1-one
(E)-1-(4-(2-(benzo[d][1,3]dioxol-5-yl)ethyl)piperazin-1-yl)-3-(5-methoxy-2-methyl-1-(3-(4-methylpiperazin-1-yl)propyl)-1H-indol-3-yl)prop-2-en-1-one
(E)-1-(4-(3-fluorophenethyl)piperazin-1-yl)-3-(5-methoxy-2-methyl-1-(3-(4-methylpiperazin-1-yl)propyl)-1H-indol-3-yl)prop-2-en-1-one
(E)-3-(5-methoxy-2-methyl-1-(3-(4-methylpiperazin-1-yl)propyl)-1H-indol-3-yl)-1-(4-(3-methoxyphenethyl)piperazin-1-yl)prop-2-en-1-one
(E)-3-(5-methoxy-2-methyl-1-(3-(4-methylpiperazin-1-yl)propyl)-1H-indol-3-yl)-1-(4-phenethylpiperazin-1-yl)prop-2-en-1-one
(E)-3-(1-(3-(dimethylamino)propyl)-5-ethoxy-2-methyl-1H-indol-3-yl)-1-(4-(4-fluorophenethyl)piperazin-1-yl)prop-2-en-1-one
(E)-3-(1-(3-(diethylamino)propyl)-5-ethoxy-2-methyl-1H-indol-3-yl)-1-(4-(4-fluorophenethyl)piperazin-1-yl)prop-2-en-1-one
(E)-3-(5-ethoxy-2-methyl-1-(3-(4-methylpiperazin-1-yl)propyl)-1H-indol-3-yl)-1-(4-(4-fluorophenethyl)piperazin-1-yl)prop-2-en-1-one
(1-(3-(dimethylamino)propyl)-2-methyl-1H-indol-3-yl)(4-(4-fluorophenethyl) piperazin-1-yl)methanone
(1-(3-(dimethylamino)propyl)-2-methyl-1H-indol-3-yl)(4-(4-methoxyphenethyl)piperazin-1-yl)methanone
(4-(2-(benzo[d][1,3]dioxol-5-yl)ethyl)piperazin-1-yl)(1-(3-(dimethylamino)propyl)-2-methyl-1H-indol-3-yl)methanone
(1-(3-(dimethylamino)propyl)-2-methyl-1H-indol-3-yl)(4-(3-fluorophenethyl)piperazin-1-yl)methanone
(1-(3-(dimethylamino)propyl)-2-methyl-1H-indol-3-yl)(4-(3-methoxyphenethyl)piperazin-1-yl)methanone
(1-(3-(dimethylamino)propyl)-2-methyl-1H-indol-3-yl)(4-phenethylpiperazin-1-yl)methanone
(1-(3-(dimethylamino)propyl)-5-methoxy-2-methyl-1H-indol-3-yl)(4-(4-fluorophenethyl)piperazin-1-yl)methanone
(1-(3-(dimethylamino)propyl)-5-methoxy-2-methyl-1H-indol-3-yl)(4-(4-methoxyphenethyl)piperazin-1-yl)methanone
(4-(2-(benzo[d][1,3]dioxol-5-yl)ethyl)piperazin-1-yl)(1-(3-(dimethylamino)propyl)-5-methoxy-2-methyl-1H-indol-3-yl) methanone
(1-(3-(dimethylamino)propyl)-5-methoxy-2-methyl-1H-indol-3-yl)(4-(3-fluorophenethyl)piperazin-1-yl)methanone
(1-(3-(dimethylamino)propyl)-5-methoxy-2-methyl-1H-indol-3-yl)(4-(3-methoxyphenethyl)piperazin-1-yl)methanone
(1-(3-(dimethylamino)propyl)-5-methoxy-2-methyl-1H-indol-3-yl)(4-phenethylpiperazin-1-yl)methanone
(1-(3-(diethylamino)propyl)-5-methoxy-2-methyl-1H-indol-3-yl)(4-(4-fluorophenethyl)piperazin-1-yl)methanone
(1-(3-(diethylamino)propyl)-5-methoxy-2-methyl-1H-indol-3-yl)(4-(4-methoxyphenethyl)piperazin-1-yl)methanone
(4-(2-(benzo[d][1,3]dioxol-5-yl)ethyl)piperazin-1-yl)(1-(3-(diethylamino)propyl)-5-methoxy-2-methyl-1H-indol-3-yl)methanone
(1-(3-(diethylamino)propyl)-5-methoxy-2-methyl-1H-indol-3-yl)(4-(3-fluorophenethyl)piperazin-1-yl)methanone
(1-(3-(diethylamino)propyl)-5-meth oxy-2-methyl-1H-indol-3-yl)(4-(3-methoxyphenethyl)piperazin-1-yl)methanone
(1-(3-(diethylamino)propyl)-5-methoxy-2-methyl-1H-indol-3-yl)(4-phenethylpiperazin-1-yl)methanone
(4-(4-fluorophenethyl)piperazin-1-yl)(5-methoxy-2-methyl-1-(3-(4-methylpiperazin-1-yl)propyl)-1H-indol-3-yl)methanone
(5-methoxy-2-methyl-1-(3-(4-methylpiperazin-1-yl)propyl)-1H-indol-3-yl)(4-(4-methoxyphenethyl)piperazin-1-yl)methanone
(4-(2-(benzo[d][1,3]dioxol-5-yl)ethyl)piperazin-1-yl)(5-methoxy-2-methyl-1-(3-(4-methylpiperazin-1-yl)propyl)-1H-indol-3-yl)methanone
(4-(3-fluorophenethyl)piperazin-1-yl)(5-methoxy-2-methyl-1-(3-(4-methylpiperazin-1-yl)propyl)-1H-indol-3-yl)methanone
(5-methoxy-2-methyl-1-(3-(4-methyl piperazin-1-yl)propyl)-1H-indol-3-yl)(4-(3-methoxyphenethyl)piperazin-1-yl)methanone
(5-methoxy-2-methyl-1-(3-(4-methylpiperazin-1-yl)propyl)-1H-indol-3-yl)(4-phenethylpiperazin-1-yl)methanone
(1-(3-(dimethylamino)propyl)-5-ethoxy-2-methyl-1H-indol-3-yl)(4-(4-fluorophenethyl)piperazin-1-yl)methanone
(1-(3-(diethylamino)propyl)-5-ethoxy-2-methyl-1H-indol-3-yl)(4-(4-fluorophenethyl)piperazin-1-yl)methanone
(5-ethoxy-2-methyl-1-(3-(4-methylpiperazin-1-yl)propyl)-1H-indol-3-yl)(4-(4-fluorophenethyl)piperazin-1-yl)methanone
(E)-N-(2-(1H-indol-3-yl)ethyl)-3-(1-(3-(dimethylamino)propyl)-2-methyl-1H-indol-3-yl)acrylamide
(E)-N-(1-benzylpiperidin-3-yl)-3-(1-(3-(dimethylamino)propyl)-2-methyl-1H-indol-3-yl)acrylamide
(E)-N-(2-(1H-imidazol-1-yl)ethyl)-3-(1-(3-(dimethylamino)propyl)-2-methyl-1H-indol-3-yl)acrylamide
(E)-3-(1-(3-(dimethylamino)propyl)-2-methyl-1H-indol-3-yl)-N-(2-(pyridin-2-yl)ethyl)acrylamide
(E)-N-(1-benzylpiperidin-4-yl)-3-(1-(3-(dimethylamino)propyl)-2-methyl-1H-indol-3-yl)acrylamide

In a second aspect the invention relates to a pharmaceutical composition comprising a compound of formula (I) together with a pharmaceutically acceptable carrier, diluent or excipient.

Compounds and pharmaceutical compositions according to the present invention may be suitable for the treatment or prevention of a proliferative disease. Accordingly, in another aspect the invention relates to a method of treating or preventing a proliferative disease in a subject, the method comprising administering to the subject an effective amount of a compound of formula (I) according to the first aspect of the invention or a pharmaceutical composition according to the second aspect of the invention.

In a further aspect, the present invention relates to the use of a compound of formula (I) according to the first aspect of the invention or a pharmaceutical composition according to the second aspect of the invention in the manufacture of a medicament for treating or preventing a proliferative disease.

In a further aspect, the present invention relates to the use of a compound of formula (I) according to the first aspect of the invention or a pharmaceutical composition according to the second aspect of the invention for the treatment or prevention of a proliferative disease in a subject.

In a further aspect, the present invention relates to a compound of formula (I) according to the first aspect of the invention or a pharmaceutical composition according to the second aspect of the invention for use in the treatment or prevention of a proliferative disease in a subject.

In a further aspect, the present invention relates to a pharmaceutical composition for use in the treatment or prevention of a proliferative disease in a subject, in any of the embodiments described in the specification.

In a further aspect, the present invention relates to a compound of formula (I) according to the first aspect of the invention or a pharmaceutical composition according to the second aspect of the invention when used in a method of treating or preventing a proliferative disease in a subject.

In a further aspect, the present invention relates to a composition having an active ingredient for use in a method of treating or preventing a proliferative disease in a subject, wherein the active ingredient is a compound of formula (I) according to the first aspect of the invention.

In a further aspect, the present invention relates to the use of a compound of formula (I) according to the first aspect of the invention or a pharmaceutical composition according to the second aspect of the invention in treating or preventing a proliferative disease in a subject, such as described herein.

In one embodiment, a compound of formula (I) according to the first aspect of the invention is the only active administered to the subject. In one embodiment, a compound of formula (I) according to the first aspect of the invention is the only active in the pharmaceutical composition.

In one or more preferred embodiments, the proliferative disease is cancer, preferably a solid tumour. In various preferred embodiments, the cancer is selected from the group consisting of breast cancer, lung cancer, prostate cancer, ovarian cancer, uterine cancer brain cancer, skin cancer, colon cancer and bladder cancer.

Those skilled in the art will understand that in the context of the present invention an ‘effective amount’ is an amount sufficient to produce a desired therapeutic or pharmacological effect in the subject being treated.

In a further aspect, the invention relates to a method of completely or partially preventing the recurrence of a solid tumor in a subject, the method comprising administering to the subject an effective amount of a compound of formula (I) according to the first aspect of the invention or a pharmaceutical composition according to the second aspect of the invention.

In another aspect, the invention relates to the use of a compound of formula (I) according to the first aspect of the invention or the pharmaceutical composition according to the second aspect of the invention in the manufacture of a medicament for completely or partially preventing the recurrence of a solid tumor.

In a further aspect, the present invention relates to the use of a compound of formula (I) according to the first aspect of the invention or a pharmaceutical composition according to the second aspect of the invention for completely or partially preventing the recurrence of a solid tumor in a subject.

In a further aspect, the present invention relates to a compound of formula (I) according to the first aspect of the invention or a pharmaceutical composition according to the second aspect of the invention for use in completely or partially preventing the recurrence of a solid tumor in a subject.

In a further aspect, the present invention relates to a pharmaceutical composition for use in completely or partially preventing the recurrence of a solid tumor in a subject, in any of the embodiments described in the specification.

In a further aspect, the present invention relates to a compound of formula (I) according to the first aspect of the invention or a pharmaceutical composition according to the second aspect of the invention when used in a method of completely or partially preventing the recurrence of a solid tumor in a subject.

In a further aspect, the present invention relates to a composition having an active ingredient for use in a method of completely or partially preventing the recurrence of a solid tumor, wherein the active ingredient is a compound of formula (I) according to the first aspect of the invention.

In a further aspect, the present invention relates to the use of a compound of formula (I) according to the first aspect of the invention or a pharmaceutical composition according to the second aspect of the invention in completely or partially preventing the recurrence of a solid tumor, such as described herein.

In one embodiment, a compound of formula (I) according to the first aspect of the invention is the only active administered to the subject. In one embodiment, a compound of formula (I) according to the first aspect of the invention is the only active in the pharmaceutical composition.

The compounds of formula (I) may be used in therapy alone or in combination with one or more other chemotherapeutic agents, for example, as part of a combination therapy.

In another aspect, the present invention relates to a process for preparing a compound of formula (I) comprising the steps of:

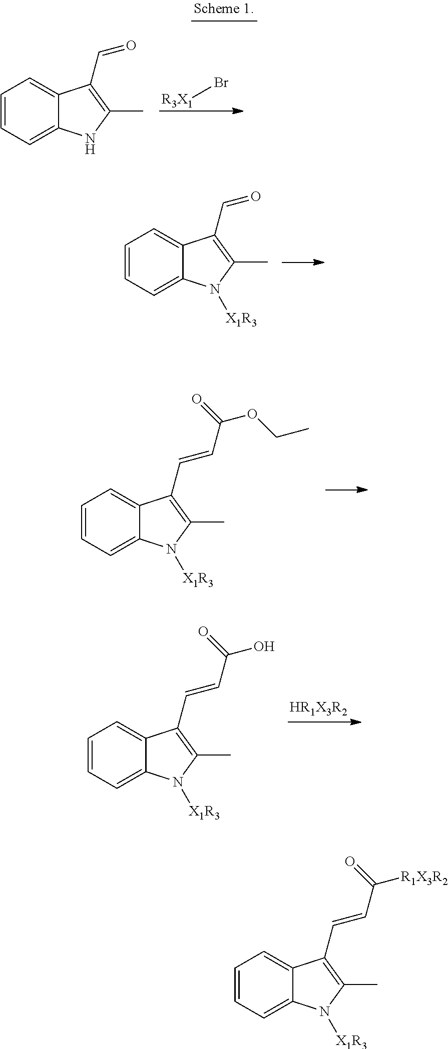

In another aspect the present invention relates to a process for preparing a compound of formula (I) comprising the steps of:

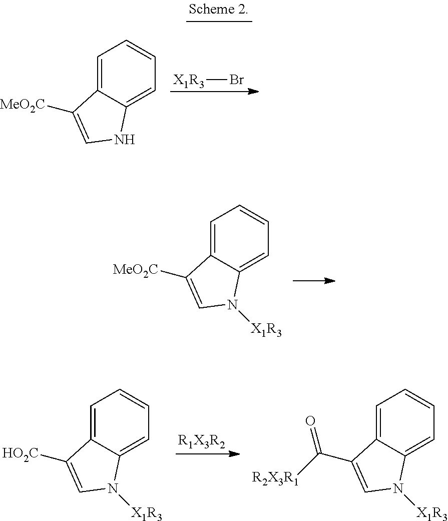

Further aspects of the present invention and further embodiments of the aspects described in the preceding paragraphs will become apparent from the following description, given by way of example and with reference to the accompanying drawings.

### DESCRIPTION OF THE DRAWINGS

FIG. 1: Impact of compound 2026 on Tm5NM1-regulated actin-filament depolymerization kinetics. (A and C) Depolymerization time course of 6 μM actin filaments (35% pyrene labelled) diluted 12-fold into F-actin buffer (100 mM NaCl, 10 mM Tris-HCl pH 7.0, 2 mM MgCl₂, 1 mM EGTA, 0.2 mM CaCl₂, 0.2 mM ATP, 0.5 mM DTT, 0.01% (v/v) NaN₃) in the presence or absence of saturating amounts (10 μM) of Tm5NM1. Final concentration of F-actin and Tm5NM1 was 0.5 μM and 0.83 μM respectively. Tm5NM1 was pre-incubated with 50 μM compound 2026 or 1% (v/v) DMSO prior to mixing with F-actin. Depolymerization data is normalized to the initial fluorescence value. (B and D) Initial rates (V₀) of depolymerization for F-actin alone or Tm5NM1/F-actin, in the presence of compound 2026. Initial rates of depolymerization were determined from the first 3600 s, fitted to a linear regression model. Data represents mean±SEM, averaged from n>6 replicates.

### DETAILED DESCRIPTION

The invention is based on the surprising finding that compounds of general formula (I) effectively inhibit tropomyosin, which results in unexpected improvement in the treatment of proliferative diseases, particularly cancer. The development of the actin cytoskeleton involves a number of ancillary control and regulatory proteins. Identification and specific targeting of actin regulatory proteins associated with the cytoskeleton of cancer cells offers the opportunity to develop cancer specific drugs without unwanted side effects.

Actin filaments are constructed through the polymersiation of globular actin protein monomers. The actin monomer is polar with one end bearing a positive charge and the other end a negative charge. The actin filaments thus have all the actin proteins aligned in one direction. These filaments have secondary coiled proteins tropomyosins associated with them. The tropomyosins play an integral role in regulating the function of actin filaments. Structurally the actin filaments are made up of polymeric actin monomers with tropomyosin dimers sitting in the alpha helical groove of the actin filament to form a homopolymer. There are more than 40 mammalian tropomyosin isoforms each of which regulates specific actin filaments. There are specific isoforms of tropomyosins that regulate the cytoskeleton of cancer cells, disruption of this interaction offers a basis to specifically treat cancer cells.

### I. DEFINITIONS

The following are some definitions of terms used in the art that may be helpful in understanding the description of the present invention. These are intended as general definitions and should in no way limit the scope of the present invention to those terms alone, but are put forth for a better understanding of the following description.

Unless the context requires otherwise or specifically states to the contrary, integers, steps, or elements of the invention recited herein as singular integers, steps or elements clearly encompass both singular and plural forms of the recited integers, steps or elements.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps, features, compositions and compounds. The terms “comprising” and “including” are used herein in their open-ended and non-limiting sense unless otherwise noted.

The term “optionally substituted” as used throughout the specification denotes that the group may or may not be further substituted or fused (so as to form a polycyclic system), with one or more non-hydrogen substituent groups. Suitable chemically viable optional substituents for a particular functional group will be apparent to those skilled in the art. Typical optional substituents include C₁-C₄ alkyl, C₂-C₄ alkenyl, OH, halogen, O(C₁-C₄ alkyl), NR^{a}R^{b} wherein R^{a} and R^{b} are independently selected from H, C₁-C₃ alkyl, CONH₂, SH, S(C₁-C₃ alkyl), —CH₂—O(C₁₋₃ alkyl), C₆₋₁₀ aryl, —CH₂-phenyl, hydroxyl-(C₁₋₃ alkyl), and halo-(C₁₋₃alkyl). Presently preferred optional substituents include C₁₋₃ alkyl, C₁₋₃ alkoxy, —CH₂—(C₁₋₃)alkoxy, C₆₋₁₀ aryl, —CH₂-phenyl, halogen, OH, hydroxy-(C₁₋₃)alkyl, and halo-(C₁₋₃)alkyl, e.g, CF₃, CH₂CF₃.

“Acyl” means an alkyl-CO— group in which the alkyl group is as described herein. Examples of acyl include acetyl and benzoyl. The alkyl group may be a C₁-C₆ alkyl, C₁-C₄ alkyl, or C₁-C₃ alkyl group. The group may be a terminal group or a bridging group.

“Alkyl” as a group or part of a group refers to a straight or branched aliphatic hydrocarbon group having 1-12 carbon atoms, or 1-10 carbon atoms, or 1-6 carbon atoms, or 1-4 carbon atoms, or 1-3 carbon atoms. Thus, for example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like. The group may be a terminal group or a bridging group.

“Alkenyl” as a group or part of a group denotes an aliphatic hydrocarbon group containing at least one carbon-carbon double bond and which may be straight or branched such as a group having 2-12 carbon atoms, or 2-6 carbon atoms, or 2-4 carbon atoms, in the normal chain. The group may contain a plurality of double bonds in the normal chain and the orientation about each double bond is independently cis or trans, E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like. The group may be a terminal group or a bridging group.

“Alkenyloxy” refers to an —O— alkenyl group in which alkenyl is as defined herein. Preferred alkenyloxy groups are C₂-C₁₂ alkenyloxy groups. The group may be a terminal group or a bridging group.

The terms “alkyloxy” and “alkoxy” are synonymous and refer to an —O-alkyl group in which alkyl is defined herein. Presently preferred alkoxy groups are C₁₋₆ alkoxy or C₁₋₄ alkoxy or C₁₋₃ alkoxy. Examples include, but are not limited to, methoxy, ethoxy, n-propoxy, isopropoxy, sec-butoxy, tert-butoxy, and the like. The group may be a terminal group or a bridging group.

“Alkylamino” includes both mono-alkylamino and dialkylamino, unless specified. “Mono-alkylamino” means a —NH-Alkyl group, in which alkyl is as defined above. “Dialkylamino” means a —N(alkyl)₂ group, in which each alkyl may be the same or different and are each as defined herein for alkyl. The alkyl group may be a C₁-C₆ alkyl group. The group may be a terminal group or a bridging group.

“Alkynyl” as a group or part of a group means an aliphatic hydrocarbon group containing a carbon-carbon triple bond and which may be straight or branched and may have from 2-12 carbon atoms or 2-6 carbon atoms or 2-4 carbon atoms in the normal chain. Exemplary structures include, but are not limited to, ethynyl and propynyl. The group may be a terminal group or a bridging group.

“Alkynyloxy” refers to an —O-alkynyl group in which alkynyl is as defined herein. Presently preferred alkynyloxy groups are C₂-C₆ alkynyloxy groups, C₂-C₄ alkynyloxy. The group may be a terminal group or a bridging group.

“Aryl” as a group or part of a group denotes (i) an optionally substituted monocyclic, or fused polycyclic, aromatic carbocycle (ring structure having ring atoms that are all carbon) that may have from 5-18 atoms per ring. Presently preferred aryl groups have 6-14 atoms per ring, or more preferably 6-10 atoms per ring. Examples of aryl groups include phenyl, naphthyl, phenanthryl and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group.

“Cycloalkenyl” means a non-aromatic monocyclic or multicyclic ring system containing at least one carbon-carbon double bond and may have from 5-10 carbon atoms per ring. Exemplary monocyclic cycloalkenyl rings include cyclopentenyl, cyclohexenyl or cycloheptenyl. The cycloalkenyl group may be substituted by one or more substituent groups. The group may be a terminal group or a bridging group.

“Cycloalkyl” refers to a saturated or partially saturated, monocyclic or fused or spiro polycyclic, carbocycle that may contain from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

The terms “halogen” or “halo” are synonymous and refer to fluorine, chlorine, bromine or iodine.

“Heteroaryl” either alone or as part of a group refers to groups containing an aromatic ring (such as a 5- or 6-membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

The term “heteroatom” or variants such as “hetero-” as used herein refers to O, N, NH and S.

Certain compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, formula (I) is intended to cover, where applicable, solvated as well as unsolvated forms of the compounds. Thus, formula (I) includes compounds having the indicated structure, including the hydrated or solvated form, as well as the non-hydrated and non-solvated forms.

The term “pharmaceutically acceptable salt” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, salicylic, p-hydroxybenzoic, phenylacetic, mandelic, ambonic, pamoic, pantothenic, sulfanilic, cyclohexylaminosulfonic, stearic, algenic, β-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, organic salts made from N,N′-dibenzylethylenediamine, chloroprocaine, choline, diethanolamine, ethylenediamine, meglumine (N-methylglucamine), procaine, ammonium salts, quaternary salts such as tetramethylammonium salt, amino acid addition salts such as salts with glycine and arginine. In the case of compounds that are solids, it will be understood by those skilled in the art that the inventive compounds, agents and salts may exist in different crystalline or polymorphic forms, all of which are intended to be within the scope of the present invention and specified formulae.

“Prodrug” means a compound which is convertible in vivo by metabolic means (e.g. by hydrolysis, reduction or oxidation) to a compound of the present invention. For example an ester prodrug of a compound of the present invention containing a hydroxyl group may be convertible by hydrolysis in vivo to the parent molecule. Suitable esters are for example, acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of cancer or its associated symptoms.

“Preventing” or “prevention” means preventing the occurrence of the cancer or tempering the severity of the cancer if it develops subsequent to the administration of the compounds or pharmaceutical compositions of the present invention. This prevents the onset of clinically evident unwanted cell proliferation altogether or the onset of a preclinically evident stage of unwanted rapid cell proliferation in individuals at risk. Also intended to be encompassed by this definition is the prevention of metastases of malignant cells or the arrest or reversal of the progression of malignant cells.

The terms “therapeutically effective” or “pharmacologically effective” are intended to qualify the amount of each agent which will achieve the goal of improvement in disease severity and the frequency of incidence over treatment of each agent by itself while avoiding adverse side effects typically associated with other therapies.

A “pharmaceutical carrier, diluent or excipient” includes, but is not limited to, any physiological buffered (i.e., about pH 7.0 to 7.4) medium comprising a suitable water soluble organic carrier, conventional solvents, dispersion media, fillers, solid carriers, coatings, antibacterial and antifungal agents, isotonic and absorption delaying agents. Suitable water soluble organic carriers include, but are not limited to saline, dextrose, corn oil, dimethylsulfoxide, and gelatin capsules. Other conventional additives include lactose, mannitol, corn starch, potato starch, binders such as crystalline cellulose, cellulose derivatives, acacia, gelatins, disintegrators such as sodium carboxymethyl-cellulose, and lubricants such as talc or magnesium stearate.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

In the context of this specification the term “administering” and variations of that term including “administer” and “administration”, includes contacting, applying, delivering or providing a compound or composition of the invention to an organism, or a surface by any appropriate means.

### II. SYNTHESIS OF COMPOUNDS OF THE INVENTION

The present invention relates to functionalized indole compounds of general formula (I) as defined herein, and to the use of such compounds as anticancer agents.

Compounds of general formula (I), or salts, hydrates or solvates thereof, may be prepared by methods known to those skilled in the art. The general synthetic schemes for preparing compounds of formula (I) are described below:

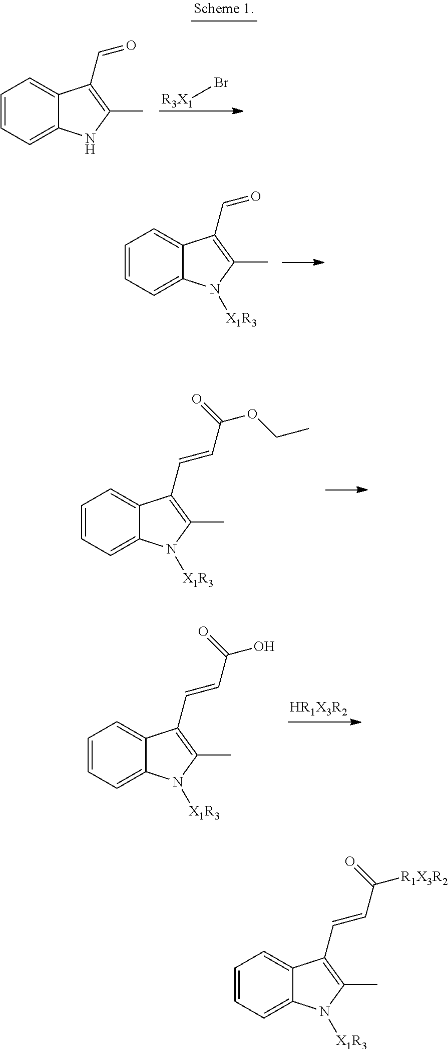

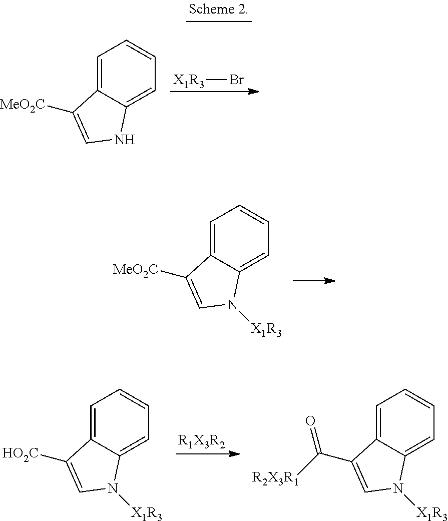

The methods described above in Schemes 1-2 may offer one or more advantages including high yields, control of stereochemistry, few synthetic steps and reaction conditions that are amenable to large scale manufacture.

The methods described above are merely representative and routine modifications and variations that would be apparent to persons skilled in the art fall within the broad scope and ambit of the invention disclosed herein.

### III. METHODS OF TREATMENT USING COMPOUNDS OF THE INVENTION

The compounds of general formula (I) according to the present invention, and pharmaceutical compositions thereof, may be used in the treatment or prevention of proliferative diseases, preferably cancer. The compounds and compositions of the invention may be useful for the treatment of a wide variety of cancers (tumours), including but not limited to, solid tumours, such as for example, breast cancer, lung cancer, prostate cancer, ovarian cancer, uterine cancer brain cancer, skin cancer, colon cancer and bladder cancer.

Advantageously, compounds of the present invention may possess superior pharmaceutical properties, such as improved resistance to conjugation via glucuronyl transferases and other water solubilizing transferases such as sulfases, which may be over-expressed on proliferative cells such as cancer cells. This may advantageously confer superior pharmaceutical properties, such as an enhanced pharmacokinetic profile through reduced conjugation and elimination.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds or pharmaceutical compositions of the present invention may be administered orally, intravenously, intranasally, rectally, parenterally, subcutaneously, intramuscularly, topically or by any means which delivers an effective amount of the active agent to the tissue or site to be treated. It will be appreciated that different dosages may be required for treating different disorders. An effective amount of an agent is that amount which causes a statistically significant decrease in neoplastic cell count, growth, or size. Neoplastic disorders responsive to the agents of the present invention include, but are not limited to, breast cancer.

The dosage form and amount of the compounds or pharmaceutical compositions of the present invention can be readily established by reference to known treatment or prophylactic regimens.

For example, the compounds and pharmaceutical compositions may be formulated for oral, injectable, rectal, parenteral, subcutaneous, intravenous or intramuscular delivery. Non-limiting examples of particular formulation types include tablets, capsules, caplets, powders, granules injectables, ampoules, vials, ready-to-use solutions or suspensions, lyophilized materials, suppositories and implants. The solid formulations such as the tablets or capsules may contain any number of suitable pharmaceutically acceptable excipients or carriers described above.

For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials.

The amount of therapeutically effective compound that is administered and the dosage regimen for treating a disease condition with the compounds and/or pharmaceutical compositions of the invention depends on a variety of factors, including the age, weight, sex, and medical condition of the subject, the severity of the disease, the route and frequency of administration, the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

The compounds of the present invention may be administered along with a pharmaceutical carrier, diluent or excipient as described above. Alternatively, or in addition to, the compounds may be administered in combination with other agents, for example, chemotherapeutic or immune-stimulating drugs or therapeutic agents.

The terms “combination therapy” or “adjunct therapy” in defining use of a compound of the present invention and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments of the present invention one or more compounds of general formula (I) may be formulated or administered in combination with one or more other therapeutic agents. Thus, in accordance with various embodiments of the present invention, one or more compounds of general formula (I) may be included in combination treatment regimens with surgery and/or other known treatments or therapeutic agents, such as other anticancer agents, in particular, chemotherapeutic agents, radiotherapeutic agents, and/or adjuvant or prophylactic agents.

There are large numbers of antineoplastic agents available in commercial use, in clinical evaluation and in pre-clinical development, which could be selected for treatment of cancers or other neoplasias by combination drug chemotherapy. Such anti-neoplastic agents fall into several major categories, namely, antibiotic-type agents, antimetabolite agents, hormonal agents, immunological agents, interferon-type agents and a category of miscellaneous agents. Alternatively, other anti-neoplastic agents, such as metallomatrix proteases inhibitors may be used. Suitable agents which may be used in combination therapy will be recognized by those of skill in the art. Suitable agents are listed, for example, in the Merck Index, An Encyclopaedia of Chemicals, Drugs and Biologicals, 12^{th} Ed., 1996, the entire contents of which are incorporated herein by reference.

Combination regimens may involve the active agents being administered together, sequentially, or spaced apart as appropriate in each case. Combinations of active agents including compounds of the invention may be synergistic.

The co-administration of compounds of the general formula (I) may be effected by a compound of the general formula (I) being in the same unit dose as a chemotherapeutic or other anti-cancer agent, or the compound of the general formula (I) and the chemotherapeutic or other anti-cancer agents may be present in individual and discrete unit doses administered at the same, or at a similar time. Sequential administration may be in any order as required, and may require an ongoing physiological effect of the first or initial compound to be current when the second or later compound is administered, especially where a cumulative or synergistic effect is desired.

Embodiments of the invention will now be discussed in more detail with reference to the examples which is provided for exemplification only and which should not be considered limiting on the scope of the invention in any way.

### Examples

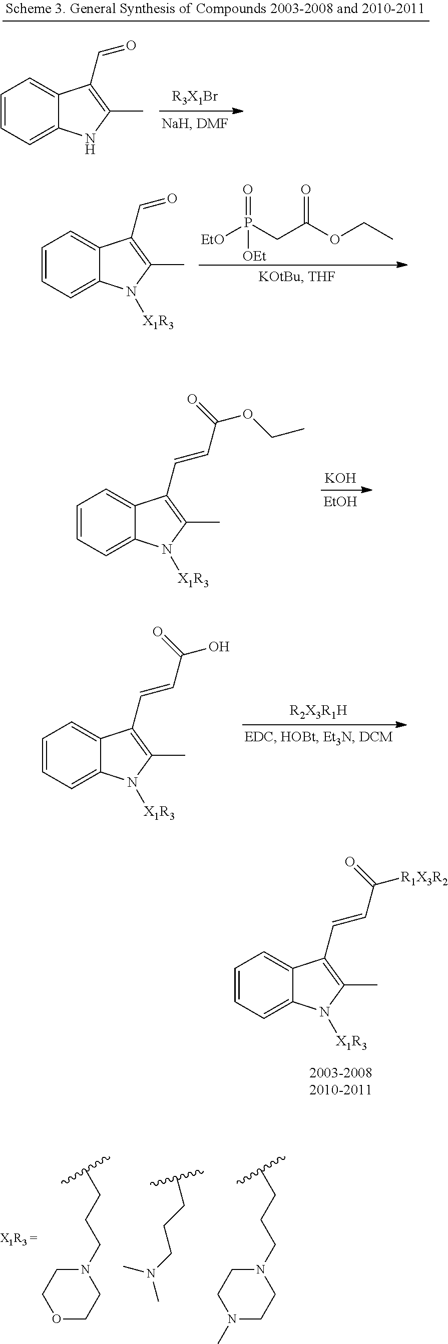

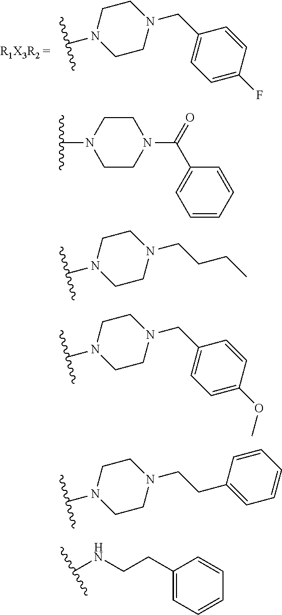

### Preparation of 1-(3-(dimethylamino)propyl)-2-methyl-1H-indole-3-carbaldehyde

To the stirred mixture of 2-methyl-1H-indole-3-carbaldehyde (5.0 g, 31.4 mmol) in DMF, NaH (5.7 g, 47.1 mmol) was added portionwise under ice-water bath conditions. The mixture was then stirred at 0° C. for 1 hour. 3-Chloro-N,N-dimethylpropan-1-amine hydrochloride (5.7 g, 47.1 mmol) was added, then the mixture was warmed up to 75° C. for 3 hours. Water was added, the aqueous was extracted with ethyl acetate, and the organic phase was washed with water and brine, dried over Na₂SO₄, concentrated in vacuum and purified by gel column to give a yellow oil, (4.2 g, 55%).

### Preparation of (E)-ethyl 3-(1-(3-(dimethylamino)propyl)-2-methyl-1H-indol-3-yl)acrylate

To the stirred solution of ethyl 2-(diethoxyphosphoryl)acetate (3.5 g, 15.8 mmol) in THF, potassium tert-butoxide (2.2 g, 20.0 mmol) was added portionwise under ice-water bath, and then the mixture was stirred at room temperature for 2 hours. 1-(3-(Dimethylamino)propyl)-2-methyl-1H-indole-3-carbaldehyde (3.5 g, 14.3 mmol) was added under ice-water bath, then the mixture was warmed up to room temperature overnight. Water was added, the aqueous was extracted with ethyl acetate, the organic phase was washed with brine, dried over Na₂SO₄, concentrated in vacuum and purified by chromatography (DCM/MeOH=100:1) to give a brown oil (1.8 g, 40%).

¹H NMR (400 MHz, CDCl₃): δ 7.98 (d, J=15.6 Hz, 1H), 7.88 (m, 1H), 7.38 (m, 1H), 7.23-7.21 (m, 2H), 6.42 (d, J=16.4 Hz, 1H), 4.28-4.27 (m, 2H), 4.19 (t, J=7.2 Hz, 2H), 2.57 (s, 3H), 2.74-2.72 (m, 2H), 2.32 (t, J=7.2 Hz, 2H), 2.31 (s, 6H), 1.95-1.93 (m, 2H), 1.36 (t, J=6.8 Hz, 3H).

### Preparation of (E)-3-(1-(3-(dimethylamino)propyl)-2-methyl-1H-indol-3-yl)-acrylic acid

To a stirred solution of (E)-ethyl 3-(1-(3-(dimethylamino)propyl)-2-methyl-1H-indol-3-yl)acrylate (1.8 g, 5.7 mmol) in EtOH, a solution of KOH (1.6 g, 28.6 mmol) in water was added under ice-water bath, and then the mixture was stirred at 40° C. overnight. The mixture was concentrated in vacuum at 40° C. to afford a yellow solid (containing some salts) (3.7 g, quantitative yield). The solid was used in the next step without any purification.

### Preparation of Compound 2003, (E)-3-(1-(3-(dimethylamino)propyl)-2-methyl-1H-indol-3-yl)-1-(4-(4-fluorobenzyl)piperazin-1-yl)prop-2-en-1-one

To a stirred suspension of (E)-3-(1-(3-(dimethylamino)propyl)-2-methyl-1H-indol-3-yl) acrylic acid (100 mg, 0.35 mmol) in DCM was added 1-(4-fluorobenzyl)piperazine (101 mg, 0.52 mmol). EDC (134 mg, 0.7 mmol) and HOBt (94.5 mg, 0.7 mmol) were then added one by one at 0° C. The mixture was then allowed to warm to room temperature and stirred overnight. The resultant was quenched with water, extracted with ethyl acetate, washed with solutions of NH₄Cl and brine, concentrated and purified by prep-HPLC to give a colorless oil (25 mg, 16%).

¹H NMR (400 MHz, CDCl₃): δ 8.01 (d, J=14.8 Hz, 1H), 7.81 (dd, J=6.8 Hz, 2.0 Hz, 1H), 7.38 (dd, J=7.2 Hz, 2.0 Hz, 1H), 7.31 (dd, J=8.4 Hz, 5.6 Hz, 2H), 7.24-7.18 (m, 2H), 6.85 (d, J=15.2 Hz, 1H), 4.18 (t, J=7.2 Hz, 2H), 3.72 (m, 4H), 3.51 (s, 2H), 2.56 (s, 3H), 2.49 (t, J=4.8 Hz, 4H), 2.27 (t, J=6.8 Hz, 2H), 2.23 (s, 6H), 1.94-1.89 (m, 2H).

### Preparation of Compound 2004, (E)-1-(4-benzoylpiperazin-1-yl)-3-(1-(3-(dimethylamino)propyl)-2-methyl-1H-indol-3-yl)prop-2-en-1-one

To a stirred suspension of (E)-3-(1-(3-(dimethylamino)propyl)-2-methyl-1H-indol-3-yl)acrylic acid (86 mg, 0.3 mmol) in DCM was added phenyl (piperazin-1-yl) methanone (86 mg, 0.45 mmol). Et₃N (60 mg, 0.6 mmol), EDC (115 mg, 0.6 mmol) and HOBt (81 mg, 0.6 mmol) were then added one by one at 0° C. The mixture was then allowed to warm to room temperature and stirred overnight. The resultant was quenched with water, extracted with ethyl acetate, washed with solutions of NH₄Cl and brine, concentrated and purified by prep-HPLC to give a colorless oil (29 mg, 20%).

¹H NMR (400 MHz, CDCl₃): δ 8.07 (d, J=15.6 Hz, 1H), 7.83 (s, 1H), 7.46-7.39 (m, 6H), 7.29-7.24 (m, 2H), 6.85 (d, J=16.4 Hz, 1H), 4.21 (t, J=7.2 Hz, 2H), 3.86-3.74 (m, 6H), 3.56-3.52 (m, 2H), 2.59 (s, 3H), 2.31 (t, J=6.4 Hz, 2H), 2.26 (s, 6H), 1.97-1.91 (m, 2H).

### Preparation of Compound 2005, (E)-1-(4-butylpiperazin-1-yl)-3-(1-(3-(dimethylamino)propyl)-2-methyl-1H-indol-3-yl)prop-2-en-1-one

To a stirred suspension of (E)-3-(1-(3-(dimethylamino)propyl)-2-methyl-1H-indol-3-yl)acrylic acid (86 mg, 0.3 mmol) in DCM was added 1-butylpiperazine (64 mg, 0.45 mmol). Et₃N (60 mg, 0.6 mmol), EDC (115 mg, 0.6 mmol) and HOBt (81 mg, 0.6 mmol) were then added one by one at 0° C. The mixture was then allowed to warm to room temperature and stirred overnight. The resultant was quenched with water, extracted with ethyl acetate, washed with solutions of NH₄Cl and brine, concentrated and purified by prep-HPLC to give a colorless oil (43 mg, 34%).

¹H NMR (400 MHz, CDCl₃): δ 8.02 (d, J=15.6 Hz, 1H), 7.84 (dd, J=7.6 Hz, 1.6 Hz, 1H), 7.39 (dd, J=6.4 Hz, 2.4 Hz, 1H), 7.25-7.22 (m, 2H), 6.88 (d, J=14.8 Hz, 1H), 4.19 (t, J=7.2 Hz, 2H), 3.78 (br s, 4H), 2.57 (s, 3H), 2.51 (br s, 4H), 2.39 (t, J=7.6 Hz, 2H), 2.28 (t, J=6.4 Hz, 2H), 2.24 (s, 6H), 1.95-1.88 (m, 2H), 1.54-1.50 (m, 2H), 1.39-1.34 (m, 2H), 0.95 (t, J=7.2 Hz, 3H).

### Preparation of Compound 2006, (E)-3-(1-(2-(dimethylamino)ethyl)-2-methyl-1H-indol-3-yl)-1-(4-phenethylpiperazin-1-yl)prop-2-en-1-one

To a stirred solution of 1-phenethylpiperazine hydrochloride (61.02 mg, 0.27 mmol) and Et₃N (37 mg, 0.36 mmol) in DCM was added (E)-3-(1-(2-(dimethylamino)ethyl)-2-methyl-1H-indol-3-yl)acrylic acid (50 mg, 0.18 mmol). The mixture was stirred at room temperature for 15 min. Then, EDC (52 mg, 0.27 mmol) and HOBt (37 mg, 0.27 mmol) were added. Thereafter, the mixture was stirred at room temperature overnight. The mixture was poured into water, and extracted with DCM. The organic layer was washed with water and brine. After being concentrated in vacuum, it was purified by prep-HPLC to give a colorless oil (15 mg, 18%).

¹H NMR (400 MHz, CDCl₃): δ 8.00 (d, J=13.6 Hz, 1H), 7.82 (br d, J=8.0 Hz, 1H), 7.35-7.21 (m, 8H), 6.86 (d, J=16.0 Hz, 1H), 4.24 (t, J=7.2 Hz, 2H), 3.80 (br s, 4H), 2.88-2.83 (m, 2H), 2.69-2.60 (m, 8H), 2.55 (s, 3H), 2.36 (s, 6H).

### Preparation of Compound 2007, (E)-3-(2-methyl-1-(3-morpholinopropyl)-1H-indol-3-yl)-1-(4-phenethylpiperazin-1-yl)prop-2-en-1-one

To the suspension of (E)-3-(2-methyl-1-(3-morpholinopropyl)-1H-indol-3-yl)acrylic acid (85 mg, 0.26 mmol), EDC (99 mg, 0.52 mmol), and HOBt (70 mg, 0.52 mmol) in DCM (5 mL) at 0° C. was added 1-phenethylpiperazine hydrochloride (176 mg, 0.78 mmol) and Et₃N (79 mg, 0.78 mmol) in DCM. The mixture was warmed up to room temperature and stirred for 4 hours. TLC showed the starting material had been consumed. Then the mixture was poured into water, extracted with DCM, washed with NH₄Cl(aq) and NaHCO₃(aq) and dried over Na₂SO₄. The DCM layer was concentrated in vacuum and purified by prep-HPLC to afford a colorless oil (20 mg, 16%).

¹H NMR (400 MHz, CDCl₃): δ 8.03 (d, J=12.4 Hz, 1H), 7.85 (br d, J=5.2 Hz, 1H), 7.40 (br d, J=3.6 Hz, 1H), 7.35-7.20 (m, 8H), 6.88 (d, J=16.8 Hz, 1H), 4.22 (t, J=6.8 Hz, 2H), 3.79-3.74 (m, 8H), 2.86 (t, J=7.6 Hz, 2H), 2.68-2.58 (m, 9H), 2.41 (br s, 4H), 2.32 (t, J=7.2 Hz, 2H), 1.95 (quintet, J=6.4 Hz, 2H).

### Preparation of Compound 2008, (E)-3-(2-methyl-1-(3-(4-methylpiperazin-1-yl)propyl)-1H-indol-3-yl)-1-(4-phenethylpiperazin-1-yl)prop-2-en-1-one

To the suspension of (E)-3-(2-methyl-1-(3-(4-methylpiperazin-1-yl)propyl)-1H-indol-3-yl) acrylic acid (100 mg, 0.3 mmol), EDC (81 mg, 0.6 mmol), and HOBt (115 mg, 0.6 mmol) in DCM (5 mL) at 0° C. was added 1-phenethylpiperazine hydrochloride (101 mg, 0.45 mmol) and Et₃N (61 mg, 0.6 mmol) in DCM. The mixture was warmed up to room temperature and stirred for 4 hours. TLC showed the starting material had been consumed. Then the mixture was poured into water, extracted with DCM, washed by NH₄Cl(aq) and NaHCO₃(aq) and dried over Na₂SO₄. The DCM layer was concentrated in vacuum and purified by prep-HPLC to afford a colorless oil (30 mg, 20%).

¹H NMR (400 MHz, CDCl₃): δ 8.00 (d, J=15.2 Hz, 1H), 7.81 (br d, J=9.2 Hz, 1H), 7.38 (dd, J=8.0 Hz, 2.0 Hz, 1H), 7.31-7.26 (m, 2H), 7.25-7.19 (m, 5H), 6.85 (d, J=14.8 Hz, 1H), 4.17 (t, J=7.2 Hz, 2H), 3.78 (m, 4H), 2.85-2.81 (m, 2H), 2.66-2.33 (m, 17H), 2.32-2.28 (m, 5H), 1.92 (quintet, J=7.2 Hz, 2H).

### Preparation of Compound 2010, (E)-3-(1-(3-(dimethylamino)propyl)-2-methyl-1H-indol-3-yl)-1-(4-(4-methoxybenzyl) piperazin-1-yl)prop-2-en-1-one

To a stirred suspension of (E)-3-(1-(3-(dimethylamino)propyl)-2-methyl-1H-indol-3-yl)acrylic acid (86 mg, 0.3 mmol) in DCM was added 1-(4-methoxybenzyl) piperazine (92 mg, 0.45 mmol), Et₃N (60 mg, 0.6 mmol), EDC (115 mg, 0.6 mmol) and HOBt (81 mg, 0.6 mmol) was then added one by one at 0° C. The mixture was then allowed to warm to room temperature and stirred overnight. The resultant was quenched with water, extracted with ethyl acetate, washed with solutions of NH₄Cl and brine, concentrated and purified by prep-HPLC to give a colorless oil (67 mg, 47%).

¹H NMR (400 MHz, CDCl₃): δ 8.02 (d, J=14.8 Hz, 1H), 7.82 (dd, J=7.2 Hz, 2.0 Hz, 1H), 7.39 (dd, J=7.2 Hz, 1.2 Hz, 1H), 7.28-7.21 (m, 4H), 6.90-6.85 (m, 3H), 4.19 (t, J=7.2 Hz, 2H), 3.83 (s, 3H), 3.78 (br s, 4H), 3.50 (s, 2H), 2.57 (s, 3H), 2.50 (t, J=4.4 Hz, 4H), 2.28 (t, J=6.4 Hz, 2H), 2.24 (s, 6H), 1.91 (quintet, J=6.8 Hz, 2H).

### Preparation of Compound 2011 (E)-3-(1-(3-(dimethylamino)propyl)-2-methyl-1H-indol-3-yl)-N-phenethylacrylamide

To a stirred solution of (E)-3-(1-(3-(dimethylamino)propyl)-2-methyl-1H-indol-3-yl) acrylic acid (110 mg, 0.38 mmol), EDC (145 mg, 0.76 mmol) and HOBt (102 mg, 0.76 mmol) in DCM, a solution of 2-phenylethanamine (131 mg, 1.14 mmol) in DCM was added under ice-water bath, and then the mixture was stirred at room temperature for 1.5 hours. The mixture solution was washed with saturated NH₄Cl aqueous solution, saturated NaHCO₃ aqueous solution, dried over Na₂SO₄, concentrated in vacuum and crystallized with petroleum ether/ethyl acetate to give a yellow solid (21 mg, 14%).

¹H NMR (400 MHz, CDCl₃): δ 7.94 (d, J=14.4 Hz, 1H), 7.80 (br d, J=8.0 Hz, 1H), 7.38-7.32 (m, 3H), 7.25-7.20 (m, 5H), 6.32 (d, J=15.2 Hz, 1H), 5.55 (br s, 1H), 4.18 (t, J=7.2 Hz, 2H), 3.69 (q, J=6.0 Hz, 2H), 2.92 (t, J=6.0 Hz, 2H), 2.58 (s, 3H), 2.29 (t, J=6.8 Hz, 2H), 2.25 (s, 6H), 1.94-1.92 (m, 2H).

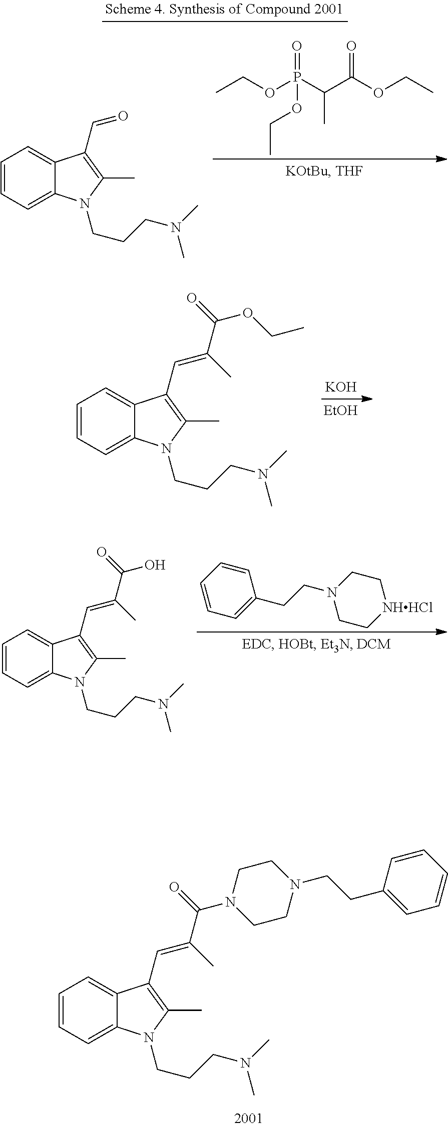

### Preparation of (E)-ethyl 3-(1-(3-(dimethylamino)propyl)-2-methyl-1H-indol-3-yl)but-2-enoate

To the solution of ethyl 2-(diethoxyphosphoryl)propanoate (535 mg, 2.25 mmol) in THF, potassium tert-butoxide was added at 0° C. Then the solution was stirred at room temperature for 2 hours. The solution was cooled down again to 0° C., and a solution of 1-(3-(dimethylamino)propyl)-2-methyl-1H-indole-3-carbaldehyde (500 mg, 2.25 mmol) in THF was added dropwise. The solution was stirred at room temperature overnight. The mixture was poured into water and extracted with ethyl acetate, washed with water and brine and dried over Na₂SO₄. The resultant was concentrated in vacuum and purified by passing through a short silica gel column (CH₂Cl₂: MeOH=50:1) to give a colorless oil (200 mg, 30%).

¹H NMR (400 MHz, DMSO-d₆): δ 7.76 (s, 1H), 7.47 (d, J=7.6 Hz, 1H), 7.36 (d, J=7.6 Hz, 1H), 7.14 (t, J=7.6 Hz, 1H), 7.06 (t, J=7.6 Hz, 1H), 4.21-4.18 (m, 4H), 2.38 (s, 3H), 2.20 (t, J=6.4 Hz, 2H), 2.14 (s, 6H), 1.89 (s, 3H), 1.83-1.81 (m, 2H), 1.29 (t, J=7.2 Hz, 3H).

### Preparation of (E)-3-(1-(3-(dimethylamino)propyl)-2-methyl-1H-indol-3-yl)but-2-enoic acid

To the solution of (E)-ethyl 3-(1-(3-(dimethylamino)propyl)-2-methyl-1H-indol-3-yl)but-2-enoate (200 mg, 0.61 mmol) in EtOH (5 mL) was added a solution of KOH (170 mg, 3.05 mmol) in H₂O (5 mL). The reaction mixture was heated at 50° C. for 6 hours. TLC showed that the starting material had disappeared. The mixture was concentrated in vacuum, then adjusted to pH 2. Then the mixture was extracted with ethyl acetate. The organic layer was washed with water and brine, dried over NaSO₄ and concentrated in vacuum to give a white solid (150 mg, 80%).

### Preparation of Compound 2001, (E)-3-(1-(3-(dimethylamino)propyl)-2-methyl-1H-indol-3-yl)-1-(4-phenethylpiperazin-1-yl)but-2-en-1-one

To a solution of (E)-3-(1-(3-(dimethylamino)propyl)-2-methyl-1H-indol-3-yl)but-2-enoic acid (100 mg, 0.334 mmol), EDC (128 mg, 0.668 mmol), HOBt (90 mg, 0.668 mmol) at 0° C. in DCM (5 mL), 1-phenethylpiperazine hydrochloride (226 mg, 1.00 mmol) and Et₃N (101 mg, 101 mmol) in DCM was added. The mixture was warmed up to room temperature and stirred for 4 hours. TLC showed the starting material had been consumed. Then the mixture was poured into water, extracted with ethyl acetate, washed with water and brine and dried over Na₂SO₄. The mixture was concentrated in vacuum and purified by prep-TLC to afford a colorless oil (50 mg, 30%).

¹H NMR (400 MHz, CDCl₃): δ 7.41 (d, J=7.2 Hz, 1H), 7.33-7.12 (m, 8H), 6.62 (br s, 1H), 4.21 (t, J=7.2 Hz, 2H), 3.78 (br t, J=4.8 Hz, 4H), 2.86-2.82 (m, 2H), 2.68-2.64 (m, 2H), 2.59 (br t, J=4.8 Hz, 4H), 2.50 (t, J=6.8 Hz, 2H), 2.39 (s, 3H), 2.37 (s, 6H), 2.09-2.05 (m, 2H), 1.93 (s, 3H).

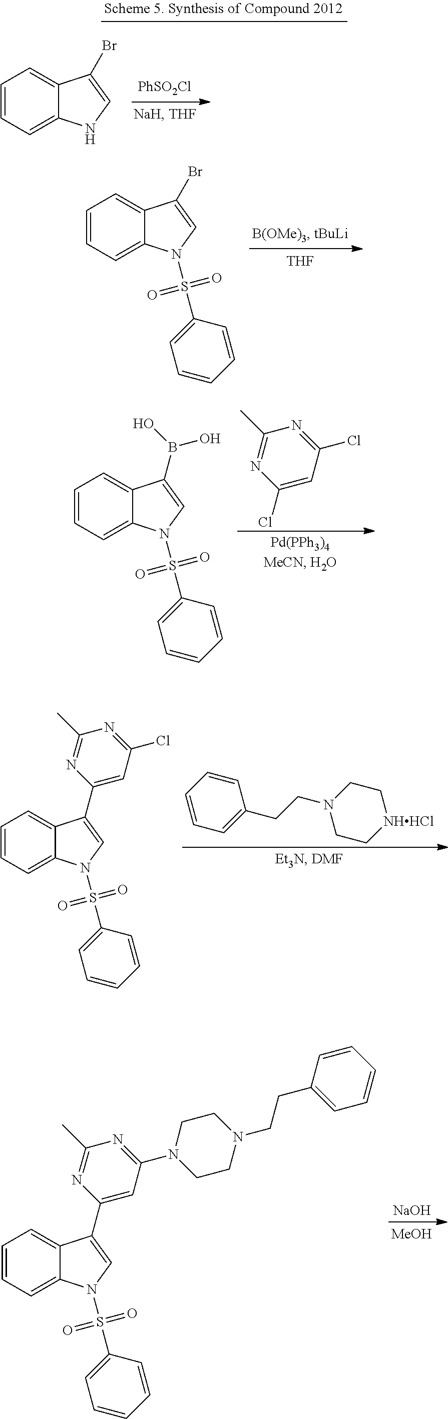

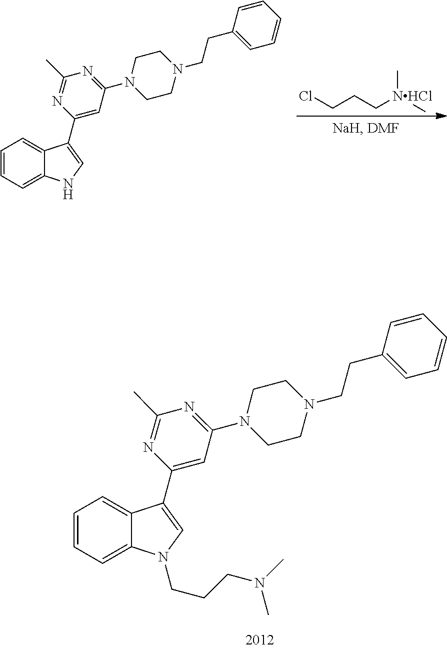

### Preparation of 3-bromo-1-(phenylsulfonyl)-1H-indole

To a solution of 3-bromo-1H-indole (600 mg, 3.06 mmol) in THF (40 mL) was added NaH (245 mg, 6.12 mmol) over an ice-water bath. Benzenesulfonyl chloride (541 mg, 3.06 mmol) was added into the mixture dropwise. The resultant was stirred at room temperature for 1.5 hours. The reaction was quenched by slowly adding water. The mixture was then poured into water, extracted with ethyl acetate, washed with water and brine and dried over anhydrous NaSO₄. The resultant was concentrated to afford a white solid (1 g, 97%).

### Preparation of 1-(phenylsulfonyl)-1H-indol-3-ylboronic acid

To a stirred suspension of 3-bromo-1-(phenylsulfonyl)-1H-indole (50 mg, 0.15 mmol) in THF was added t-BuLi (1.6 M, 0.19 mL) at −78° C., stirred for 5 minutes. Trimethyl borate (47 mg, 0.45 mmol) was added in one portion at the same temperature and stirring was continued for 2 hours. The resultant was quenched by aqueous HCl and extracted with DCM. The DCM layer was dried by Na₂SO₄ and concentrated. The concentrate was used in the next step without any purification.

### Preparation of 3-(6-chloro-2-methylpyrimidin-4-yl)-1-(phenylsulfonyl)-1H-indole

To a stirred suspension of 1-(phenylsulfonyl)-1H-indol-3-ylboronic acid (50 mg, 0.17 mmol) in MeCN/H₂O was added 4,6-dichloro-2-methylpyrimidine (74.7 mg, 0.30 mmol), Pd(PPh₃)₄ and aqueous Na₂CO₃. The reaction mixture was heated at reflux overnight. The resultant was quenched with water, extracted with ethyl acetate and then purified by silica gel plates to give a white solid (20 mg, 23%).

¹H NMR (400 MHz, DMSO-d₆): δ 8.07 (d, J=8.4 Hz, 1H), 7.93 (s, 1H), 7.84 (d, J=8.0 Hz, 2H), 7.70-7.65 (m, 2H), 7.57 (t, J=8.0 Hz, 2H), 7.48 (t, J=8.0 Hz, 1H), 7.34 (t, J=7.6 Hz, 2H), 2.64 (s, 3H).

### Preparation of 3-(2-methyl-6-(4-phenethylpiperazin-1-yl)pyrimidin-4-yl)-1-(phenylsulfonyl)-1H-indole

To a stirred suspension of 3-(6-chloro-2-methylpyrimidin-4-yl)-1-(phenylsulfonyl)-1H-indole (40 mg, 0.11 mmol) in DMF was added 1-phenethylpiperazine hydrochloride (29 mg, 0.13 mmol) and Et₃N (33 mg, 0.33 mmol). The reaction mixture was heated to 80° C. overnight. The resultant was quenched with water and extracted with ethyl acetate. The ethyl acetate layer was washed with aqueous NH₄Cl and brine, and then purified by silica gel plates to give a yellow oil (30 mg, 54%).

### Preparation of 3-(2-methyl-6-(4-phenethylpiperazin-1-yl)pyrimidin-4-yl)-1H-indole

To a stirred suspension of 3-(2-methyl-6-(4-phenethylpiperazin-1-yl)pyrimidin-4-yl)-1-(phenylsulfonyl)-1H-indole (76 mg, 0.13 mmol) in MeOH was added NaOH (52 mg, 1.3 mmol). The reaction mixture was heated at reflux overnight. The resultant was concentrated directly; water and ethyl acetate were then added. The organic layer was concentrated to give a yellow oil (41 mg, 80%), which was used in the next step directly.

### Preparation of Compound 2012, N,N-dimethyl-3-(3-(2-methyl-6-(4-phenethylpiperazin-1-yl)pyrimidin-4-yl)-1H-indol-1-yl)propan-1-amine

To a stirred suspension of 3-(2-methyl-6-(4-phenethylpiperazin-1-yl)pyrimidin-4-yl)-1H-indole (47 mg, 0.12 mmol) in DMF was added NaH at 0° C. The mixture was stirred at the same temperature for 30 minutes before 3-chloro-N,N-dimethylpropan-1-amine hydrochloride (20.5 mg, 0.13 mmol) was added. The reaction mixture was heated to 80° C. for 2 hours. The resultant was quenched with water and extracted with ethyl acetate, concentrated and purified by silica gel plates to give a colorless oil (35 mg, 61%).

¹H NMR (400 MHz, CDCl₃): δ 7.62 (d, J=7.6 Hz, 1H), 7.44 (d, J=7.6 Hz, 1H), 7.33-7.20 (m, 6H), 7.11 (t, J=8.0 Hz, 1H), 6.87 (d, J=2.8 Hz, 1H), 6.70 (d, J=2.0 Hz, 1H), 4.66 (t, J=7.2 Hz, 1H), 3.75 (m, 4H), 2.89-2.84 (m, 2H), 2.70-2.58 (m, 6H), 2.57 (s, 3H), 2.29-2.25 (m, 2H), 2.19 (s, 6H), 1.97-1.94 (m, 2H).

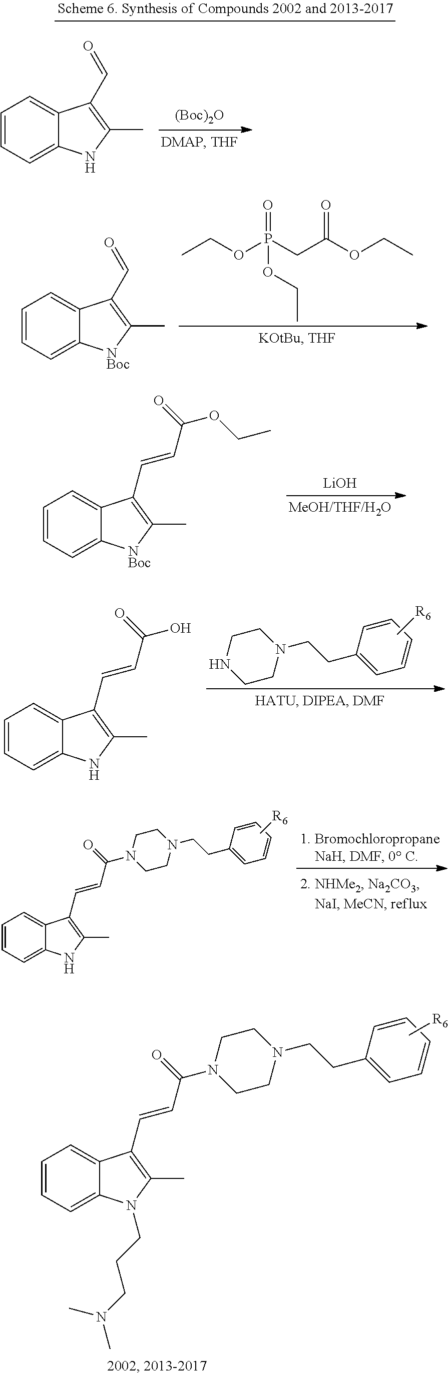

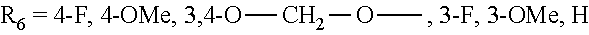

### Preparation of tert-butyl 3-formyl-2-methyl-1H-indole-1-carboxylate

Boc-anhydride (16.45 g, 75.38 mmol) was added to a stirred solution of 2-methyl-1H-indole-3-carbaldehyde (8.00 g, 50.25 mmol) in THF (180 mL). DMAP (2.45 g, 20.1 mmol) was added portionwise at room temperature. The reaction mass was stirred at room temperature for 2 hours. After complete consumption of the starting material, THF was evaporated under vacuum. The residue was dissolved in EtOAc (100 mL) and washed with water followed by brine solution, and then dried over anhydrous Na₂SO₄. The organic layer was concentrated under reduced pressure to afford the crude compound. The crude compound was purified on 100-200 mesh silica gel eluting with 10% EtOAc in petroleum ether to afford a brown solid (12.0 g, 93%).

¹H NMR (300 MHz, CDCl₃): δ 10.36 (s, 1H), 8.38-8.26 (m, 1H), 8.09-8.00 (m, 1H), 7.39-7.27 (m, 2H), 2.93 (s, 3H), 1.72 (s, 9H). LCMS: m/z 260.42 [M+H]⁺.

### Preparation of tert-butyl (E)-3-(3-ethoxy-3-oxoprop-1-en-1-yl)-2-methyl-1H-indole-1-carboxylate

Potassium tert-butoxide (7.7 g, 69.49 mmol) was added to a stirred solution of ethyl 2-(diethoxy phosphoryl) acetate (15.5 g, 69.49 mmol) in THF (60 mL) at 0° C. The mixture was allowed to warm to room temperature for 30 minutes. To this reaction, a mixture of tert-butyl 3-formyl-2-methyl-1H-indole-1-carboxylate (12.0 g, 46.3 mmol) in THF (60 mL) was added drop wise at 0° C. and the resulting reaction mixture was stirred at room temperature for 1 hour. After complete consumption of the starting material, cold water was added into the reaction mixture, which was then extracted with ethyl acetate. The organic layer was washed with brine solution, dried over anhydrous Na₂SO₄ and concentrated under reduced pressure to afford the crude product. The crude compound was purified on 100-200 mesh silica gel eluting with 7-10% EtOAc in petroleum ether to obtain a pale yellow liquid (14.0 g, 88%).

¹H NMR (300 MHz, DMSO-ds): δ 8.16-8.07 (m, 1H), 7.92-7.80 (m, 2H), 7.39-7.27 (m, 2H), 6.53 (d, J=15.0 Hz, 1H), 4.22 (q, J=7.6 Hz, 2H), 2.68 (s, 3H), 1.64 (s, 9H), 1.26 (t, J=7.6 Hz, 3H). LCMS: m/z 330.4 [M+H]⁺.

### Preparation of (E)-3-(2-methyl-1H-indol-3-yl)acrylic acid

To a stirred solution of (E)-tert-butyl 3-(3-ethoxy-3-oxoprop-1-enyl)-2-methyl-1H-indole-1-carboxylate (14.0 g, 42.5 mmol) in THF:MeOH:H₂O (250 mL, 1:2:2) was added LiOH.H₂O (17.83 g, 425 mmol) in water (40 mL) at 0° C. The reaction mixture was allowed to stir at room temperature for 16 hours. After complete consumption of the starting material, the reaction mass was concentrated and then partitioned between ethyl acetate and water. The aqueous layer was collected and acidified with 2 N HCl and extracted with ethyl acetate. The organic layer was washed with brine solution, dried over anhydrous Na₂SO₄ and concentrated under reduced pressure to afford an off white solid (2.8 g, 78%).

¹H NMR (400 MHz, DMSO-d₆): δ 11.81 (s, 1H), 11.66 (s, 1H), 7.82-7.81 (m, 2H), 7.37 (dd, J=6.8 Hz, 2.2 Hz, 1H), 7.19-7.08 (m, 2H), 6.23 (d, J=15.2 Hz, 1H). LCMS: m/z 202.3 [M+H]⁺.

### Preparation of (E)-1-(4-(4-fluorophenethyl)piperazin-1-yl)-3-(2-methyl-1H-indol-3-yl)prop-2-en-1-one

To a stirred solution of (E)-3-(2-methyl-1H-indol-3-yl)acrylic acid (0.40 g, 1.98 mmol) in DMF (4 mL), DIPEA (1.28 mL, 9.93 mmol) was added. The mixture was stirred for 10 minutes, followed by the addition of HATU (1.1 g, 2.98 mmol) and stirring for a further 30 minutes. The reaction mass was cooled to 0° C. and 1-(4-fluorophenethyl)piperazine (0.73 g, 2.98 mmol) was added. The reaction was stirred at room temperature for 16 hours. After complete consumption of the starting material, the reaction mixture was poured into ice water and extracted with ethyl acetate. The organic layer was washed with brine solution, dried over anhydrous Na₂SO₄ and concentrated under reduced pressure to afford the crude product. The crude compound was purified on 100-200 mesh silica gel eluting with 3% MeOH in DCM to obtain a brown solid (0.59 g, 84%).

¹H NMR (300 MHz, DMSO-d₆): δ 11.55 (br s, 1H), 7.86 (dd, J=6.9 Hz, 2.1 Hz, 1H), 7.77 (d, J=15.3 Hz, 1H), 7.39-7.22 (m, 3H), 7.19-7.03 (m, 4H), 6.84 (d, J=15.3 Hz, 1H), 3.63 (br s, 4H), 2.79-2.69 (m, 2H), 2.60-2.39 (m, 9H). LCMS: m/z 392.52 [M+H]⁺.

Other analogues prepared by this method:
(E)-1-(4-(4-methoxyphenethyl)piperazin-1-yl)-3-(2-methyl-1H-indol-3-yl)prop-2-en-1-one (74%).
(E)-1-(4-(2-(benzo[d][1,3]dioxol-5-yl)ethyl)piperazin-1-yl)-3-(2-methyl-1H-indol-3-yl)prop-2-en-1-one (72%).
(E)-1-(4-(3-fluorophenethyl)piperazin-1-yl)-3-(2-methyl-1H-indol-3-yl)prop-2-en-1-one (91%).
(E)-1-(4-(3-methoxyphenethyl)piperazin-1-yl)-3-(2-methyl-1H-indol-3-yl)prop-2-en-1-one (73%)
(E)-1-(4-phenethylpiperazin-1-yl)-3-(2-methyl-1H-indol-3-yl)prop-2-en-1-one (77%).

### Preparation of (E)-3-(1-(3-chloropropyl)-2-methyl-1H-indol-3-yl)-1-(4-(4-fluorophenethyl)piperazin-1-yl)prop-2-en-1-one

NaH (117 mg, 2.94 mmol) was added portionwise to a stirred solution of (E)-1-(4-(4-fluorophenethyl)piperazin-1-yl)-3-(2-methyl-1H-indol-3-yl)prop-2-en-1-one (579 mg, 1.47 mmol) in DMF (10 mL) at 0° C. The mixture was allowed to warm to room temperature for 30 minutes. To this bromochloropropane (0.3 mL, 2.94 mmol) was added dropwise at 0° C. and allowed to stir at room temperature for 3 hours. After complete consumption of the starting material, added ice cold water into reaction mixture and extracted with ethyl acetate. The organic layer was washed with brine solution and dried over anhydrous Na₂SO₄ and concentrated under reduced pressure to afford the crude product. The crude compound was purified on 100-200 mesh silica gel eluting with 2% MeOH in DCM to obtain a brown liquid (770 mg, 92%).

¹H NMR (300 MHz, DMSO-d₆): δ 7.95 (dd, J=6.9 Hz, 2.1 Hz, 1H), 7.79 (d, J=15.3 Hz, 1H), 7.53 (dd, J=7.2 Hz, 1.8 Hz, 1H), 7.35-7.04 (m, 6H), 6.87 (d, J=15.3 Hz, 1H), 4.32 (t, J=7.2 Hz, 2H), 3.75-3.57 (m, 6H), 2.80-2.69 (m, 2H), 2.60-2.39 (m, 9H), 2.13 (quintet, J=7.2 Hz, 2H). LCMS: m/z 468.53 [M+H]⁺.

Other analogues prepared by this method:
(E)-3-(1-(3-chloropropyl)-2-methyl-1H-indol-3-yl)-1-(4-(4-methoxyphenethyl)piperazin-1-yl)prop-2-en-1-one (90%).
(E)-1-(4-(2-(benzo[d][1,3]dioxol-5-yl)ethyl)piperazin-1-yl)-3-(1-(3-chloropropyl)-2-methyl-1H-indol-3-yl)prop-2-en-1-one (50%).
(E)-3-(1-(3-chloropropyl)-2-methyl-1H-indol-3-yl)-1-(4-(3-fluorophenethyl)piperazin-1-yl)prop-2-en-1-one (86%).
(E)-3-(1-(3-chloropropyl)-2-methyl-1H-indol-3-yl)-1-(4-(3-methoxyphenethyl)piperazin-1-yl)prop-2-en-1-one (66%).
(E)-3-(1-(3-chloropropyl)-2-methyl-1H-indol-3-yl)-1-(4-phenethylpiperazin-1-yl)prop-2-en-1-one (72%).

### Preparation of Compound 2002, (E)-3-(1-(3-(dimethylamino)propyl)-2-methyl-1H-indol-3-yl)-1-(4-(4-fluorophenethyl)piperazin-1-yl)prop-2-en-1-one

To a stirred solution of (E)-3-(1-(3-chloropropyl)-2-methyl-1H-indol-3-yl)-1-(4-(4-fluorophenethyl)piperazin-1-yl)prop-2-en-1-one (769 mg, 1.643 mmol) in acetonitrile (70 mL), sodium iodide (615 mg, 4.1 mmol) and sodium carbonate (870 mg, 8.21 mmol), followed by N,N-diethylamine (529 mg, 6.57 mmol) were added at room temperature. The reaction mixture was heated to 75° C. for 16 hours. After complete consumption of the starting material, the reaction mixture was cooled to room temperature, diluted with EtOAc (60 mL), washed with water and brine solution, dried over anhydrous Na₂SO₄ and concentrated under reduced pressure to afford the crude product. The crude compound was purified by flash chromatography using 15% MeOH-DCM as an eluent to afford the target compound as a pale yellow sticky liquid (187 mg, 24%).

¹H NMR (300 MHz, CD₃OD): δ 7.99 (d, J=15.3 Hz, 1H), 7.86 (dd, J=6.0 Hz, 2.1 Hz, 1H), 7.46 (dd, J=6.0 Hz, 1.8 Hz, 1H), 7.27-7.17 (m), 7.00 (t, J=9.0 Hz, 2H), 6.90 (d, J=15.3 Hz, 1H), 4.26 (t, J=7.2 Hz, 2H), 3.79 (br s, 4H), 2.86-2.81 (m, 2H), 2.67-2.58 (m, 9H), 2.41 (t, J=7.2 Hz, 2H), 2.27 (s, 6H), 1.96 (quintet, J=7.2 Hz, 2H). LCMS: m/z 477.5 [M+H]⁺.

Other analogues prepared by this method:

Compound 2013, (E)-3-(1-(3-(dimethylamino)propyl)-2-methyl-1H-indol-3-yl)-1-(4-(4-methoxyphenethyl)piperazin-1-yl)prop-2-en-1-one (31%).

¹H NMR (300 MHz, CD₃OD): δ 7.99 (d, J=15.6 Hz, 1H), 7.86 (d, J=6.9 Hz, 1H), 7.46 (d, J=6.9 Hz, 1H), 7.26-7.18 (m, 2H), 7.14 (d, J=7.5 Hz, 2H), 6.90 (d, J=15.3 Hz, 1H), 6.84 (d, J=8.4 Hz, 2H), 4.26 (t, J=6.9 Hz, 2H), 3.80 (br s, 4H), 3.76 (s, 3H), 2.84-2.73 (m, 2H), 2.69-2.57 (m, 9H), 2.42 (t, J=6.9 Hz, 2H), 2.28 (s, 6H), 2.02-1.93 (m, 2H). LCMS: m/z 489.5 [M+H]⁺.

Compound 2014, (E)-1-(4-(2-(benzo[d][1,3]dioxol-5-yl)ethyl)piperazin-1-yl)-3-(1-(3-(dimethylamino)propyl)-2-methyl-1H-indol-3-yl)prop-2-en-1-one (47%).

¹H NMR (400 MHz, CD₃OD): δ 7.99 (d, J=15.2 Hz), 7.86 (dd, J=6.8 Hz, 2.0 Hz, 1H), 7.46 (dd, J=7.6 Hz, 1.2 Hz), 7.25-7.17 (m, 2H), 6.90 (d, J=15.2 Hz, 1H), 6.75-6.66 (m, 3H), 5.89 (s, 2H), 4.27 (t, J=7.6 Hz, 2H), 3.79 (br s, 4H), 2.79-2.74 (m, 2H), 2.65-2.58 (m, 9H), 2.50 (t, J=7.2 Hz, 2H), 2.33 (s, 6H), 1.99 (quintet, J=7.5 Hz, 2H). LCMS: m/z 503.47 [M+H]⁺.

Compound 2015, (E)-3-(1-(3-(dimethylamino)propyl)-2-methyl-1H-indol-3-yl)-1-(4-(3-fluorophenethyl)piperazin-1-yl)prop-2-en-1-one (23%).

¹H NMR (300 MHz, CD₃OD): δ 7.99 (d, J=15.0 Hz, 1H), 7.86 (dd, J=6.6 Hz, 2.1 Hz, 1H), 7.46 (dd, J=6.9 Hz, 1.8 Hz, 1H), 7.32-7.16 (m, 3H), 7.06 (br d, J=7.8 Hz, 1H), 7.00 (br d, 10.2 Hz, 1H), 6.94-6.87 (m, 2H), 4.26 (t, J=7.2 Hz, 2H), 3.79 (br s, 4H), 2.90-2.84 (m, 2H), 2.70-2.58 (m, 9H), 2.42 (t, J=7.2 Hz, 2H), 2.27 (s, 6H), 1.97 (quintet, J=7.5 Hz, 2H). LCMS: m/z 477.57 [M+H]⁺.

Compound 2016, (E)-3-(1-(3-(dimethylamino)propyl)-2-methyl-1H-indol-3-yl)-1-(4-(3-methoxyphenethyl)piperazin-1-yl)prop-2-en-1-one (33%).

¹H NMR (300 MHz, CD₃OD): δ 7.99 (d, J=15.0 Hz, 1H), 7.90-7.83 (m, 1H), 7.50-7.42 (m, 1H), 7.28-7.14 (m, 3H), 6.95-6.72 (m, 4H), 4.26 (t, J=6.9 Hz, 2H), 3.85-3.72 (m, 7H), 2.88-2.79 (m, 2H), 2.71-2.56 (m, 9H), 2.40 (t, J=6.6 Hz, 2H), 2.26 (s, 6H), 2.05-1.90 (m, 2H). LCMS: m/z 489.6 [M+H]⁺.

Compound 2017, (E)-3-(1-(3-(dimethylamino)propyl)-2-methyl-1H-indol-3-yl)-1-(4-phenethylpiperazin-1-yl)prop-2-en-1-one (38%).

¹H NMR (300 MHz, CD₃OD): δ 7.99 (d, J=15.0 Hz, 1H), 7.86 (dd, J=6.0 Hz, 2.4 Hz, 1H), 7.46 (dd, J=6.3 Hz, 2.4 Hz, 1H), 7.34-7.23 (m, 7H), 6.90 (d, J=15.3 Hz, 1H), 4.26 (t, J=7.2 Hz, 2H), 3.80 (br s, 4H), 2.88-2.82 (m, 2H), 2.70-2.58 (m, 2H), 2.70-2.58 (m, 9H), 2.43 (s, 6H), 2.03-1.91 (m, 2H).

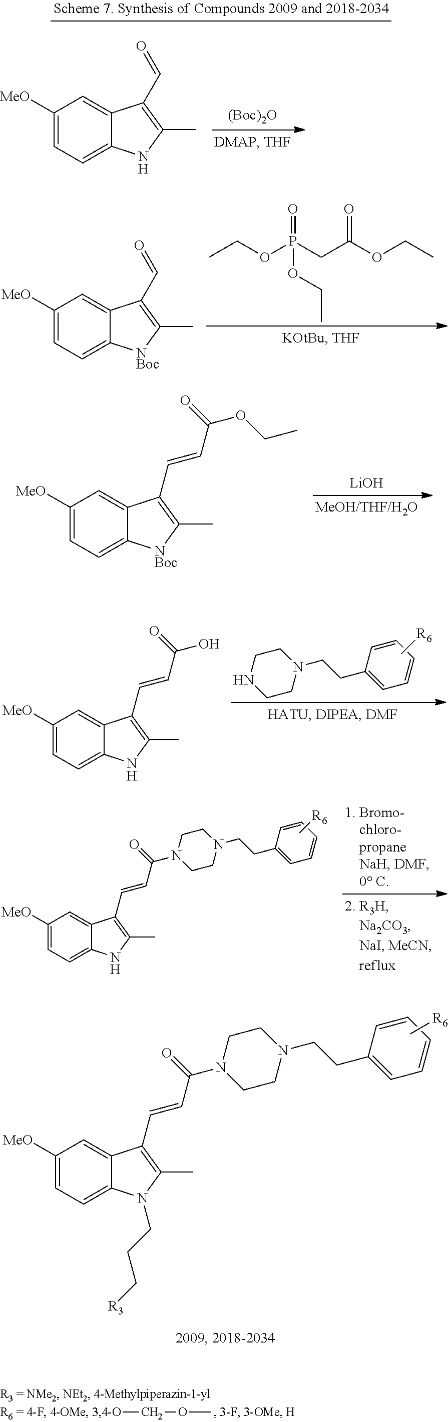

### Preparation of tert-butyl 3-formyl-5-methoxy-2-methyl-1H-indole-1-carboxylate

Boc-anhydride (23.06 g, 105.6 mmol) was added to a stirred solution of 5-methoxy-2-methyl-1H-indole-3-carbaldehyde (10.0 g, 52.95 mmol) in THF (100 mL). DMAP (2.58 g, 21.14 mmol) was then added portionwise at room temperature. The reaction mass was stirred at room temperature for 16 hours. After complete consumption of the starting material based on TLC, THF was evaporated under vacuum. The residue was dissolved in EtOAc (100 mL) and washed with water followed by brine solution, then dried over anhydrous Na₂SO₄. The organic layer was concentrated under reduced pressure to afford the crude product. The crude compound was purified on 100-200 mesh silica gel eluting with 10% EtOAc in petroleum ether to obtain an off white solid (13.0 g, 86%).

¹H NMR (300 MHz, DMSO-d₆): δ 10.28 (s, 1H), 7.95 (d, J=8.7 Hz, 1H), 7.68 (d, J=2.4 Hz, 1H), 6.96 (dd, J=8.7 Hz, 2.4 Hz, 1H), 3.79 (s, 3H), 2.87 (s, 3H), 1.62 (s, 9H). LCMS: m/z 289.18 [M+H]⁺.

### Preparation of tert-butyl (E)-3-(3-ethoxy-3-oxoprop-1-en-1-yl)-5-methoxy-2-methyl-1H-indole-1-carboxylate

Potassium tert-butoxide (5.8 g, 51.84 mmol) was added to a stirred solution of ethyl 2-(diethoxyphosphoryl)acetate (11.6 g, 51.84 mmol) in THF (75 mL) at 0° C. and allowed to warm to room temperature for 30 minutes. To this reaction mixture tert-butyl 3-formyl-5-methoxy-2-methyl-1H-indole-1-carboxylate (10.0 g, 34.56 mmol) in THF (75 mL) was added drop wise at 0° C. and the resulting reaction mixture was stirred at room temperature for 3 hours. After complete consumption of the starting material, cold water was added into the reaction mixture, which was then extracted with ethyl acetate. The organic layer was washed with brine solution, dried over anhydrous Na₂SO₄ and concentrated under reduced pressure to afford the crude product. The crude compound was purified on 100-200 mesh silica gel eluting with 5% EtOAc in petroleum ether to obtain a white solid (8.0 g, 66%).

¹H NMR (300 MHz, CDCl₃): δ 8.02 (d, J=8.7 Hz, 1H), 7.95 (d, J=15.0 Hz, 1H), 7.24 (d, J=2.4 Hz, 1H), 6.89 (dd, J=8.7 Hz, 2.4 Hz, 1H), 6.44 (d, J=15.0 Hz, 1H), 4.27 (q, J=7.6 Hz, 2H), 3.86 (s, 3H), 2.68 (s, 3H), 1.64 (s, 9H), 1.37 (t, J=7.6 Hz, 3H). LCMS: m/z 359.40 [M+H]⁺.

### Preparation of (E)-3-(5-methoxy-2-methyl-1H-indol-3-yl)acrylic acid

tert-Butyl (E)-3-(3-ethoxy-3-oxoprop-1-en-1-yl)-5-methoxy-2-methyl-1H-indole-1-carboxylate (6.0 g, 16.71 mmol) was dissolved in THF:MeOH (120 mL, 1:1). To this solution LiOH.H₂O (7.0 g, 167.1 mmol) in water (70 mL) was added at 0° C. The reaction mixture was allowed to stir at room temperature for 16 hours. After complete consumption of the starting material, the reaction mass was concentrated and then partitioned between ethyl acetate and water. The aqueous layer was collected, acidified with 2 N HCl and extracted with ethyl acetate. The organic layer was washed with brine solution, dried over anhydrous Na₂SO₄ and concentrated under reduced pressure to afford a grey solid (3.6 g, 94%).

¹H NMR (300 MHz, DMSO-d₆): δ 11.79 (br s, 1H), 11.52 (br s, 1H), 7.78 (d, J=15.0 Hz, 1H), 7.25 (d, J=8.7 Hz, 1H), 7.18 (d, J=2.4 Hz, 1H), 6.77 (dd, J=8.7 Hz, 2.4 Hz, 1H), 6.14 (d, J=15.0 Hz, 1H), 3.81 (s, 3H), 2.48 (s, 3H). LCMS: m/z 231.1 [M+H]⁺.

### Preparation of (E)-3-(5-methoxy-2-methyl-1H-indol-3-yl)-1-(4-phenethylpiperazin-1-yl)prop-2-en-1-one

To a stirred solution of (E)-3-(5-methoxy-2-methyl-1H-indol-3-yl)acrylic acid (1.2 g, 5.19 mmol) in DMF (15 mL), DIPEA (3.44 mL, 20.77 mmol) was added. The mixture was stirred for 10 minutes, followed by the addition of HATU (3.95 mg, 10.38 mmol) and stirring for a further 30 minutes. The reaction mass cooled to 0° C. and 1-phenethylpiperazine (1.18 g, 6.23 mmol) was added. The reaction mixture was stirred at room temperature for 16 hours. After complete consumption of the starting material, the reaction mixture was poured into ice water and extracted with ethyl acetate. The organic layer was washed with brine solution, dried over anhydrous Na₂SO₄ and concentrated under reduced pressure to afford the crude product. The crude compound was purified on 100-200 mesh silica gel eluting with 5% MeOH in DCM to obtain a yellow solid (1.3 g, 65%).

¹H NMR (300 MHz, DMSO-d₆): δ 11.41 (br s, 1H), 7.74 (d, J=15.0 Hz, 1H), 7.34-7.12 (m, 7H), 6.91-6.68 (m, 2H), 3.81 (s, 3H), 3.62 (br s, 4H), 2.81-2.70 (m, 2H), 2.68-2.32 (m, 9H).

Other analogues prepared by this method:
(E)-3-(5-methoxy-2-methyl-1H-indol-3-yl)-1-(4-(4-fluorophenethyl)piperazin-1-yl)prop-2-en-1-one (58%).
(E)-3-(5-methoxy-2-methyl-1H-indol-3-yl)-1-(4-(4-methoxyphenethyl)piperazin-1-yl)prop-2-en-1-one (55%).
(E)-1-(4-(2-(benzo[d][1,3]dioxol-5-yl)ethyl)piperazin-1-yl)-3-(5-methoxy-2-methyl-1H-indol-3-yl)prop-2-en-1-one (35%).
(E)-3-(5-methoxy-2-methyl-1H-indol-3-yl)-1-(4-(3-fluorophenethyl)piperazin-1-yl)prop-2-en-1-one (53%).
(E)-3-(5-methoxy-2-methyl-1H-indol-3-yl)-1-(4-(3-methoxyphenethyl)piperazin-1-yl)prop-2-en-1-one (46%).

### Preparation of (E)-3-(1-(3-chloropropyl)-5-methoxy-2-methyl-1H-indol-3-yl)-1-(4-(4-fluorophenethyl)piperazin-1-yl)prop-2-en-1-one

NaH (100 mg, 2.37 mmol) was added portion wise to a stirred solution of (E)-3-(5-methoxy-2-methyl-1H-indol-3-yl)-1-(4-(4-fluorophenethyl)piperazin-1-yl)prop-2-en-1-one (500 mg, 1.18 mmol) in DMF (10 mL) at 0° C. The mixture was allowed to warm to room temperature for 30 minutes. To this bromochloropropane (6.0 mL, 1.18 mmol) was added dropwise at 0° C. The reaction mixture was allowed to warm to room temperature and was stirred for 3 hours. After complete consumption of the starting material, ice cold water was added to the reaction mixture and extracted with ethyl acetate. The organic layer was washed with brine solution, dried over anhydrous Na₂SO₄ and concentrated under reduced pressure to afford the crude product. The crude compound was purified on 100-200 mesh silica gel eluting with 5% MeOH in DCM to obtain a brown solid (300 mg, 51%).

¹H NMR (400 MHz, CD₃OD): δ 7.98 (d, J=15.2 Hz, 1H), 7.39 (d, J=8.8 Hz, 1H), 7.31 (d, J=2.4 Hz, 1H), 7.23 (dd, J=8.4 Hz, 5.4 Hz, 2H), 7.00 (t, J=8.8 Hz, 2H), 6.89 (dd, J=8.8 Hz, 2.4 Hz, 1H), 6.78 (d, J=15.2 Hz, 1H), 4.33 (t, J=7.2 Hz, 2H), 3.83 (s, 3H), 3.78 (br s, 4H), 3.72 (t, J=7.2 Hz, 2H), 2.93-2.79 (m, 2H), 2.69-2.59 (m, 6H), 2.58 (s, 3H), 2.27-2.19 (m, 2H). LCMS: m/z 498.36 [M+H]⁺.

Other analogues prepared by this method:
(E)-3-(1-(3-chloropropyl)-5-methoxy-2-methyl-1H-indol-3-yl)-1-(4-(4-methoxyphenethyl)piperazin-1-yl)prop-2-en-1-one (83%).
(E)-1-(4-(2-(benzo[d][1,3]dioxol-5-yl)ethyl)piperazin-1-yl)-3-(1-(3-chloropropyl)-5-methoxy-2-methyl-1H-indol-3-yl)prop-2-en-1-one (100%).
(E)-3-(1-(3-chloropropyl)-5-methoxy-2-methyl-1H-indol-3-yl)-1-(4-(3-fluorophenethyl)piperazin-1-yl)prop-2-en-1-one (86%).
(E)-3-(1-(3-chloropropyl)-5-methoxy-2-methyl-1H-indol-3-yl)-1-(4-(3-methoxyphenethyl)piperazin-1-yl)prop-2-en-1-one (91%).
(E)-3-(1-(3-chloropropyl)-5-methoxy-2-methyl-1H-indol-3-yl)-1-(4-phenethylpiperazin-1-yl)prop-2-en-1-one (47%).

### Preparation of Compound 2023, (E)-3-(1-(3-(diethylamino)propyl)-5-methoxy-2-methyl-1H-indol-3-yl)-1-(4-(4-fluorophenethyl)piperazin-1-yl)prop-2-en-1-one

To a stirred solution of (E)-3-(1-(3-chloropropyl)-2-methyl-1H-indol-3-yl)-1-(4-(4-fluorophenethyl)piperazin-1-yl)prop-2-en-1-one (1.08 g, 2.25 mmol) in acetonitrile (10 mL) at room temperature, sodium iodide (180 mg, 1.20 mmol) and sodium carbonate (319 mg, 3.01 mmol) were added, followed by N,N-diethylamine (0.195 mL, 2.41 mmol). The reaction mixture was heated to 75° C. for 16 hours. After complete consumption of the starting material, the reaction mixture was cooled to room temperature, diluted with EtOAc (60 mL), washed with water and brine solution, dried over anhydrous Na₂SO₄ and concentrated under reduced pressure to afford the crude product. The crude compound was purified by flash column chromatography on silica gel to afford the target compound as pale yellow gummy solid (85 mg, 26%).

¹H NMR (300 MHz, CD₃OD): δ 7.97 (d, J=15.0 Hz, 1H), 7.38 (d, J=9.0 Hz, 1H), 7.31 (d, J=2.4 Hz, 1H), 7.24 (dd, J=8.7 Hz, 5.7 Hz, 2H), 7.00 (t, J=8.7 Hz, 2H), 6.89 (dd, J=2.1 Hz, 8.7 Hz, 1H), 6.80 (d, J=15.0 Hz, 1H), 4.23 (t, J=6.9 Hz, 2H), 3.87 (s, 3H), 3.79 (br s, 4H), 2.87-2.80 (m, 2H), 2.70-2.58 (m, 12H), 2.57 (s, 3H), 2.03-1.96 (m, 2H), 1.04 (t, J=7.2 Hz, 6H). LCMS: m/z 535.55 [M+H]⁺.

Other analogues prepared by this method:

Compound 2009, (E)-3-(1-(3-(dimethylamino)propyl)-5-methoxy-2-methyl-1H-indol-3-yl)-1-(4-phenethylpiperazin-1-yl)prop-2-en-1-one (35%).

¹H NMR (400 MHz, CD₃OD): δ 7.97 (d, J=15.2 Hz, 1H), 7.36 (d, J=8.0 Hz, 1H), 7.30 (d, J=2.4 Hz, 1H), 7.28-7.15 (m, 5H), 6.89 (dd, J=8.8 Hz, 2.4 Hz, 1H), 6.79 (d, J=15.2 Hz, 1H), 4.21 (t, J=7.6 Hz, 2H), 3.91 (s, 3H), 3.79 (br s, 4H), 2.87-2.83 (m, 2H), 2.68-2.60 (m, 6H), 2.56 (s, 3H), 2.35 (t, J=7.6 Hz, 2H), 2.23 (s, 6H), 1.93 (quintet, J=7.6 Hz, 2H). LCMS: m/z 489.5 [M+H]⁺.

Compound 2018, (E)-3-(1-(3-(dimethylamino)propyl)-5-methoxy-2-methyl-1H-indol-3-yl)-1-(4-(4-fluorophenethyl)piperazin-1-yl)prop-2-en-1-one (44%).

¹H NMR (300 MHz, CD₃OD): δ 7.97 (d, J=15.0 Hz, 1H), 7.37 (d, J=8.7 Hz, 1H), 7.30 (d, J=2.4 Hz, 1H), 7.24 (dd, J=8.4 Hz, 5.4 Hz, 2H), 7.00 (t, J=8.7 Hz, 2H), 6.89 (dd, J=8.7 Hz, 2.1 Hz, 1H), 6.79 (d, J=15.3 Hz, 1H), 4.22 (t, J=7.5 Hz, 2H), 3.87 (s, 3H), 3.84-3.75 (m, 4H), 2.87-2.70 (m, 2H), 2.69-2.58 (m, 6H), 2.56 (s, 3H), 2.38-2.33 (m, 2H), 2.24 (s, 6H), 1.94 (quintet, J=7.2 Hz, 2H). LCMS: m/z 507.5 [M+H]⁺.

Compound 2019, (E)-3-(1-(3-(dimethylamino)propyl)-5-methoxy-2-methyl-1H-indol-3-yl)-1-(4-(4-methoxyphenethyl)piperazin-1-yl)prop-2-en-1-one (16%).

¹H NMR (400 MHz, CD₃OD): δ 7.97 (d, J=15.2 Hz, 1H), 7.37 (d, J=9.2 Hz, 1H), 7.31 (d, J=2.4 Hz, 1H), 7.14 (d, J=8.8 Hz, 2H), 6.90 (dd, J=9.2 Hz, 2.0 Hz, 1H), 6.84 (d, J=8.8 Hz, 2H), 4.24 (t, J=7.2 Hz, 2H), 3.87 (s, 3H), 3.79 (br s, 4H), 2.81-2.76 (m, 2H), 2.67-2.59 (m, 11H), 2.41 (s, 6H), 2.00 (quintet, J=7.2 Hz, 2H). LCMS: m/z 519.5 [M+H]⁺.

Compound 2020, (E)-1-(4-(2-(benzo[d][1,3]dioxol-5-yl)ethyl)piperazin-1-yl)-3-(1-(3-(dimethylamino)propyl)-5-methoxy-2-methyl-1H-indol-3-yl)prop-2-en-1-one (13%).

¹H NMR (400 MHz, CD₃OD): δ 7.97 (d, J=15.2 Hz, 1H), 7.37 (d, J=8.8 Hz, 1H), 7.30 (d, J=2.8 Hz, 1H), 6.89 (dd, J=8.8 Hz, 2.4 Hz, 1H), 6.79 (d, J=15.2 Hz, 1H), 6.74-6.67 (m, 3H), 5.89 (s, 2H), 4.21 (t, J=6.8 Hz, 2H), 3.87 (s, 3H), 3.78 (br s, 4H), 2.79-2.74 (m, 2H), 2.66-2.58 (m, 6H), 2.56 (s, 3H), 2.36 (br t, J=6.8 Hz, 2H), 2.23 (s, 6H), 1.94 (quintet, J=7.2 Hz, 2H). LCMS: m/z 533.4 [M+H]⁺.

Compound 2021, (E)-3-(1-(3-(dimethylamino)propyl)-5-methoxy-2-methyl-1H-indol-3-yl)-1-(4-(3-fluorophenethyl)piperazin-1-yl)prop-2-en-1-one (28%).

¹H NMR (400 MHz, CD₃OD): δ 7.97 (d, J=15.2 Hz, 1H), 7.36 (d, J=8.8 Hz, 1H), 7.31-7.26 (m, 2H), 7.05 (br d, J=7.6 Hz, 1H), 6.99 (br d, J=10.0 Hz, 1H), 6.94-6.86 (m, 2H), 6.79 (d, J=15.2 Hz, 1H), 4.21 (t, J=7.6 Hz, 2H), 3.87 (s, 3H), 3.78 (br s, 4H), 2.89-2.84 (m, 2H), 2.69-2.57 (m, 6H), 2.56 (s, 3H), 2.38-2.31 (m, 2H), 2.24 (s, 6H), 1.93 (quintet, J=7.2 Hz, 2H). LCMS: m/z 507.5 [M+H]⁺.

Compound 2022, (E)-3-(1-(3-(dimethylamino)propyl)-5-methoxy-2-methyl-1H-indol-3-yl)-1-(4-(3-methoxyphenethyl)piperazin-1-yl)prop-2-en-1-one (27%).

¹H NMR (300 MHz, CD₃OD): δ 7.97 (d, J=15.0 Hz, 1H), 7.37 (d, J=9.3 Hz, 1H), 7.30 (d, J=2.4 Hz, 1H), 7.18 (t, J=8.1 Hz, 1H), 6.89 (dd, J=9.0 Hz, 2.4 Hz, 1H), 6.83-6.72 (m, 4H), 4.21 (t, J=7.2 Hz, 2H), 3.87 (s, 3H), 3.81-3.75 (m, 7H), 2.86-2.78 (m, 2H), 2.70-2.58 (m, 6H), 2.56 (s, 3H), 2.36 (br t, J=6.9 Hz, 2H), 2.24 (s, 6H), 1.94 (quintet, J=7.2 Hz, 2H). LCMS: m/z 519.58 [M+H]⁺.

Compound 2024, (E)-3-(1-(3-(diethylamino)propyl)-5-methoxy-2-methyl-1H-indol-3-yl)-1-(4-(4-methoxyphenethyl)piperazin-1-yl)prop-2-en-1-one (26%).

¹H NMR (300 MHz, CD₃OD): δ 7.97 (d, J=15.0 Hz, 1H), 7.37 (d, J=8.7 Hz, 1H), 7.30 (br s, 1H), 7.14 (d, J=8.1 Hz, 2H), 6.92-6.74 (m, 4H), 4.21 (t, J=6.6 Hz, 2H), 3.87 (s, 3H), 3.79-3.73 (m, 7H), 2.81-2.73 (m, 2H), 2.69-2.45 (m, 15H), 1.99-1.85 (m, 2H), 1.00 (t, J=7.2 Hz, 6H). LCMS: m/z 547.53 [M+H]⁺.

Compound 2025, (E)-1-(4-(2-(benzo[d][1,3]dioxol-5-yl)ethyl)piperazin-1-yl)-3-(1-(3-(diethylamino)propyl)-5-methoxy-2-methyl-1H-indol-3-yl)prop-2-en-1-one (12%).

¹H NMR (400 MHz, CD₃OD): δ 7.97 (d, J=15.2 Hz, 1H), 7.37 (d, J=8.8 Hz, 1H), 7.30 (d, J=2.0 Hz, 1H), 6.89 (dd, J=8.8 Hz, 2.8 Hz, 1H), 6.79 (d, J=15.2 Hz, 1H), 6.74-6.67 (m, 3H), 5.89 (s, 2H), 4.21 (t, J=7.2 Hz, 2H), 3.87 (s, 3H), 3.78 (br s, 4H), 2.79-2.74 (m, 2H), 2.66-2.52 (m, 15H), 1.93 (quintet, J=7.6 Hz, 2H), 1.01 (t, J=7.2 Hz, 6H). LCMS: m/z 561.4 [M+H]⁺.

Compound 2026, (E)-3-(1-(3-(diethylamino)propyl)-5-methoxy-2-methyl-1H-indol-3-yl)-1-(4-(3-fluorophenethyl)piperazin-1-yl)prop-2-en-1-one (60%).

¹H NMR (400 MHz, CD₃OD): δ 7.97 (d, J=15.6 Hz, 1H), 7.37 (d, J=8.8 Hz, 1H), 7.31-7.24 (m, 2H), 7.05 (br d, J=8.0 Hz, 1H), 7.00 (br d, J=10.0 Hz, 1H), 6.95-6.87 (m, 2H), 6.79 (d, J=15.2 Hz, 1H), 4.21 (t, J=7.2 Hz, 2H), 3.87 (s, 3H), 3.79 (br s, 4H), 2.89-2.84 (m, 2H), 2.69-2.52 (m, 15H), 1.94 (quintet, J=7.2 Hz, 2H), 1.01 (t, J=7.6 Hz, 6H). LCMS: m/z 535.5 [M+H]⁺.

Compound 2027, (E)-3-(1-(3-(diethylamino)propyl)-5-methoxy-2-methyl-1H-indol-3-yl)-1-(4-(3-methoxyphenethyl)piperazin-1-yl)prop-2-en-1-one (10%).

¹H NMR (300 MHz, CD₃OD): δ 7.97 (d, J=15.0 Hz, 1H), 7.37 (d, J=8.7 Hz, 1H), 7.30 (d, J=2.1 Hz, 1H), 7.18 (t, J=7.5 Hz, 1H), 6.89 (dd, J=8.7 Hz, 2.4 Hz, 1H), 6.83-6.72 (m, 4H), 4.21 (t, J=6.9 Hz, 2H), 3.87 (s, 3H), 3.83-3.73 (m, 7H), 2.86-2.78 (m, 2H), 2.69-2.50 (m, 15H), 1.93 (quintet, J=7.8 Hz, 2H), 1.00 (t, J=7.5 Hz, 6H). LCMS: m/z 547.6 [M+H]⁺.

Compound 2028, (E)-3-(1-(3-(diethylamino)propyl)-5-methoxy-2-methyl-1H-indol-3-yl)-1-(4-phenethylpiperazin-1-yl)prop-2-en-1-one (31%).

¹H NMR (400 MHz, CD₃OD): δ 7.97 (d, J=15.2 Hz, 1H), 7.37 (d, J=8.8 Hz, 1H), 7.30 (d, J=2.0 Hz, 1H), 7.27-7.20 (m, 4H), 7.17 (t, J=6.8 Hz, 1H), 6.89 (dd, J=8.8 Hz, 2.8 Hz, 1H), 6.79 (d, J=15.2 Hz, 1H), 4.21 (t, J=7.2 Hz, 2H), 3.87 (s, 3H), 3.79 (br s, 4H), 2.88-2.82 (m, 2H), 2.68-2.50 (m, 15H), 1.93 (quintet, J=7.6 Hz, 2H), 1.00 (t, J=7.6 Hz, 6H). LCMS: m/z 517.58 [M+H]⁺.

Compound 2029, (E)-1-(4-(4-fluorophenethyl)piperazin-1-yl)-3-(5-methoxy-2-methyl-1-(3-(4-methylpiperazin-1-yl)propyl)-1H-indol-3-yl)prop-2-en-1-one (20%).

¹H NMR (300 MHz, CD₃OD): δ 7.97 (d, J=15.0 Hz, 1H), 7.38 (d, J=8.7 Hz, 1H), 7.29 (d, J=2.4 Hz, 1H), 7.24 (dd, J=8.4 Hz, 5.7 Hz, 2H), 6.99 (t, J=8.7 Hz, 2H), 6.88 (dd, J=8.7 Hz, 2.7 Hz, 1H), 6.78 (d, J=15.6 Hz, 1H), 4.23 (t, J=7.2 Hz, 2H), 3.87 (s, 3H), 3.78 (br s, 4H), 2.88-2.70 (m, 2H), 2.69-2.25 (m, 22H), 1.95 (quintet, J=6.9 Hz, 2H). LCMS: m/z 562.51 [M+H]⁺.

Compound 2030, (E)-3-(5-methoxy-2-methyl-1-(3-(4-methylpiperazin-1-yl)propyl)-1H-indol-3-yl)-1-(4-(4-methoxyphenethyl)piperazin-1-yl)prop-2-en-1-one (29%).

¹H NMR (300 MHz, CD₃OD): δ 7.97 (d, J=15.3 Hz, 1H), 7.38 (d, J=8.4 Hz, 1H), 7.29 (br s, 1H), 7.14 (d, J=8.7 Hz, 2H), 6.92-6.73 (m, 4H), 4.24 (t, J=6.6 Hz, 2H), 3.87 (s, 3H), 3.83-3.72 (m, 7H), 2.82-2.73 (m, 2H), 2.69-2.28 (m, 22H), 2.00-1.89 (m, 2H). LCMS: m/z 574.53 [M+H]⁺.

Compound 2031, (E)-1-(4-(2-(benzo[d][1,3]dioxol-5-yl)ethyl)piperazin-1-yl)-3-(5-methoxy-2-methyl-1-(3-(4-methylpiperazin-1-yl)propyl)-1H-indol-3-yl)prop-2-en-1-one (10%).

¹H NMR (400 MHz, CD₃OD): δ 7.97 (d, J=15.2 Hz, 1H), 7.38 (d, J=8.8 Hz, 1H), 7.29 (d. J=2.4 Hz, 1H), 6.88 (dd, J=8.8 Hz, 2.0 Hz, 1H), 6.78 (d, J=15.2 Hz, 1H), 6.75-6.66 (m, 3H), 5.89 (s, 2H), 4.23 (t, J=6.8 Hz, 2H), 3.87 (s, 3H), 3.78 (br s, 4H), 2.79-2.74 (m, 2H), 2.69-2.24 (m, 22H), 1.95 (quintet, J=7.2 Hz, 2H). LCMS: m/z 588.5 [M+H]⁺.

Compound 2032, (E)-1-(4-(3-fluorophenethyl)piperazin-1-yl)-3-(5-methoxy-2-methyl-1-(3-(4-methylpiperazin-1-yl)propyl)-1H-indol-3-yl)prop-2-en-1-one (14%).

¹H NMR (400 MHz, CD₃OD): δ 7.97 (d, J=15.2 Hz, 1H), 7.38 (d, J=8.8 Hz, 1H), 7.32-7.24 (m, 2H), 7.05 (br d, J=8.0 Hz, 1H), 7.00 (br d, J=10.0 Hz, 1H), 6.95-6.85 (m, 2H), 6.78 (d, J=15.2 Hz, 1H), 4.23 (t, J=6.8 Hz, 2H), 3.87 (s, 3H), 3.78 (br s, 4H), 2.90-2.83 (m, 2H), 2.72-2.24 (m, 22H), 1.95 (quintet, J=6.8 Hz, 2H). LCMS: m/z 562.4 [M+H]⁺.

Compound 2033, (E)-3-(5-methoxy-2-methyl-1-(3-(4-methylpiperazin-1-yl)propyl)-1H-indol-3-yl)-1-(4-(3-methoxyphenethyl)piperazin-1-yl)prop-2-en-1-one (22%).

¹H NMR (300 MHz, CD₃OD): δ 7.97 (d, J=15.0 Hz, 1H), 7.38 (d, J=8.7 Hz, 1H), 7.30 (d, J=2.1 Hz, 1H), 7.18 (t, J=8.1 Hz, 1H), 6.88 (dd, J=8.7 Hz, 2.1 Hz, 1H), 6.84-6.72 (m, 4H), 4.23 (t, J=7.2 Hz, 2H), 3.87 (s, 3H), 3.83-3.73 (m, 7H), 2.87-2.79 (m, 2H), 2.70-2.26 (m, 22H), 1.95 (quintet, J=6.9 Hz, 2H). LCMS: m/z 574.6 [M+H]⁺.

Compound 2034, (E)-3-(5-methoxy-2-methyl-1-(3-(4-methylpiperazin-1-yl)propyl)-1H-indol-3-yl)-1-(4-phenethylpiperazin-1-yl)prop-2-en-1-one (22%).

¹H NMR (400 MHz, CD₃OD): δ 7.97 (d, J=15.2 Hz, 1H), 7.38 (d, J=8.8 Hz, 1H), 7.30 (d, J=2.4 Hz, 1H), 7.28-7.21 (m, 4H), 7.17 (t, J=6.8 Hz, 1H), 6.88 (dd, J=8.8 Hz, 2.8 Hz, 1H), 6.79 (d, J=15.2 Hz, 1H), 4.23 (t, J=7.2 Hz, 2H), 3.87 (s, 3H), 3.79 (br s, 4H), 2.88-2.82 (m, 2H), 2.69-2.36 (m, 17H), 2.34 (t, J=6.8 Hz, 2H), 2.28 (s, 3H), 1.95 (quintet, J=6.8 Hz, 2H). LCMS: m/z 544.4 [M+H]⁺.

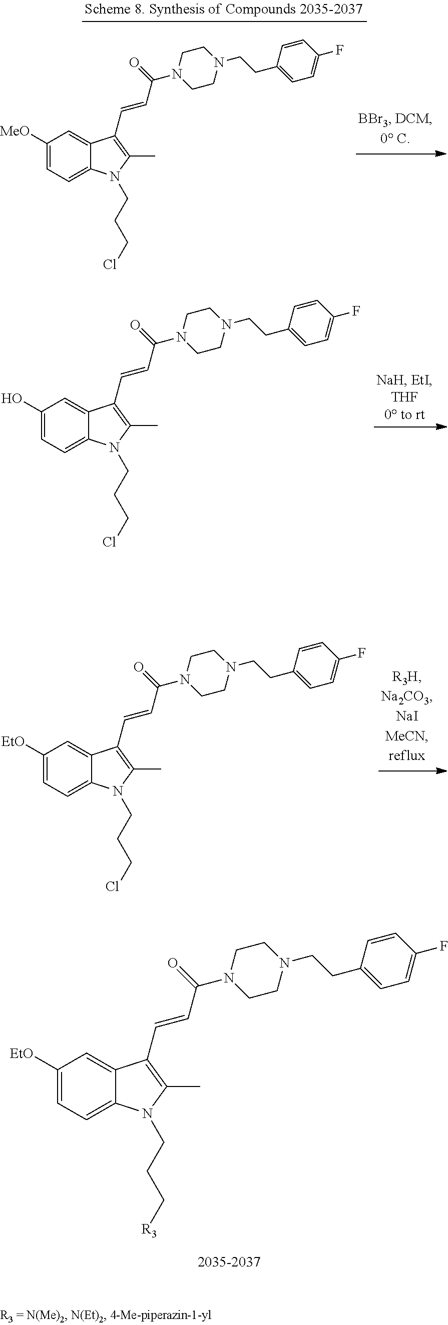

### Preparation of (E)-3-(1-(3-chloropropyl)-5-hydroxy-2-methyl-1H-indol-3-yl)-1-(4-(4-fluorophenethyl)piperazin-1-yl)prop-2-en-1-one

To a solution of (E)-3-(1-(3-chloropropyl)-5-methoxy-2-methyl-1H-indol-3-yl)-1-(4-(4-fluorophenethyl)piperazin-1-yl)prop-2-en-1-one (1.8 g, 3.62 mmol) in dry DCM (50 mL) was added boron tribromide (0.90 mL, 9.3 mmol) at 0° C. The mixture was allowed to warm to room temperature and stirred for 16 hours. After complete consumption of the starting material, the reaction mixture was adjusted to neutral pH using saturated NaHCO₃ solution. The resultant was extracted with 5% MeOH in DCM. The organic layer was washed with brine solution, dried over anhydrous Na₂SO₄ and concentrated under reduced pressure to afford the crude product (1.3 g, 74%), which was used without further purification. LCMS: m/z 484.43 [M+H]⁺.

### Preparation of (E)-3-(1-(3-chloropropyl)-5-ethoxy-2-methyl-1H-indol-3-yl)-1-(4-(4-fluorophenethyl)piperazin-1-yl)prop-2-en-1-one

NaH (161 mg, 6.7 mmol) was added portionwise to a stirred solution of (E)-3-(1-(3-chloropropyl)-5-hydroxy-2-methyl-1H-indol-3-yl)-1-(4-(4-fluorophenethyl)piperazin-1-yl)prop-2-en-1-one (1.3 g, 2.7 mmol) in THF (20 mL) at 0° C. The mixture was allowed to warm to room temperature for 30 minutes. To this, ethyl iodide (0.46 mL, 5.38 mmol) was added dropwise at 0° C. The mixture was allowed to warm to room temperature and was stirred for 6 hours. After complete consumption of the starting material, ice cold water was added and the reaction mixture was extracted with ethyl acetate. The organic layer was washed with brine solution, dried over anhydrous Na₂SO₄ and concentrated under reduced pressure to afford the crude product. The crude compound was purified by flash column chromatography on silica gel to afford a brown sticky solid (600 mg, 43%).

¹H NMR (300 MHz, DMSO-d₆): δ 7.79 (d, J=15.6 Hz, 1H), 7.42 (d, J=8.7 Hz, 1H), 7.32-7.23 (m, 3H), 7.09 (t, J=9.0 Hz, 2H), 6.86 (dd, J=8.7 Hz, 2.1 Hz, 1H), 6.77 (d, J=15.6 Hz, 1H), 4.24 (t, J=7.2 Hz, 2H), 4.10 (q, J=7.5 Hz, 2H), 3.72-3.58 (m, 6H), 2.80-2.69 (m, 2H), 2.60-2.39 (m, 6H), 2.19-2.04 (m, 2H), 1.35 (t, J=7.2 Hz, 3H).

### Preparation of Compound 2035, (E)-3-(1-(3-(dimethylamino)propyl)-5-ethoxy-2-methyl-1H-indol-3-yl)-1-(4-(4-fluorophenethyl)piperazin-1-yl)prop-2-en-1-one

To a stirred solution of (E)-3-(1-(3-chloropropyl)-5-ethoxy-2-methyl-1H-indol-3-yl)-1-(4-(4-fluorophenethyl)piperazin-1-yl)prop-2-en-1-one (190 mg, 0.37 mmol) in acetonitrile (8 mL) at room temperature, sodium iodide (111 mg, 0.74 mmol) and sodium carbonate (196 mg, 1.85 mmol) were added, followed by N,N-dimethylamine hydrochloride (0.133 mL, 1.48 mmol). The reaction mixture was heated to 75° C. for 16 hours. After complete consumption of the starting material, the reaction mixture was cooled to room temperature, diluted with EtOAc (60 mL), washed with water and brine solution, dried over anhydrous Na₂SO₄ and concentrated under reduced pressure to afford the crude product. The crude compound was purified on 230-400 mesh silica gel, eluting with 5% MeOH in DCM to obtained the desired product as a yellow gummy liquid (35 mg, 18%).

¹H NMR (300 MHz, CD₃OD): δ 7.96 (d, J=16.5 Hz, 1H), 7.36 (d, J=8.4 Hz, 1H), 7.31-7.20 (m, 3H), 6.99 (t, J=9.0 Hz, 2H), 6.88 (br d, J=8.4 Hz, 1H), 6.78 (d, J=14.4 Hz, 1H), 4.21 (t, J=6.3 Hz, 2H), 4.11 (q, J=7.2 Hz, 2H), 3.78 (br s, 4H), 2.87-2.80 (m, 2H), 2.69-2.58 (m, 6H), 2.56 (s, 3H), 2.42-2.31 (m, 2H), 2.24 (s, 6H), 2.00-1.88 (m, 2H), 1.42 (t, J=6.6 Hz, 3H). LCMS: m/z 521.53 [M+H]⁺.

Other analogues prepared by this method:

Compound 2036, (E)-3-(1-(3-(diethylamino)propyl)-5-ethoxy-2-methyl-1H-indol-3-yl)-1-(4-(4-fluorophenethyl)piperazin-1-yl)prop-2-en-1-one (24%).

¹H NMR (300 MHz, CD₃OD): δ 7.96 (d, J=14.4 Hz, 1H), 7.36 (d, J=9.6 Hz, 1H), 7.32-7.20 (m, 3H), 7.00 (t, J=8.1 Hz, 2H), 6.89 (br d, J=9.0 Hz, 1H), 6.78 (d, J=16.5 Hz, 1H), 4.21 (t, J=7.5 Hz, 2H), 4.11 (q, J=6.9 Hz, 2H), 3.79 (br s, 4H), 2.87-2.80 (m, 2H), 2.69-2.48 (m, 15H), 1.99-1.87 (m, 2H), 1.42 (t, J=6.9 Hz, 3H), 1.00 (t, J=7.5 Hz, 6H). LCMS: m/z 549.51 [M+H]⁺.

Compound 2037, (E)-3-(5-ethoxy-2-methyl-1-(3-(4-methylpiperazin-1-yl)propyl)-1H-indol-3-yl)-1-(4-(4-fluorophenethyl)piperazin-1-yl)prop-2-en-1-one (17%).

¹H NMR (300 MHz, CD₃OD): δ 7.96 (d, J=15.6 Hz, 1H), 7.37 (d, J=9.3 Hz, 1H), 7.31-7.20 (m, 3H), 7.00 (t, J=8.4 Hz, 2H), 6.87 (br d, J=9.3 Hz, 1H), 6.77 (d, J=15.0 Hz, 1H), 4.23 (t, J=7.2 Hz, 2H), 4.11 (q, J=6.3 Hz, 2H), 3.78 (br s, 4H), 2.89-2.80 (m, 2H), 2.70-2.38 (m, 17H), 2.37-2.27 (m, 5H), 2.01-1.90 (m, 2H), 1.42 (t, J=6.9 Hz, 3H). LCMS: m/z 576.51 [M+H]⁺.

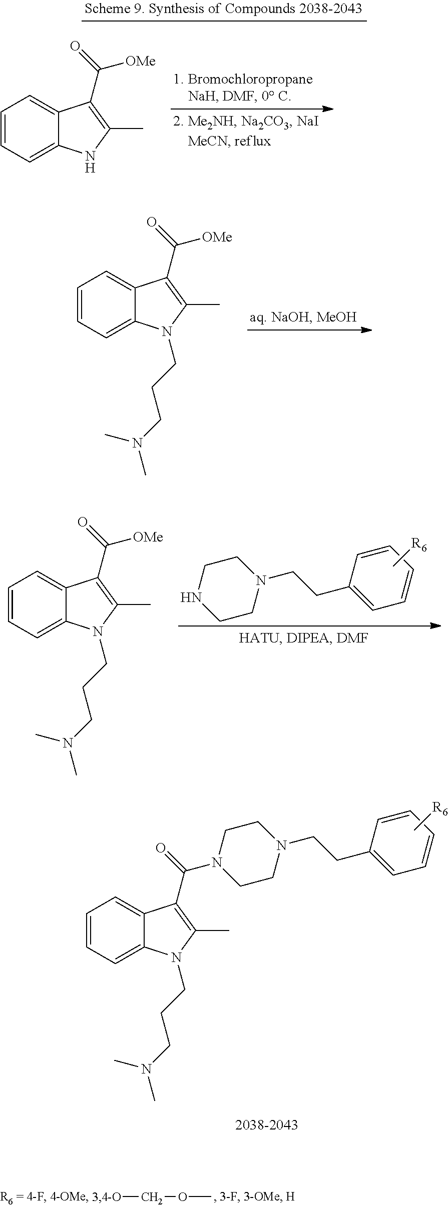

### Preparation of methyl 1-(3-chloropropyl)-2-methyl-1H-indole-3-carboxylate

NaH (253 mg, 10.57 mmol) was added portionwise to a stirred solution of methyl 2-methyl-1H-indole-3-carboxylate (1.0 g, 5.28 mmol) in DMF (10 mL) at 0° C. The mixture was allowed to warm to room temperature and stirred for 30 minutes. Bromochloropropane (2.6 mL, 80 mmol) was added dropwise at 0° C., after which the mixture was allowed to warm to room temperature and stirred for 3 hours. After complete consumption of the starting material, ice cold water was added to the reaction mixture, which was then extracted with ethyl acetate. The organic layer was washed with brine solution, dried over anhydrous Na₂SO₄ and concentrated under reduced pressure in order to afford a yellow liquid (1.3 g, 92%).

¹H NMR (400 MHz, CDCl₃): δ 8.17-8.09 (m, 1H), 7.39-7.32 (m, 1H), 7.29-7.20 (m, 2H), 4.36 (t, J=7.2 Hz, 2H), 3.92 (s, 3H), 3.53 (t, J=7.2 Hz, 2H), 2.81 (s, 3H), 2.22 (quintet, J=7.2 Hz, 2H). LCMS: m/z 266.46 [M+H]⁺.

### Preparation of methyl 1-(3-(dimethylamino)propyl)-2-methyl-1H-indole-3-carboxylate

To a stirred solution of methyl 1-(3-chloropropyl)-2-methyl-1H-indole-3-carboxylate (1.3 g, 4.89 mmol) in acetonitrile (20 mL) at room temperature was added sodium iodide (1.46 g, 9.7 mmol) and sodium carbonate (2.6 g, 24.4 mmol), followed by N,N-dimethylamine hydrochloride (1.57 g, 19.56 mmol). The reaction mixture was heated to 75° C. for 16 hours. After complete consumption of the starting material, the reaction mass was concentrated, diluted with water and extracted with ethyl acetate. The combined organic layers were washed with water and brine, dried over anhydrous Na₂SO₄ and concentrated under reduced pressure to afford the crude product. The crude compound was purified by flash column chromatography using 4-5% MeOH in DCM as an eluent to obtain a yellow liquid (1.2 g, 89%).

¹H NMR (400 MHz, CDCl₃): δ 8.14-8.08 (m, 1H), 7.40-7.33 (m, 1H), 7.29-7.18 (m, 2H), 4.22 (t, J=6.8 Hz, 2H), 3.93 (s, 3H), 2.79 (s, 3H), 2.28 (t, J=6.8 Hz, 2H), 2.24 (s, 6H), 1.93 (quintet, J=6.8 Hz, 2H). LCMS: m/z 275.47 [M+H]⁺.

### Preparation of 1-(3-(dimethylamino)propyl)-2-methyl-1H-indole-3-carboxylic acid

To a stirred solution of methyl 1-(3-(dimethylamino)propyl)-2-methyl-1H-indole-3-carboxylate (3.0 g, 10.9 mmol) in MeOH/H₂O (20 mL, 1:1), was added NaOH (4.37 g, 109 mmol) at 0° C. The mixture was stirred at 60° C. for 16 h. After complete consumption of the starting material, solvent was evaporated under vacuum and the residue was cooled to room temperature, acidified (to pH 1) with 1 N HCl, and then extracted with EtOAc. The organic layers were dried over anhydrous Na₂SO₄ and concentrated under reduced pressure to afford an off white solid (2.0 g, 70%).

¹H NMR (300 MHz, DMSO-d₆): δ 11.82 (br s, 1H), 7.99 (br d, J=7.5 Hz, 1H), 7.53 (br d, J=7.5 Hz), 7.30-7.11 (m, 2H), 4.21 (t, J=6.9 Hz, 2H), 2.77 (s, 3H), 2.87-2.80 (m, 2H), 2.24 (s, 6H), 1.94-1.80 (m, 2H). LCMS: m/z 261.44 [M+H]⁺.

### Preparation of Compound 2038, (1-(3-(dimethylamino)propyl)-2-methyl-1H-indol-3-yl)(4-(4-fluorophenethyl)piperazin-1-yl)methanone

To a stirred solution of 1-(3-(dimethylamino)propyl)-2-methyl-1H-indole-3-carboxylic acid (330 mg, 1.26 mmol) in DMF (5 mL), DIPEA (1.1 mL, 6.5 mmol) was added at room temperature. After 10 minutes stirring, HATU (965 mg, 2.53 mmol) was added and the mixture was stirred for a further 30 min at room temperature. 1-(4-fluorophenethyl)piperazine (312 mg, 1.50 mmol) was added at 0° C. and the reaction mixture was stirred at room temperature for 16 hours. After complete consumption of the starting material, the reaction mixture was poured into ice water and extracted with EtOAc. The organic layer was dried over anhydrous Na₂SO₄ and concentrated under reduced pressure to afford the crude product. The crude compound was purified by flash column using 3-5% MeOH in DCM as an eluent to obtain an off-white solid (50 mg, 10%).

¹H NMR (400 MHz, CD₃OD): δ 7.50-7.45 (m, 2H), 7.26-7.13 (m, 4H), 7.00 (t, J=8.8 Hz, 2H), 4.32 (t, J=7.2 Hz, 2H), 3.71 (br s, 4H), 2.74-2.53 (m, 16H), 2.52 (s, 3H), 2.24-2.12 (m, 2H). LCMS: m/z 451.50 [M+H]⁺.

Other analogues prepared by this method:

Compound 2039, (1-(3-(dimethylamino)propyl)-2-methyl-1H-indol-3-yl)(4-(4-methoxyphenethyl)piperazin-1-yl)methanone (31%).

¹H NMR (300 MHz, CD₃OD): δ 7.44 (br d, J=8.7 Hz, 2H), 7.22-7.10 (m, 4H), 6.86 (d, J=8.7 Hz, 2H), 4.24 (t, J=7.5 Hz, 2H), 3.75 (s, 3H), 3.68 (br s, 4H), 2.80-2.71 (m, 2H), 2.67-2.40 (m, 11H), 2.50 (s, 6H), 1.97 (quintet, J=7.2 Hz, 2H). LCMS: m/z 463.5 [M+H]⁺.

Compound 2040, (4-(2-(benzo[d][1,3]dioxol-5-yl)ethyl)piperazin-1-yl)(1-(3-(dimethylamino)propyl)-2-methyl-1H-indol-3-yl)methanone (11%).

¹H NMR (400 MHz, CD₃OD): δ 7.47-7.44 (m, 2H), 7.21 (br t, J=7.2 Hz, 1H), 7.15 (br t, J=7.2 Hz, 1H), 6.74-6.64 (m, 3H), 5.88 (s, 2H), 4.29 (t, J=7.6 Hz, 2H), 3.68 (br s, 4H), 2.91-2.85 (m, 2H), 2.77-2.72 (m, 2H), 2.69-2.48 (m, 15H), 2.14-2.06 (m, 2H). LCMS: m/z 477.47 [M+H]⁺.

Compound 2041, (1-(3-(dimethylamino)propyl)-5-methoxy-2-methyl-1H-indol-3-yl)(4-(3-fluorophenethyl)piperazin-1-yl)methanone (12%).

¹H NMR (300 MHz, CD₃OD): δ 7.48-7.43 (m, 2H), 7.32-7.11 (m, 3H), 7.04 (br d, J=8.1 Hz, 1H), 6.98 (br d, J=10.2 Hz, 1H), 6.91 (br t, J=8.7 Hz, 1H), 4.28 (t, J=7.2 Hz, 2H), 3.68 (br s, 4H), 2.89-2.77 (m, 4H), 2.71-2.47 (m, 15H), 2.14-2.03 (m, 2H). LCMS: m/z 451.50 [M+H]⁺.

Compound 2042, (1-(3-(dimethylamino)propyl)-2-methyl-1H-indol-3-yl)(4-(3-methoxyphenethyl)piperazin-1-yl)methanone (12%).

¹H NMR (400 MHz, CD₃OD): δ 7.48-7.44 (m, 2H), 7.30-7.13 (m, 3H), 6.82-6.73 (m, 3H), 4.30 (t, J=7.6 Hz, 2H), 3.77 (s, 3H), 3.70 (br s, 4H), 2.84-2.52 (m, 14H), 2.51 (s, 3H), 2.21-2.12 (m, 2H). LCMS: m/z 463.5 [M+H]⁺.

Compound 2043, (1-(3-(dimethylamino)propyl)-2-methyl-1H-indol-3-yl)(4-phenethylpiperazin-1-yl)methanone (11%).

¹H NMR (400 MHz, CD₃OD): δ 7.45 (br d, J=8.8 Hz, 2H), 7.30-7.12 (m, 7H), 4.27 (t, J=7.2 Hz, 2H), 3.69 (br s, 4H), 2.85-2.79 (m, 2H), 2.74-2.46 (m, 17H), 2.10-2.00 (m, 2H). LCMS: m/z 433.54 [M+H]⁺.

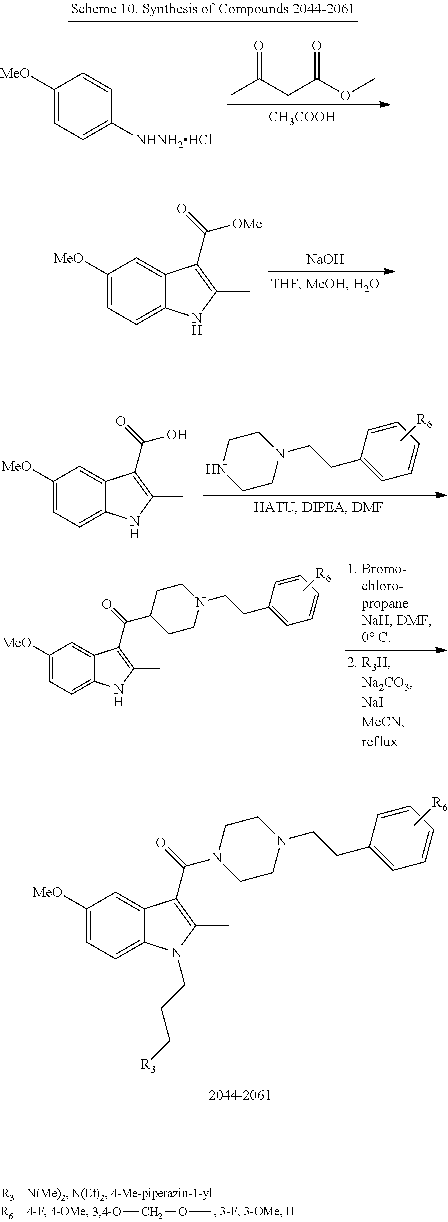

### Preparation of methyl 5-methoxy-2-methyl-1H-indole-3-carboxylate

Methyl acetoacetate (15.9 mL, 0.147 mol) was added to a stirred solution of (4-methoxyphenyl)hydrazine hydrochloride (20 g, 114.5 mmol) in glacial acetic acid (200 mL). The reaction mixture was heated to 110° C. for 2 hours, then cooled to stir at room temperature for a further 16 hours. After complete consumption of the starting material, acetic acid was evaporated under vacuum. Crushed ice was added to the residue and the resultant was basified (pH 14) with 1 N NaOH and extracted with DCM. The organic layer was concentrated under reduced pressure to afford the crude product. The crude compound was purified by flash column chromatography using 10% EtOAc in petroleum ether as an eluent to obtain a brown liquid (3.4 g, 12%).

¹H NMR (400 MHz, DMSO-d₆): δ 11.66 (br s, 1H), 7.42 (d, J=2.0 Hz, 1H), 7.24 (d, J=8.4 Hz, 1H), 6.77 (dd, J=8.4 Hz, 2.0 Hz, 1H), 3.82 (s, 3H), 3.78 (s, 3H), 2.61 (s, 3H). LCMS: m/z 220.2 [M+H]⁺.

### Preparation of 5-methoxy-2-methyl-1H-indole-3-carboxylic acid

Methyl 5-methoxy-2-methyl-1H-indole-3-carboxylate (3.4 g, 15.50 mmol) was dissolved in THF:H₂O:MeOH (60 mL, 1:1:1). NaOH (1 N, 20 mL) was added at room temperature. The reaction mixture was heated to 70° C. for 16 hours. After complete consumption of the starting material, solvent was evaporated under vacuum and the residue was acidified (pH 1) with 1N HCl and extracted with ethyl acetate. The organic layer was dried over anhydrous Na₂SO₄ and concentrated under reduced pressure to give an off white solid (1.68 g, 54%).

¹H NMR (400 MHz, DMSO-d₆): δ 11.78 (br s, 1H), 11.54 (br s, 1H), 7.43 (d, J=2.0 Hz, 1H), 7.22 (d, J=8.4 Hz, 1H), 6.74 (dd, J=8.4 Hz, 2.0 Hz, 1H), 3.76 (s, 3H), 2.61 (s, 3H). LCMS: m/z 206.42 [M+H]⁺.

### Preparation of (4-(4-fluorophenethyl)piperazin-1-yl)(5-methoxy-2-methyl-1H-indol-3-yl)methanone

To a stirred solution of 5-methoxy-2-methyl-1H-indole-3-carboxylic acid (550 mg, 2.68 mmol) in DMF (5 mL), DIPEA (1.1 mL, 6.3 mmol) was added. The mixture was stirred for 10 minutes, after which HATU (1.12 g, 2.95 mmol) was added and the mixture was stirred for a further 30 minutes at room temperature. The reaction mass was cooled to 0° C., 1-(4-fluorophenethyl)piperazine (652 mg, 2.68 mmol) was added and the reaction mixture was allowed to warm to room temperature and stirred for 16 hours. After complete consumption of the starting material, the reaction mixture was poured into ice water and extracted with EtOAc. The organic layer was dried over anhydrous Na₂SO₄ and concentrated under reduced pressure to afford the crude product. The crude compound was purified by flash column chromatography using 2-3% MeOH in DCM as an eluent to obtain an off white solid (840 mg, 80%). LCMS: m/z 394.1 [M+H]⁺.

Other analogues prepared by this method:
(5-methoxy-2-methyl-1H-indol-3-yl)(4-(4-methoxyphenethyl)piperazin-1-yl)methanone (91%).
(4-(2-(benzo[d][1,3]dioxol-5-yl)ethyl)piperazin-1-yl)(5-methoxy-2-methyl-1H-indol-3-yl)methanone (70%).
(4-(3-fluorophenethyl)piperazin-1-yl)(5-methoxy-2-methyl-1H-indol-3-yl)methanone (43%)
(5-methoxy-2-methyl-1H-indol-3-yl)(4-(3-methoxyphenethyl)piperazin-1-yl)methanone (57%)
(5-methoxy-2-methyl-1H-indol-3-yl)(4-phenethylpiperazin-1-yl)methanone (88%)

### Preparation of (1-(3-chloropropyl)-5-methoxy-2-methyl-1H-indol-3-yl)(4-(4-fluorophenethyl)piperazin-1-yl)methanone

NaH (263 mg, 6.57 mmol) was added portionwise to a stirred solution of (4-(4-fluorophenethyl)piperazin-1-yl)(5-methoxy-2-methyl-1H-indol-3-yl)methanone (1.3 g, 3.28 mmol) in DMF (13 mL) at 0° C. The mixture was allowed to warm to room temperature and stirred for 30 minutes. To this, bromochloropropane (1.64 mL, 16.4 mmol) was added dropwise at 0° C. The mixture was allowed to warm to room temperature and was stirred for 3 hours. After complete consumption of the starting material, ice cold water was added to the reaction mixture and the resultant was extracted with ethyl acetate. The organic layer was washed with brine solution, dried over anhydrous Na₂SO₄ and concentrated under reduced pressure to afford the crude product. The crude compound was purified by flash column chromatography using EtOAc as an eluent to afford a brown gummy liquid (470 mg, 31%).

¹H NMR (400 MHz, DMSO-d₆): δ 7.39 (d, J=8.8 Hz, 1H), 7.35 (dd, J=8.4 Hz, 5.4 Hz, 2H), 7.00 (t, J=8.4 Hz, 2H), 6.86 (d, J=2.4 Hz, 1H), 6.78 (dd, J=8.8 Hz, 2.0 Hz, 1H), 4.22 (t, J=7.2 Hz, 2H), 3.76 (s, 3H), 3.62 (t, J=7.2 Hz, 2H), 3.59-3.37 (m, 4H), 2.79-2.68 (m, 2H), 2.59-2.32 (m, 9H), 2.18-2.07 (m, 2H). LCMS: m/z 472.14 [M+H]⁺.

Other analogues prepared by this method:
(1-(3-chloropropyl)-5-methoxy-2-methyl-1H-indol-3-yl)(4-(4-methoxyphenethyl)piperazin-1-yl)methanone (20%)
(4-(2-(benzo[d][1,3]dioxol-5-yl)ethyl)piperazin-1-yl)(1-(3-chloropropyl)-5-methoxy-2-methyl-1H-indol-3-yl)methanone (47%)
(1-(3-chloropropyl)-5-methoxy-2-methyl-1H-indol-3-yl)(4-(3-fluorophenethyl)piperazin-1-yl)methanone (70%)
(1-(3-chloropropyl)-5-methoxy-2-methyl-1H-indol-3-yl)(4-(3-methoxyphenethyl)piperazin-1-yl)methanone (84%)
(1-(3-chloropropyl)-5-methoxy-2-methyl-1H-indol-3-yl)(4-phenethylpiperazin-1-yl)methanone (69%)

### Preparation of Compound 2044, (1-(3-(dimethylamino)propyl)-5-methoxy-2-methyl-1H-indol-3-yl)(4-(4-fluorophenethyl)piperazin-1-yl)methanone

To a stirred solution of (1-(3-chloropropyl)-5-methoxy-2-methyl-1H-indol-3-yl)(4-(4-fluorophenethyl)piperazin-1-yl)methanone (250 mg, 0.52 mmol) in acetonitrile (10 mL) at room temperature, sodium iodide (159 mg, 1.05 mmol) and sodium carbonate (281 mg, 2.64 mmol) were added, followed by N,N-dimethylamine hydrochloride (171 mg, 2.11 mmol). The reaction mixture was heated to 70° C. for 16 hours. After complete consumption of the starting material, the reaction mixture was cooled to room temperature, diluted with EtOAc (60 mL), washed with water and brine solution, dried over anhydrous Na₂SO₄ and concentrated under reduced pressure to afford the crude product. The crude compound was purified by prep-TLC using 5% MeOH-DCM as an eluent to afford the target compound as a light brown liquid (48 mg, 19%).

¹H NMR (300 MHz, DMSO-d₆): δ 7.37 (d, J=8.7 Hz, 1H), 7.26 (dd, J=8.4 Hz, 5.7 Hz, 2H), 7.08 (t, J=9.0 Hz, 2H), 6.86 (d, J=2.1 Hz, 1H), 6.77 (dd, J=9.0 Hz, 2.4 Hz, 1H), 4.13 (t, J=7.2 Hz, 2H), 3.76 (s, 3H), 3.60-3.36 (m, 4H), 2.77-2.69 (m, 2H), 2.48-2.38 (m, 9H), 2.23-2.21 (m, 8H), 1.84-1.72 (m, 2H). LCMS: m/z 481.56 [M+H]⁺.

Other analogues prepared by this method:

Compound 2045, (1-(3-(dimethylamino)propyl)-5-methoxy-2-methyl-1H-indol-3-yl)(4-(4-methoxyphenethyl)piperazin-1-yl)methanone (6%).

¹H NMR (400 MHz, DMSO-d₆): δ 7.38 (d, J=8.8 Hz, 1H), 7.13 (d, J=8.1 Hz, 2H), 6.87 (d, J=2.4 Hz, 1H), 6.83 (d, J=8.1 Hz, 2H), 6.78 (dd, J=8.8 Hz, 2.4 HZ, 1H), 4.14 (t, J=7.6 Hz, 2H), 3.76 (s, 3H), 3.71 (s, 3H), 3.58-3.38 (m, 4H), 2.69-2.65 (m, 2H), 2.52-2.22 (m, 17H), 1.91-1.83 (m, 2H). LCMS: m/z 493.31 [M+H]⁺.

Compound 2046, (4-(2-(benzo[d][1,3]dioxol-5-yl)ethyl)piperazin-1-yl)(1-(3-(dimethylamino)propyl)-5-methoxy-2-methyl-1H-indol-3-yl)methanone (13%).

¹H NMR (300 MHz, DMSO-d₆): δ 7.37 (d, J=8.7 Hz, 1H), 6.86 (d, J=2.4 HZ, 1H), 6.82-6.74 (m, 3H), 6.67 (br d, J=8.1 Hz, 1H), 5.95 (s, 2H), 4.13 (t, J=7.2 Hz, 2H), 3.75 (s, 3H), 3.59-3.37 (m, 4H), 2.70-2.62 (m, 2H), 2.52-2.33 (m, 9H), 2.23-2.09 (m, 8H), 1.83-1.72 (m, 2H). LCMS: m/z 507.5 [M+H]⁺.

Compound 2047, (1-(3-(dimethylamino)propyl)-5-methoxy-2-methyl-1H-indol-3-yl)(4-(3-fluorophenethyl)piperazin-1-yl)methanone (8%).

¹H NMR (300 MHz, DMSO-d₆): δ 7.37 (d, J=9.0 Hz, 1H), 7.30 (td, J=8.1 Hz, 6.3 Hz, 1H), 7.12-7.04 (m, 2H), 6.99 (td, J=8.7 Hz, 2.7 Hz, 1H), 6.86 (d, J=2.4 Hz, 1H), 6.77 (dd, J=8.7 Hz, 2.7 Hz, 1H), 4.13 (t, J=6.9 Hz, 2H), 3.76 (s, 3H), 3.59-3.37 (m, 4H), 2.80-2.71 (m, 2H), 2.60-2.39 (m, 9H), 2.23-2.10 (m, 8H), 1.84-1.73 (m, 2H). LCMS: m/z 481.5 [M+H]⁺.

Compound 2048, (1-(3-(dimethylamino)propyl)-5-methoxy-2-methyl-1H-indol-3-yl)(4-(3-methoxyphenethyl)piperazin-1-yl)methanone (10%).

¹H NMR (400 MHz, DMSO-d₆): δ 7.37 (d, J=8.7 Hz, 1H), 7.17 (t, J=8.0 Hz, 1H), 6.86 (d, J=2.4 Hz, 1H), 6.83-6.72 (m, 4H), 4.13 (t, J=6.8 Hz, 2H), 3.75 (s, 3H), 3.72 (s, 3H), 3.54-3.40 (m, 4H), 2.74-2.66 (m, 2H), 2.57-2.38 (m, 9H), 2.23-2.10 (m, 8H), 1.84-1.76 (m, 2H). LCMS: m/z 493.31 [M+H]⁺.

Compound 2049, (1-(3-(dimethylamino)propyl)-5-methoxy-2-methyl-1H-indol-3-yl)(4-phenethylpiperazin-1-yl)methanone (18%).

¹H NMR (400 MHz, DMSO-d₆): δ 7.37 (d, J=8.4 Hz, 1H), 7.29-7.21 (m, 4H), 7.17 (t, J=6.8 Hz, 1H), 6.86 (d, J=2.4 Hz, 1H), 6.77 (dd, J=8.8 Hz, 2.4 Hz, 1H), 4.13 (t, J=6.8 Hz, 2H), 3.76 (s, 3H), 3.58-3.37 (m, 4H), 2.76-2.72 (m, 2H), 2.56-2.43 (m, 6H), 2.42 (s, 3H), 2.31-2.13 (m, 8H), 1.80 (quintet, J=6.8 Hz, 2H). LCMS: m/z 463.3 [M+H]⁺.

Compound 2050, (1-(3-(diethylamino)propyl)-5-methoxy-2-methyl-1H-indol-3-yl)(4-(4-fluorophenethyl)piperazin-1-yl)methanone (10%).

¹H NMR (300 MHz, DMSO-d₆): δ 7.38 (d, J=9.0 Hz, 1H), 7.26 (dd, J=8.4 Hz, 6.0 Hz, 2H), 7.08 (t, J=9.0 Hz, 2H), 6.86 (d, J=2.1 Hz, 1H), 6.77 (dd, J=8.7 Hz, 2.1 Hz, 1H), 4.13 (t, J=6.9 Hz, 2H), 3.76 (s, 3H), 3.58-3.36 (m, 4H), 2.78-2.69 (m, 2H), 2.58-2.28 (m, 15H), 1.84-1.70 (m, 2H), 0.93 (br s, 6H). LCMS: m/z 509.5 [M+H]⁺.

Compound 2051, (1-(3-(diethylamino)propyl)-5-methoxy-2-methyl-1H-indol-3-yl)(4-(4-methoxyphenethyl)piperazin-1-yl)methanone (20%).

¹H NMR (400 MHz, DMSO-d₆): δ 7.40 (d, J=8.4 Hz, 1H), 7.13 (d, J=8.4 Hz, 2H), 6.87 (d, J=2.0 Hz, 1H), 6.83 (d, J=8.8 Hz, 2H), 6.78 (dd, J=8.4 Hz, 2.0 Hz, 1H), 4.15 (br s, 2H), 3.76 (s, 3H), 3.72 (s, 3H), 3.55-3.38 (m, 4H), 2.70-2.64 (m, 2H), 2.58-2.32 (m, 15H), 1.84 (br s, 2H), 0.98 (br s, 6H). LCMS: m/z 521.3 [M+H]⁺.

Compound 2052, (4-(2-(benzo[d][1,3]dioxol-5-yl)ethyl)piperazin-1-yl)(1-(3-(diethylamino)propyl)-5-methoxy-2-methyl-1H-indol-3-yl)methanone (13%).

¹H NMR (300 MHz, DMSO-d₆): δ 7.40 (d, J=9.3 Hz, 1H), 6.88 (d, J=2.7 Hz, 1H), 6.86-6.75 (m, 3H), 6.69 (br d, J=8.1 Hz, 1H), 5.97 (s, 2H), 4.16 (t, J=6.9 Hz, 2H), 3.75 (s, 3H), 3.62-3.37 (m, 4H), 2.71-2.63 (m, 2H), 2.61-2.30 (m, 15H), 1.87-1.71 (m, 2H), 0.97 (t, J=7.2 Hz, 6H). LCMS: m/z 535.5 [M+H]⁺.

Compound 2053, (1-(3-(diethylamino)propyl)-5-methoxy-2-methyl-1H-indol-3-yl)(4-(3-fluorophenethyl)piperazin-1-yl)methanone (35%).

¹H NMR (300 MHz, DMSO-d₆): δ 7.40 (br d, 8.7 Hz, 1H), 7.30 (td, J=8.1 Hz, 6.3 Hz, 1H), 7.12-7.00 (m, 2H), 6.87 (d, J=2.1 Hz, 1H), 6.79 (dd, J=8.7 Hz, 2.1 Hz, 1H), 4.15 (t, J=6.9 Hz, 2H), 3.76 (s, 3H), 3.59-3.37 (m, 4H), 2.81-2.70 (m, 2H), 2.61-2.30 (m, 15H), 1.92-1.69 (m, 2H), 0.97 (br s, 6H). LCMS: m/z 509.5 [M+H]⁺.

Compound 2054, (1-(3-(diethylamino)propyl)-5-methoxy-2-methyl-1H-indol-3-yl)(4-(3-methoxyphenethyl)piperazin-1-yl)methanone (15%).

¹H NMR (400 MHz, DMSO-d₆): δ 7.38 (d, J=8.8 Hz, 1H), 7.18 (t, J=7.2 Hz, 1H), 6.86 (d, J=2.4 Hz, 1H), 6.81-6.72 (m, 4H), 4.13 (t, J=7.2 Hz, 2H), 3.76 (s, 3H), 3.72 (s, 3H), 3.59-3.38 (m, 4H), 2.73-2.66 (m, 2H), 2.56-2.39 (m, 15H), 1.83-1.74 (m, 2H), 0.94 (t, J=6.8 Hz, 6H). LCMS: m/z 521.4 [M+H]⁺.

Compound 2055, (1-(3-(diethylamino)propyl)-5-methoxy-2-methyl-1H-indol-3-yl)(4-phenethylpiperazin-1-yl)methanone (14%).

¹H NMR (400 MHz, DMSO-d₆): δ 7.37 (d, J=8.8 Hz, 1H), 7.29-7.20 (m, 4H), 7.17, (t, J=7.2 Hz, 1H), 6.86 (d, J=2.4 Hz, 1H), 6.77 (dd, J=8.8 Hz, 2.4 Hz, 1H), 4.13 (t, J=7.2 Hz, 2H), 3.75 (s, 3H), 3.58-3.39 (m, 4H), 2.77-2.71 (m, 2H), 2.56-2.33 (m, 15H), 1.82-1.73 (m, 2H), 0.94 (t, J=7.2 Hz, 6H). LCMS: m/z 491.4 [M+H]⁺.

Compound 2056, (4-(4-fluorophenethyl)piperazin-1-yl)(5-methoxy-2-methyl-1-(3-(4-methylpiperazin-1-yl)propyl)-1H-indol-3-yl)methanone (12%).

¹H NMR (400 MHz, CD₃OD): δ 7.38 (d, J=8.8 Hz, 1H), 7.26 (dd, J=8.4 Hz, 6.0 Hz, 2H), 7.08 (t, J=9.6 Hz, 2H), 6.86 (d, J=2.4 Hz, 1H), 6.76 (dd, J=8.8 Hz, 2.4 Hz, 1H), 4.13 (t, J=6.8 Hz, 2H), 3.75 (s, 3H), 3.58-3.36 (m, 4H), 2.78-2.69 (m, 2H), 2.54-2.23 (m, 17H), 2.21-2.12 (m, 5H), 1.87-1.78 (m, 2H). LCMS: m/z 536.54 [M+H]⁺.

Compound 2057, (5-methoxy-2-methyl-1-(3-(4-methylpiperazin-1-yl)propyl)-1H-indol-3-yl)(4-(4-methoxyphenethyl)piperazin-1-yl)methanone (30%).

¹H NMR (400 MHz, CD₃OD): δ 7.38 (d, J=8.8 Hz, 1H), 7.13 (d, J=8.4 Hz, 2H), 6.86 (d, J=2.4 Hz, 1H), 6.83 (d, J=8.4 Hz, 2H), 6.76 (dd, J=8.8 Hz, 2.4 Hz, 1H), 4.13 (t, J=6.8 Hz, 2H), 3.76 (s, 3H), 3.71 (s, 3H), 3.57-3.38 (m, 4H), 2.69-2.65 (m, 2H), 2.50-2.23 (m, 17H), 2.20 (br t, J=6.4 Hz, 2H), 2.15 (s, 3H), 1.82 (quintet, J=6.4 Hz, 2H). LCMS: m/z 548.3 [M+H]⁺.

Compound 2058, (4-(2-(benzo[d][1,3]dioxol-5-yl)ethyl)piperazin-1-yl)(5-methoxy-2-methyl-1-(3-(4-methylpiperazin-1-yl)propyl)-1H-indol-3-yl)methanone (14%).

¹H NMR (300 MHz, DMSO-d₆): δ 7.38 (d, J=8.7 Hz, 1H), 6.86 (d, J=2.4 Hz, 1H), 6.84-6.72 (m, 3H), 6.67 (br d, J=7.8 Hz, 1H), 5.95 (s, 2H), 4.13 (t, J=6.9 Hz, 2H), 3.75 (s, 3H), 3.58-3.37 (m, 4H), 2.70-2.61 (m, 2H), 2.58-2.11 (m, 22H), 1.87-1.75 (m, 2H). LCMS: m/z 562.4 [M+H]⁺.

Compound 2059, (4-(3-fluorophenethyl)piperazin-1-yl)(5-methoxy-2-methyl-1-(3-(4-methylpiperazin-1-yl)propyl)-1H-indol-3-yl)methanone (31%).

¹H NMR (300 MHz, DMSO-d₆): δ 7.38 (d, J=8.7 Hz, 1H), 7.31 (td, J=8.1 Hz, 6.0 Hz, 1H), 7.12-7.05 (m, 2H), 6.86 (d, J=2.1 Hz, 1H), 6.76 (dd, J=9.0 Hz, 2.4 Hz, 1H), 4.13 (t, J=6.9 Hz, 2H), 3.75 (s, 3H), 3.57-3.38 (m, 4H), 2.81-2.71 (m, 2H), 2.60-2.23 (m, 17H), 2.22-2.14 (m, 5H), 1.88-1.77 (m, 2H). LCMS: m/z 536.5 [M+H]⁺.

Compound 2060, (5-methoxy-2-methyl-1-(3-(4-methylpiperazin-1-yl)propyl)-1H-indol-3-yl)(4-(3-methoxyphenethyl)piperazin-1-yl)methanone (8%)

¹H NMR (400 MHz, DMSO-d₆): δ 7.38 (d, J=8.8 Hz, 1H), 7.18 (t, J=7.6 Hz, 1H), 6.86 (d, J=1.6 Hz, 1H), 6.80-6.72 (m, 4H), 4.13 (t, J=6.8 Hz, 2H), 3.75 (s, 3H), 3.57-3.39 (m, 4H), 2.73-2.67 (m, 2H), 2.57-2.23 (m, 17H), 2.22-2.14 (m, 5H), 1.86-1.77 (m, 2H). LCMS: m/z 548.34 [M+H]⁺.

Compound 2061, (5-methoxy-2-methyl-1-(3-(4-methylpiperazin-1-yl)propyl)-1H-indol-3-yl)(4-phenethylpiperazin-1-yl)methanone (10%).

¹H NMR (400 MHz, DMSO-d₆): δ 7.38 (d, J=9.2 Hz, 1H), 7.29-7.20 (m, 4H), 7.17 (t, J=6.8 Hz, 1H), 6.86 (d, J=2.4 Hz, 1H), 6.76 (dd, J=8.8 Hz, 2.4 Hz, 1H), 4.13 (t, J=6.8 Hz, 2H), 3.75 (s, 3H), 3.58-3.38 (m, 4H), 2.76-2.72 (m, 2H), 2.56-2.23 (m, 17H), 2.22-2.16 (m, 5H), 1.82 (quintet, J=6.8 Hz, 2H). LCMS: m/z 518.38[M+H]⁺.

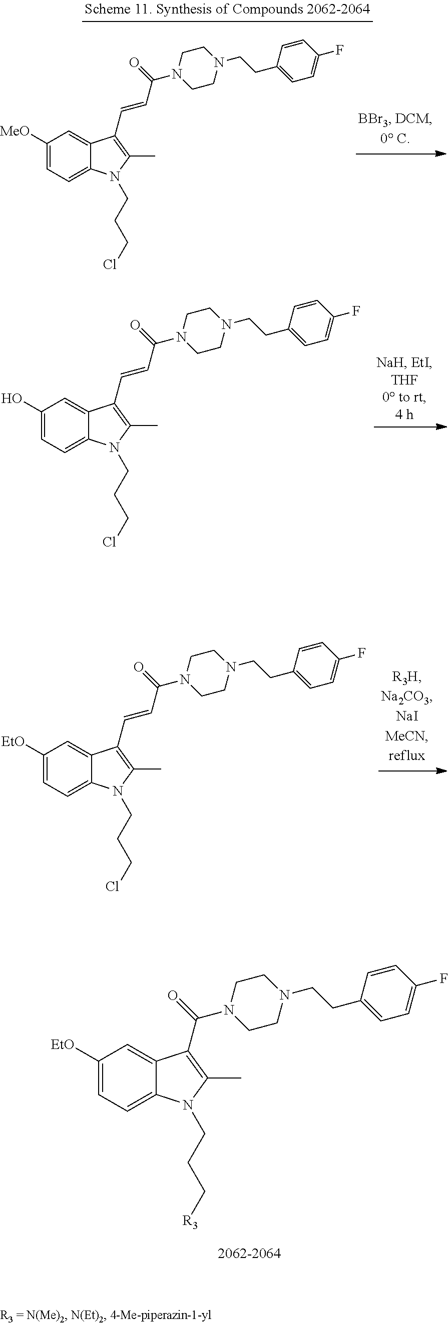

### Preparation of (1-(3-chloropropyl)-5-hydroxy-2-methyl-1H-indol-3-yl)(4-(4-fluorophenethyl)piperazin-1-yl)methanone

To a stirred solution of (1-(3-chloropropyl)-5-methoxy-2-methyl-1H-indol-3-yl)(4-(4-fluorophenethyl)piperazin-1-yl)methanone (720 mg, 1.52 mmol) in dry DCM (20 mL) was added boron tribromide (0.38 mL, 3.82 mmol) at 0° C. The mixture was allowed to warm to room temperature and was stirred for 4 hours. After complete consumption of the starting material, the reaction mixture was poured into saturated NaHCO₃ solution. The resultant was adjusted to pH 7 and extracted with DCM. The organic layer was washed with brine solution, dried over anhydrous Na₂SO₄ and concentrated under reduced pressure to afford a brown solid (560 mg, 80%).

¹H NMR (400 MHz, DMSO-d₆): δ 8.84 (br s, 1H), 7.33-7.25 (m, 3H), 7.09 (t, J=9.2 Hz, 2H), 6.78 (d, J=2.4 Hz, 1H), 6.64 (dd, J=8.8 Hz, 2.4 Hz, 1H), 4.19 (t, J=6.8 Hz, 2H), 3.65 (t, J=6.8 Hz, 2H), 3.58-3.34 (m, 4H), 2.79-2.70 (m, 2H), 2.58-2.42 (m, 6H), 2.40 (s, 3H), 2.15-2.04 (m, 2H). LCMS: m/z 458.23[M+H]⁺.

### Preparation of (1-(3-chloropropyl)-5-ethoxy-2-methyl-1H-indol-3-yl)(4-(4-fluorophenethyl)piperazin-1-yl)methanone

NaH (73 mg, 1.8 mmol) was added portionwise to a stirred solution of (1-(3-chloropropyl)-5-hydroxy-2-methyl-1H-indol-3-yl)(4-(4-fluorophenethyl)piperazin-1-yl)methanone (560 mg, 1.22 mmol) in DMF (15 mL) at 0° C. The mixture was allowed to warm to room temperature and was stirred for 2 hours. To this, ethyl iodide (0.19 mL, 2.44 mmol) was added dropwise at 0° C. The mixture was allowed to warm to room temperature and was stirred for 2 hours. After complete consumption of the starting material, ice cold water was added and the reaction mixture was extracted with ethyl acetate. The organic layer was washed with brine solution, dried over anhydrous Na₂SO₄ and concentrated under reduced pressure to afford the crude product. The crude compound was purified by flash column chromatography using 5% MeOH in DCM as an eluent to afford a brown gummy liquid (450 mg, 76%).

¹H NMR (400 MHz, CDCl₃): δ 7.25 (d, J=8.8 Hz, 1H), 7.16 (dd, J=8.8 Hz, 6.0 Hz, 2H), 7.00-6.89 (m, 3H), 6.82 (dd, J=8.8 Hz, 2.4 Hz, 1H), 4.19 (t, J=6.8 Hz, 2H), 4.08 (q, J=6.8 Hz, 2H), 3.64 (br s, 4H), 3.56 (t, J=6.8 Hz, 2H), 3.58-3.34 (m, 4H), 2.80-2.69 (m, 2H), 2.62-2.40 (m, 9H), 2.21 (quintet, J=6.8 Hz, 2H). LCMS: m/z 487.0[M+H]⁺.

### Preparation of Compound 2062, (1-(3-(dimethylamino)propyl)-5-ethoxy-2-methyl-1H-indol-3-yl)(4-(4-fluorophenethyl)piperazin-1-yl)methanone

To a stirred solution of (1-(3-chloropropyl)-5-ethoxy-2-methyl-1H-indol-3-yl)(4-(4-fluorophenethyl)piperazin-1-yl)methanone (150 mg, 0.31 mmol) in acetonitrile (10 mL) at room temperature, sodium iodide (92 mg, 0.61 mmol) and sodium carbonate (164 mg, 1.54 mmol) were added, followed by dimethylamine hydrochloride (100 mg, 1.2 mmol). The reaction mixture was heated to 70° C. for 16 hours. After complete consumption of the starting material, the reaction mixture was cooled to room temperature, diluted with EtOAc (60 mL), washed with water and brine solution, dried over anhydrous Na₂SO₄ and concentrated under reduced pressure to afford the crude product. The crude compound was purified by prep-TLC using 5% MeOH-DCM as an eluent to afford the target compound as a light brown liquid (17 mg, 4%).

¹H NMR (300 MHz, DMSO-d₆): δ 7.36 (d, J=9.3 Hz, 1H), 7.26 (dd, J=8.4 Hz, 5.7 Hz, 2H), 7.08 (t, J=8.7 Hz, 2H), 6.84 (d, J=2.4 Hz, 1H), 6.76 (dd, J=8.7 Hz, 2.1 Hz, 1H), 4.13 (t, J=7.2 Hz, 2H), 4.00 (q, J=6.9 Hz, 2H), 3.47 (br s, 4H), 2.78-2.69 (m, 2H), 2.58-2.37 (m, 9H), 2.30-2.13 (m, 8H), 1.88-1.75 (m, 2H), 1.33 (t, J=6.9 Hz, 3H). LCMS: m/z 495.3 [M+H]⁺.

Other analogues prepared by this method:

Compound 2063, (1-(3-(diethylamino)propyl)-5-ethoxy-2-methyl-1H-indol-3-yl)(4-(4-fluorophenethyl)piperazin-1-yl)methanone (16%).

¹H NMR (300 MHz, DMSO-d₆): δ 7.39 (br d, J=8.7 Hz, 1H), 7.26 (dd, J=8.7 Hz, 5.7 Hz, 2H), 7.08 (t, J=8.7 Hz, 2H), 6.84 (d, J=2.1 Hz, 1H), 6.77 (br d, J=8.7 Hz, 1H), 4.14 (br s, 2H), 4.01 (q, J=6.9 Hz, 2H), 3.48 (br s, 4H), 2.78-2.68 (m, 2H), 2.58-2.30 (m, 15H), 2.00-1.70 (m, 2H), 1.33 (t, J=6.9 Hz, 3H), 0.99 (br s, 6H). LCMS: m/z 523.49 [M+H]⁺.

Compound 2064, (5-ethoxy-2-methyl-1-(3-(4-methylpiperazin-1-yl)propyl)-1H-indol-3-yl)(4-(4-fluorophenethyl)piperazin-1-yl)methanone (11%).

¹H NMR (300 MHz, DMSO-d₆): δ 7.39 (d, J=8.7 Hz, 1H), 7.26 (dd, J=8.4 Hz, 5.7 Hz, 2H), 7.08 (t, J=9.0 Hz, 2H), 6.84 (d, J=2.4 Hz, 1H), 6.75 (dd, J=9.0 Hz, 2.4 Hz, 1H), 4.12 (t, J=6.6 Hz, 2H), 4.00 (q, J=6.9 Hz, 2H), 3.47 (br s, 4H), 2.78-2.68 (m, 2H), 2.59-2.24 (m, 17H), 2.22-2.14 (m, 5H), 1.88-1.73 (m, 2H), 1.33 (t, J=6.9 Hz, 3H). LCMS: m/z 550.3 [M+H]⁺.

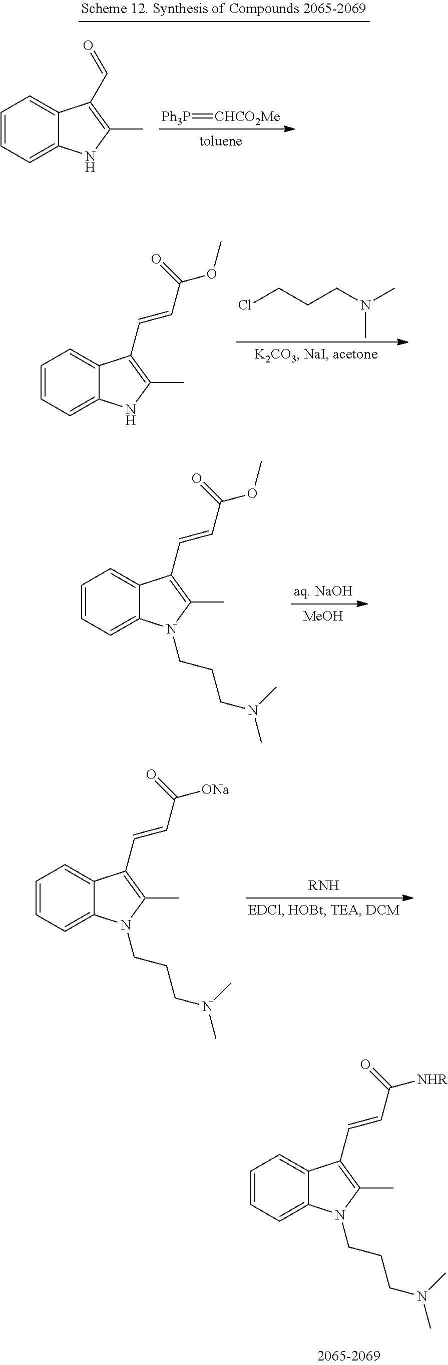

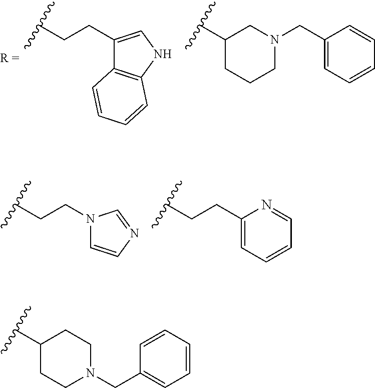 [SMILES: *CCc1c[nH]c2ccccc12 ; *C1CCCN(Cc2ccccc2)C1 ; *CCn1ccnc1 ; *CCc1ccccn1 ; *C1CCN(Cc2ccccc2)CC1]

### Preparation of methyl (E)-3-(2-methyl-1H-indol-3-yl)acrylate

A mixture of 2-methyl-1H-indole-3-carbaldehyde (1.00 g, 6.28 mmol) and methyl 2-(triphenyl-λ⁵-phosphanylidene)acetate (2.10 g, 6.28 mmol) in toluene (30 mL) was heated under reflux overnight, then concentrated. The residue was purified by silica gel column chromatography (Petroleum ether/EtOAc=5/0˜1, v/v) to give a white solid (1.10 g, 81%).

¹H NMR (400 MHz, CDCl₃): δ 8.28 (br s, 1H), 7.98 (d, J=16.0 Hz, 1H), 7.89-7.87 (m, 1H), 7.36-7.33 (m, 1H), 7.25-7.23 (m, 2H), 6.46 (d, J=16.0 Hz, 1H), 3.84 (s, 3H), 2.59 (s, 3H).

### Preparation of methyl (E)-3-(1-(3-(dimethylamino)propyl)-2-methyl-1H-indol-3-yl)acrylate

A mixture of methyl (E)-3-(2-methyl-1H-indol-3-yl)acrylate (100 mg, 0.46 mmol), 3-(N,N-dimethylamino)propyl chloride (109 mg, 0.69 mmol), K₂CO₃ (318 mg, 2.30 mmol) and NaI (76 mg, 0.51 mmol) in acetone (20 mL) was heated at reflux for 3 days, then it was filtered and concentrated. The crude product was dissolved in ethyl acetate, washed with brine and dried over Na₂SO₄. Removal of the solvent afforded the target compound (70 mg, 46%).

¹H NMR (400 MHz, CDCl₃): δ 8.00 (d, J=15.6 Hz, 1H), 7.90-7.88 (m, 1H), 7.40-7.38 (m, 1H), 7.27-7.21 (m, 2H), 6.43 (d, J=15.6 Hz, 1H), 4.20 (t, J=7.2 Hz, 2H), 3.82 (s, 3H), 2.58 (s, 3H), 2.29 (t, J=6.8 Hz, 2H), 2.24 (s, 6H), 1.96-1.89 (m, 2H).

### Preparation of sodium (E)-3-(1-(3-(dimethylamino)propyl)-2-methyl-1H-indol-3-yl)acrylate

A solution of NaOH (1.86 g, 46.60 mmol) in water (50 mL), was added to a stirring mixture of methyl (E)-3-(1-(3-(dimethylamino)propyl)-2-methyl-1H-indol-3-yl)acrylate (7.00 g, 23.30 mmol) in methanol (100 mL). After stirring at room temperature for 2 days, methanol was removed under reduced pressure. The aqueous phase was washed with EtOAc and adjusted to pH 7. The resulting precipitate was filtered to give a white solid (5.0 g, 55.9%).

¹H NMR (400 MHz, DMSO-d₆): δ 7.81 (br d, J=7.2 Hz, 1H), 7.81 (d, J=15.6 Hz, 1H), 7.53 (br d, J=7.2 Hz, 1H), 7.23-7.15 (m, 2H), 6.30 (d, J=15.6 Hz, 1H), 4.21 (t, J=7.2 Hz, 2H), 2.55 (s, 3H), 2.21 (t, J=6.8 Hz, 2H), 2.14 (s, 6H), 1.85-1.78 (m, 2H).

### Preparation of Compound 2065, (E)-N-(2-(1H-indol-3-yl)ethyl)-3-(1-(3-(dimethylamino)propyl)-2-methyl-1H-indol-3-yl)acrylamide

A mixture of sodium (E)-3-(1-(3-(dimethylamino)propyl)-2-methyl-1H-indol-3-yl)acrylate (200 mg, 0.65 mmol), 2-(1H-indol-3-yl)ethan-1-amine (104 mg, 0.65 mmol), EDCl (248 mg, 1.35 mmol), HOBt (88 mg, 0.65 mmol) and TEA (136 mg, 1.35 mmol) in DCM (15 mL) was stirred at room temperature overnight. The reaction mixture was washed with saturated Na₂CO₃, dried over Na₂SO₄ and concentrated. The crude product was purified by preparative TLC to give a solid (75 mg, 14%).

¹H NMR (400 MHz, CDCl₃): δ 8.17 (br s, 1H), 7.96 (d, J=15.6 Hz, 1H), 7.81 (d, J=7.6 Hz, 1H), 7.70 (d, J=8.0 Hz, 1H), 7.42 (d, J=8.0 Hz, 1H), 7.37 (d, J=8.0 Hz, 1H), 7.26-7.15 (m, 4H), 7.11 (d, J=2.4 Hz, 1H), 6.32 (d, J=15.6 Hz, 1H), 5.68 (t, J=6.0 Hz, 1H), 4.21 (t, J=7.2 Hz, 2H), 3.80 (q, J=6.4 Hz, 2H), 3.10 (t, J=6.4 Hz, 2H), 2.56 (s, 3H), 2.38 (t, J=6.8 Hz, 2H), 2.31 (s, 6H), 2.02-1.95 (m, 2H). LCMS: m/z 429.1 [M+H]⁺.

### Preparation of Compound 2066, (E)-N-(1-benzylpiperidin-3-yl)-3-(1-(3-(dimethylamino)propyl)-2-methyl-1H-indol-3-yl)acrylamide

A mixture of sodium (E)-3-(1-(3-(dimethylamino)propyl)-2-methyl-1H-indol-3-yl)acrylate (200 mg, 0.65 mmol), 1-benzylpiperidin-3-amine (104 mg, 0.65 mmol), EDCl (248 mg, 1.35 mmol), HOBt (88 mg, 0.65 mmol) and TEA (136 mg, 1.35 mmol) in DCM (15 mL) was stirred at room temperature overnight. The reaction mixture was washed with saturated Na₂CO₃, dried over Na₂SO₄ and concentrated. The crude product was purified by preparative TLC to give a solid (63 mg, 21%).

¹H NMR (400 MHz, CDCl₃): δ 7.96-7.92 (m, 2H), 7.40-7.25 (m, 8H), 7.46 (d, J=15.6 Hz, 1H), 4.34 (br s, 1H), 4.23 (t, J=7.2 Hz, 2H), 3.63 (br s, 2H), 2.76-2.60 (m, 3H), 2.58 (s, 3H), 2.41-2.32 (m, 9H), 2.04-1.97 (m, 2H), 1.90-1.62 (m, 4H). LCMS: m/z 459.1 [M+H]⁺.

### Preparation of Compound 2067, (E)-N-(2-(1H-imidazol-1-yl)ethyl)-3-(1-(3-(dimethylamino)propyl)-2-methyl-1H-indol-3-yl)acrylamide

A mixture of sodium (E)-3-(1-(3-(dimethylamino)propyl)-2-methyl-1H-indol-3-yl)acrylate (200 mg, 0.65 mmol), 2-(1H-imidazol-1-yl)ethan-1-amine (72 mg, 0.65 mmol), EDCl (248 mg, 1.35 mmol), HOBt (88 mg, 0.65 mmol) and TEA (136 mg, 1.35 mmol) in DCM (15 mL) was stirred at room temperature overnight. The reaction mixture was washed with saturated Na₂CO₃, dried over Na₂SO₄ and concentrated. The crude product was purified by preparative TLC to give a solid (70 mg, 28%).

¹H NMR (400 MHz, CDCl₃): δ 7.98 (d, J=15.2 Hz, 1H), 7.80 (d, J=7.6 Hz, 1H), 7.51 (br s, 1H), 7.38 (d, J=7.6 Hz, 1H), 7.25-7.15 (m, 2H), 7.10 (br s, 1H), 6.98 (br s, 1H), 6.36 (d, J=15.2 Hz, 1H), 5.93 (t, J=6.0 Hz, 1H), 4.22-4.17 (m, 4H), 3.71 (q, J=6.0 Hz, 2H), 2.57 (s, 3H), 2.28 (t, J=6.8 Hz, 2H), 2.24 (s, 6H), 1.95-1.88 (m, 2H). LCMS: m/z 380.1 [M+H]⁺.

### Preparation of Compound 2068, (E)-3-(1-(3-(dimethylamino)propyl)-2-methyl-1H-indol-3-yl)-N-(2-(pyridin-2-yl)ethyl)acrylamide

A mixture of sodium (E)-3-(1-(3-(dimethylamino)propyl)-2-methyl-1H-indol-3-yl)acrylate (200 mg, 0.65 mmol), 2-(pyridin-2-yl)ethan-1-amine (79 mg, 0.65 mmol), EDCl (248 mg, 1.35 mmol), HOBt (88 mg, 0.65 mmol) and TEA (136 mg, 1.35 mmol) in DCM (15 mL) was stirred at room temperature overnight. The reaction mixture was washed with saturated Na₂CO₃, dried over Na₂SO₄ and concentrated. The crude product was purified by preparative TLC to give a solid (50 mg, 20%).

¹H NMR (400 MHz, CDCl₃): δ 8.59 (d, J=4.8 Hz, 1H), 7.92 (d, J=15.6 Hz, 1H), 7.86 (dd, J=6.8 Hz, 1.6 Hz, 1H), 7.65 (td, J=7.6 Hz, 1.6 Hz, 1H), 7.38 (dd, J=6.8 Hz, 1.2 Hz, 1H), 7.26-7.17 (m, 4H), 6.46 (t, J=5.6 Hz, 1H), 6.39 (d, J=15.6 Hz, 1H), 4.23 (t, J=7.2 Hz, 2H), 3.85 (q, J=6.0 Hz, 1H), 3.11 (t, J=6.4 Hz, 2H), 2.56 (s, 3H), 2.49 (t, J=6.8 Hz, 2H), 2.39 (s, 6H), 2.10-2.03 (m, 2H). LCMS: m/z 391.1 [M+H]⁺.

### Preparation of Compound 2069, (E)-N-(1-benzylpiperidin-4-yl)-3-(1-(3-(dimethylamino)propyl)-2-methyl-1H-indol-3-yl)acrylamide

A mixture of sodium (E)-3-(1-(3-(dimethylamino)propyl)-2-methyl-1H-indol-3-yl)acrylate (200 mg, 0.65 mmol), 1-benzylpiperidin-4-amine (104 mg, 0.65 mmol), EDCl (248 mg, 1.35 mmol), HOBt (88 mg, 0.65 mmol) and TEA (136 mg, 1.35 mmol) in DCM (15 mL) was stirred at room temperature overnight. The reaction mixture was washed with saturated Na₂CO₃, dried over Na₂SO₄ and concentrated. The crude product was purified by preparative TLC to give a solid (130 mg, 44% yield).

¹H NMR (400 MHz, CDCl₃): δ 7.94 (d, J=15.2 Hz, 1H), 7.88 (dd, J=6.8 Hz, 1.6 Hz, 1H), 7.40-7.20 (m, 8H), 6.38 (d, J=15.2 Hz, 1H), 5.46 (d, J=8.0 Hz, 1H), 4.20 (t, J=7.2 Hz, 2H), 4.02 (br s, 1H), 3.58 (s, 2H), 2.92-2.89 (m, 2H), 2.57 (s, 3H), 2.31 (t, J=6.8 Hz, 2H), 2.26-2.22 (m, 8H), 2.06-2.03 (m, 2H), 1.97-1.90 (m, 2H), 1.66-1.56 (m, 2H). LCMS: m/z 459.1 [M+H]⁺.

### Activity of Anti-Tropomyosin Compounds as Monotherapy

In silico modeling has identified binding sites on tropomyosin Tm5NM1, yielding the series of tropomyosin inhibitors the subject of the present invention. Inhibition of Tm5NM1 in tumour cells results in disruption of the actin cytoskeleton and ultimately cell death. The ability of compounds 2001-2012 to disrupt the actin cytoskeleton was assessed in vitro using the microfilament disruption assay. Briefly, cells were seeded (5×10³/well) into 8-well chamber slides (NUNC) and treated with the concentrations of anti-tropomyosin compounds nominated in Table 1 for 24 hours using DMSO as vehicle control. Actin was visualized with Alexa 555 conjugated phalloidin (Molecular probes). Random fields were imaged using an Olympus IX81 microscope. Cells (n>50) were scored on the basis of positive filament staining from n=3 independent experiments.

Cell viability assays were also conducted to assess the anti-proliferative effects of the anti-tropomyosin compounds. Briefly, cells (1×10³/well) were plated (96-well) and treated (48 hr) with anti-tropomyosin drug and viability measured using 3-(4,5-dimethylthiazol-2-yl)-2,5-diphenylterazolium bromide MTT. Cell viability was normalized to control (vehicle alone) and dose-response curves, and half maximal effective concentration (EC₅₀) values were determined using Graph Pad Prism 5 (nonlinear regression sigmoidal dose-response variable slope).

Data demonstrate that those anti-tropomyosin compounds that effectively disrupt the actin microfilament also have a strong anti-proliferative effect on neuroblastoma (SH-EP) and melanoma (SK-MeI-28) cells (Table 1).

TABLE 1
Biological activity of compounds of the invention

| Compound | IC50 (μM) | | Microfilament disruption | | |
|---|---|---|---|---|---|
| ID | SKMEL28 | SHEP | 2.5 μM | 5 μM | 10 μM |
| 2001 | 1.64 | 11.5 | ++ | ++ | +++ |
| 2002 | 2.01 | 9.6 | +++ | +++ | no cells |
| 2003 | 3.1 | 2.3 | ++ | +++ | no cells |
| 2004 | 32.7 | 198 | − | − | − |
| 2005 | 41 | 12.9 | − | + | ++ |
| 2006 | 157 | 12.5 | − | − | + |
| 2007 | 33.1 | 24 | − | + | ++ |
| 2008 | 3.7 | 1.92 | ++ | +++ | no cells |
| 2009 | 1.86 | 2.4 | ++ | +++ | no cells |
| 2010 | 2.75 | 3.2 | − | ++ | +++ |
| 2011 | 12.1 | 13.2 | − | − | + |
| 2012 | 3.2 | 21.7 | − | ++ | +++ |

The ability of compounds 2002, 2009 and 2013-2064 to inhibit the proliferation of cancer cells representative of neuroblastoma, melanoma, prostate cancer, colorectal cancer, non-small cell lung carcinoma, and triple negative breast cancer was assessed. These studies were conducted by contract research (GVK-BIO). Briefly, a pre-determined number of cells as calculated from cell growth assays for each of the cell lines employed were seeded into their respective culture medium (using ATCC culture parameters—http://www.atcc.org) and cultured for 24 h at 37° C. and 5% CO₂ in 96-well culture plates. Once attached, each cell line was then exposed to various concentrations of each respective analogue (30, 10, 3, 1, 0.3 and 0.1 μM), cultured for a further 72 hours and exposed to cell-titre luminescent reagent (100 μL/well) for a further 30 minutes). Luminescence was captured using an EnVision multilabel reader and the data for each analogue concentration compared against no treatment control. Cell viability was normalized to control (vehicle alone) and dose-response curves, and half maximal effective concentration (EC₅₀) values were determined using Graph Pad Prism 6 (nonlinear regression sigmoidal dose-response variable slope).

TABLE 2
Anti-proliferative activity of compounds of the invention against a range of
somatic cancer cells.

| | IC₅₀/μM | | | | | | |
|---|---|---|---|---|---|---|---|
| | | | | | | Lung | Breast |
| Compound | Neuroblastoma | Melanoma | Prostate | | Colorectal | (NSLC) | MDA-MB- |
| ID | SK-N-SH | SK-Mel-28 | DU145 | PC3 | CaCo2 | A549 | 231 |
| 2002 | 12.2 | 5.7 | 11.1 | 22.5 | 23.5 | 11.9 | 11.5 |
| 2009 | 10.8 | 6.5 | 14.3 | 30.0 | 16.1 | 54.3 | 30.0 |
| 2013 | 3.2 | 3.8 | 10.0 | 4.8 | 9.1 | 6.1 | 6.8 |
| 2014 | 3.7 | 3.2 | 7.6 | 6.5 | 9.0 | 25.7 | 6.2 |
| 2015 | 8.8 | 3.9 | 39.2 | 12.4 | 15.9 | 14.1 | 21.1 |
| 2016 | 5.0 | 4.1 | 5.4 | 5.8 | 8.9 | 4.8 | 10.8 |
| 2017 | 17.6 | 8.7 | 15.7 | 11.5 | 12.6 | 13.6 | 18.5 |
| 2018 | 8.5 | 4.3 | 12.1 | 8.0 | 38.0 | 15.9 | 5.7 |
| 2019 | 5.5 | 6.3 | 14.8 | 9.3 | 25.1 | 10.6 | 5.1 |
| 2020 | 3.9 | 3.5 | 5.7 | 5.1 | 4.5 | 3.5 | 5.1 |
| 2021 | 5.4 | 4.3 | 6.9 | 7.2 | 7.4 | 5.4 | 4.8 |
| 2022 | 12.5 | 17.2 | 22.0 | 7.6 | 21.5 | 12.7 | 11.3 |
| 2023 | 3.6 | 2.7 | 4.4 | 5.6 | 10.3 | 5.2 | 4.6 |
| 2024 | 3.4 | 3.0 | 8.4 | 5.7 | 5.3 | 7.7 | 4.1 |
| 2025 | 4.1 | 4.1 | 15.6 | 7.6 | 16.5 | 4.7 | 30.0 |
| 2026 | 2.5 | 1.7 | 9.8 | 5.2 | 4.1 | 3.4 | 4.3 |
| 2027 | 8.2 | 3.8 | 51.9 | 9.3 | 12.3 | 23.5 | 5.6 |
| 2028 | 2.7 | 2.0 | 5.3 | 5.3 | 4.1 | 4.2 | 5.2 |
| 2029 | 2.8 | 5.7 | 4.2 | 2.9 | 4.4 | 4.5 | 3.3 |
| 2030 | 3.6 | 3.4 | 7.9 | 4.0 | 5.4 | 7.9 | 4.3 |
| 2031 | 3.7 | 3.9 | 6.2 | 2.1 | 3.7 | 4.2 | 5.2 |
| 2032 | 4.7 | 4.3 | 7.7 | 6.8 | 5.6 | 9.0 | 4.9 |
| 2033 | 3.3 | 4.2 | 18.6 | 6.8 | 4.2 | 10.8 | 4.5 |
| 2034 | 10.8 | 16.2 | 17.2 | 11.5 | 8.1 | 51.3 | 13.7 |
| 2035 | 3.3 | 3.2 | 6.2 | 5.6 | 5.1 | 8.3 | 3.0 |
| 2036 | 1.5 | 3.2 | 4.7 | 4.9 | 5.1 | 4.3 | 4.4 |
| 2037 | 3.1 | 2.7 | 5.0 | 8.7 | 4.8 | 6.6 | 5.0 |
| 2038 | >30 | >30 | >30 | >30 | >30 | >30 | >30 |
| 2039 | 12.6 | 11.0 | 72.6 | 7.6 | 4.8 | 44.0 | 16.6 |
| 2040 | 26.4 | 16.4 | >30 | >30 | >30 | >30 | >30 |
| 2041 | 13.2 | 17.1 | >30 | >30 | >30 | >30 | >30 |
| 2042 | >30 | >30 | >30 | >30 | >30 | >30 | >30 |
| 2043 | 17.4 | 17.2 | >30 | >30 | >30 | >30 | >30 |
| 2044 | 6.6 | 6.6 | 10.1 | 11.0 | 11.9 | 12.2 | 20.4 |
| 2045 | >30 | >30 | >30 | >30 | >30 | >30 | >30 |

TABLE 3
Anti-proliferative activity of compounds of the invention against a range of
somatic cancer cells.

| | IC₅₀/μM | | | | | | |
|---|---|---|---|---|---|---|---|
| | | | | | | Lung | Breast |
| Compound | Neuroblastoma | Melanoma | Prostate | | Colorectal | (NSLC) | MDA-MB- |
| ID | SK-N-SH | SK-Mel-28 | DU145 | PC3 | CaCo2 | A549 | 231 |
| 2046 | 7.9 | 4.4 | 13.3 | 20.3 | 8.0 | 14.4 | 11.3 |
| 2047 | 10.9 | 13.5 | 13.6 | 14.9 | 8.8 | 14.3 | 12.0 |
| 2048 | >30 | >30 | >30 | >30 | >30 | >30 | >30 |
| 2049 | 8.9 | 8.7 | >30 | 8.6 | >30 | >30 | 7.5 |
| 2050 | 21.4 | 5.3 | 15.9 | 26.4 | 10.4 | 19.8 | 14.0 |
| 2051 | 10.9 | 6.0 | >30 | >30 | >30 | >30 | 10.3 |
| 2052 | 10.9 | 5.7 | 9.2 | 10.9 | 16.0 | 11.8 | 12.7 |
| 2053 | 4.8 | 4.7 | 13.6 | 11.0 | 12.2 | 13.5 | 12.6 |
| 2054 | 10.4 | 5.4 | >30 | 6.9 | >30 | 17.8 | 11.8 |
| 2055 | >30 | 1.1 | >30 | >30 | >30 | >30 | >30 |
| 2056 | 11.2 | 4.8 | 18.9 | 10.8 | 10.1 | 14.7 | 13.9 |
| 2057 | 7.2 | 4.4 | 30.0 | 9.0 | 13.3 | 21.1 | 7.5 |
| 2058 | 8.0 | 4.8 | 21.1 | 12.0 | 10.8 | 13.9 | 29.8 |
| 2059 | 4.2 | 3.8 | 10.1 | 9.2 | 19.6 | 10.2 | 7.0 |
| 2060 | 10.8 | 7.5 | >30 | >30 | >30 | >30 | 6.1 |
| 2061 | >30 | 8.7 | >30 | >30 | >30 | >30 | >30 |
| 2062 | 20.6 | 9.0 | >30 | 13.1 | 18.6 | >30 | 8.1 |
| 2063 | 5.3 | 3.5 | 13.0 | 7.2 | 4.1 | 10.9 | 3.0 |
| 2064 | 4.3 | 3.5 | 14.0 | 6.9 | 29.1 | 7.8 | 5.8 |

In order to demonstrate that the compounds of the invention impaired Tm5NM1 function the impact of compound 2026 on Tm5NM1-regulated actin filament depolymerization was assessed using a well-characterized pyrene-based actin filament depolymerization assay (Broschat, 1990; Kostyukova and Hitchcock, 2004). A brief overview and rationale of the assay is as follows: to promote depolymerization, pyrene-labelled actin filaments were diluted below the critical concentration of the pointed end (0.5 μM, as defined by Pollard et al., 1986). A decline in fluorescence was measured over time as actin monomers dissociate. It is well established that in the presence of Tm5NM1 the rate of actin depolymerization is significantly reduced (Bonello 2013). Therefore, any compound, which interacts with, and impacts Tm5NM1 function, would nullify the protective effect of Tm5NM1 on actin depolymerization.

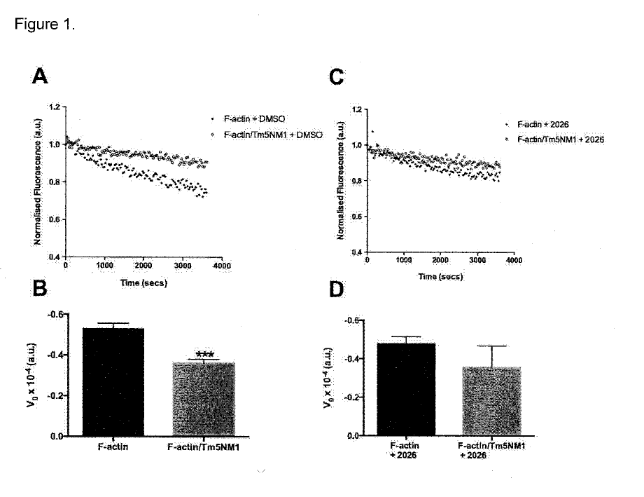

For all assays the depolymerization of F-actin alone and F-actin-coated with the human homologue of Tm5NM1 was used as a comparative control. Briefly, Tm5NM1 was pre-incubated with F-actin for 20 min prior to diluting the filaments, to allow for proper assembly of the Tm5NM1 polymer. As expected, in the presence of saturating amounts of Tm5NM1, the initial rate (V₀) of F-actin depolymerization was significantly slower for Tm5NM1-containing actin filaments (−0.36±0.02×10−4) when compared to actin filaments alone (−0.53±0.027×10−4; FIGS. 1A and B, p<0.0001).

The depolymerization of F-actin alone and F-actin-coated with Tm5NM1 was then measured in the presence of compound and initial rates of depolymerization were compared. Tm5NM1 was pre-incubated with 50 μM compound 2026 prior to being added to the actin filaments as previously described. In the presence of compound 2026, Tm5NM1-containing actin filaments depolymerized at a similar rate to F-actin alone (−0.35±0.046×10−4 vs −0.47±0.039×10−4; FIGS. 1C and D, p=0.1) demonstrating that compound 2026 interacts with and impairs Tm5NM1 function.

### SELECTED REFERENCE ARTICLES

Broschat, K. O. (1990). Tropomyosin prevents depolymerization of actin filaments from the pointed end. J Biol Chem 265, 21323-21329.
Kostyukova, A. S., and Hitchcock-DeGregori, S. E. (2004). Effect of the structure of the N terminus of tropomyosin on tropomodulin function. J Biol Chem 279, 5066-5071.
Pollard, T. D. (1986). Rate constants for the reactions of ATP- and ADP-actin with the ends of actin filaments. J Cell Biol 103, 2747-2754.
Bonello, T. B (2013). Characterising the impact of tropomyosin targeting compounds in the actin cytoskeleton. Ph.D thesis, School of Medical Sciences, University of New South Wales, Australia

1. A compound of formula (I) or a pharmaceutically acceptable drug or prodrug thereof, wherein: 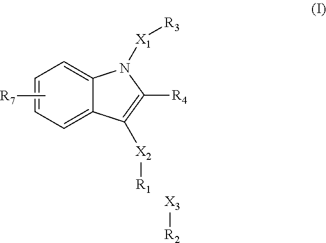
R₁═(CH₂)₀₋₅, 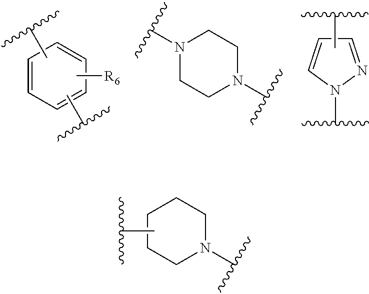
X₁═(CH₂)₀₋₅
X₂ and X₃═O, NH, NHR₅, C(O), C(O)NH, (CH₂)₀₋₅, C(R₅)C(R_{4′})C(O), C(R₅)C(R_{4′})C(O)NH, pyrazole, isooxazole, (R_{4′})pyrimidine
X₄═H, O, NH, NR₅
R₂=CH₃ 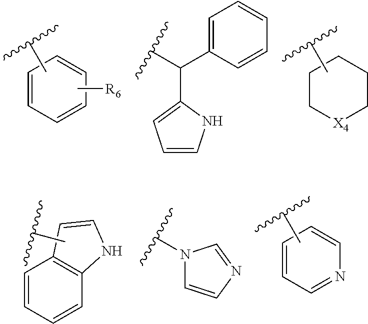
R₄ and R_{4′}═H, CH₃
R₅═H, CH₃, (CH₂)₁₋₅CH₃, (CH₂)₁₋₅OCH₃, CF₃, CN, OCF₃
R₆═H, alkyl, halo, alkoxy, amino, aminoalkyl, diaminoalkyl, or a dioxolane ring fused to 2 adjacent carbon atoms of R₁ or R₂
R₇═H, alkyl, alkoxy
R₃═NH₂, N(R₅)₂,

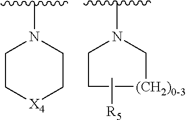

2. A compound according to claim 1, wherein X₁ is (CH₂)₃.

3. A compound according to claim 1, wherein R₃ is N(R₅)₂.

4. A compound according to claim 3, wherein R₅ is CH₃.

5. A compound according to claim 3, wherein R₅ is CH₂CH₃.

6. A compound according to claim 1 or 2, wherein R₃ is

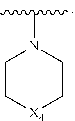

7. A compound according to claim 6, wherein X₄ is NR₅.

8. A compound according to claim 7, wherein R₅ is CH₃.

9. A compound according to claim 6, wherein X₄ is O.

10. A compound according to claim 1, wherein R₄ is CH₃.

11. A compound according to claim 1, wherein X₂ is C(R₅)C(R_{4′})C(O), (R_{4′}) pyrimidine, C(O), or C(R₅)C(R_{4′})C(O)NH.

12. A compound according to claim 11, wherein X₂ is C(R₅)C(R_{4′})C(O), R₅ is H and R_{4′} is H.

13. A compound according to claim 11, wherein X₂ is C(R₅)C(R_{4′})C(O), R₅ is H and R_{4′} is CH₃.

14. A compound according to claim 11, wherein X₂ is (R_{4′}) pyrimidine and R_{4′} is CH₃.

15. A compound according to claim 11, wherein X₂ is C(R₅)C(R_{4′})C(O)NH, R₅ is H and R_{4′} is H.

16. A compound according to claim 1, wherein R₁ is CH₂,

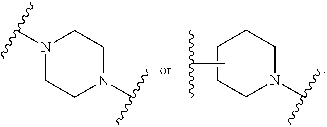

17. A compound according to claim 16, wherein

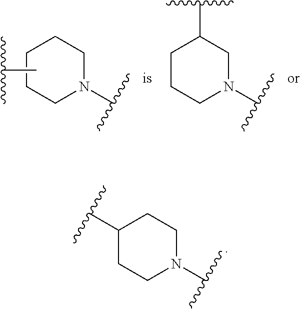

18. A compound according to claim 1, wherein X₃ is CH₂, (CH₂)₂, (CH₂)₃ or C(O).

19. A compound according to claim 1, wherein R₂ is

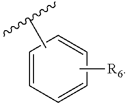

20. A compound according to claim 19, wherein R₆ is H, alkoxy, halo or the dioxolane ring.

21. A compound according to claim 20, wherein alkoxy is OCH₃.

22. A compound according to claim 20, wherein halo is F.

23. A compound according to claim 1, wherein R₂ is CH₃.

24. A compound according to claim 1, wherein R₂ is

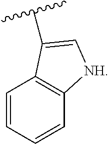 [SMILES: *c1c[nH]c2ccccc12]

25. A compound according to claim 1, wherein R₂ is

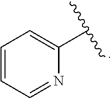 [SMILES: *c1ccccn1]

26. A compound according to claim 1, wherein R₇ is H.

27. A compound according to claim 1, wherein R₇ is alkoxy.

28. A compound according to claim 27, wherein alkoxy is OCH₂CH₃ or OCH₃.

29. A compound according to claim 1 selected from the group consisting of:

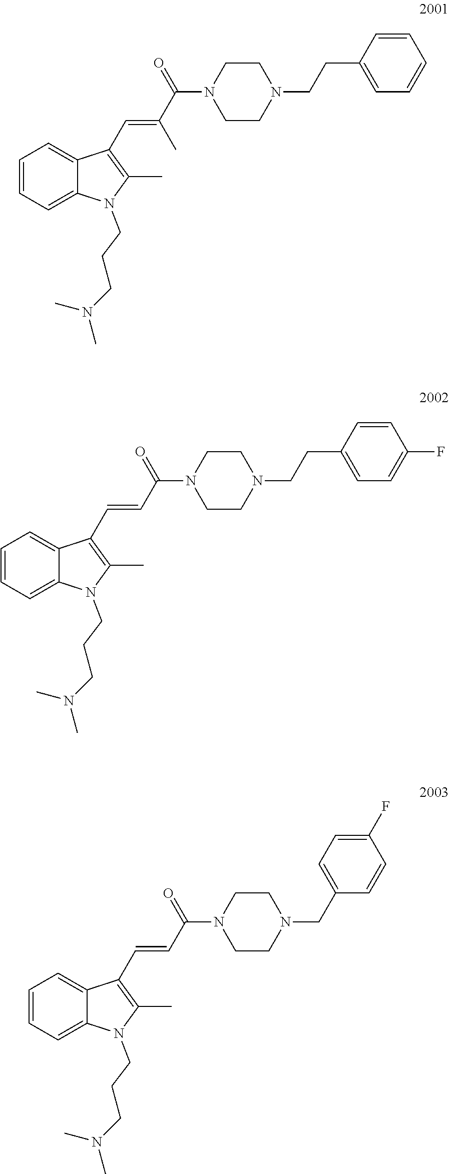 [SMILES: C/C(=C\c1c(C)n(CCCN(C)C)c2ccccc12)C(=O)N1CCN(CCc2ccccc2)CC1 ; Cc1c(/C=C/C(=O)N2CCN(CCc3ccc(F)cc3)CC2)c2ccccc2n1CCCN(C)C ; Cc1c(/C=C/C(=O)N2CCN(Cc3ccc(F)cc3)CC2)c2ccccc2n1CCCN(C)C]

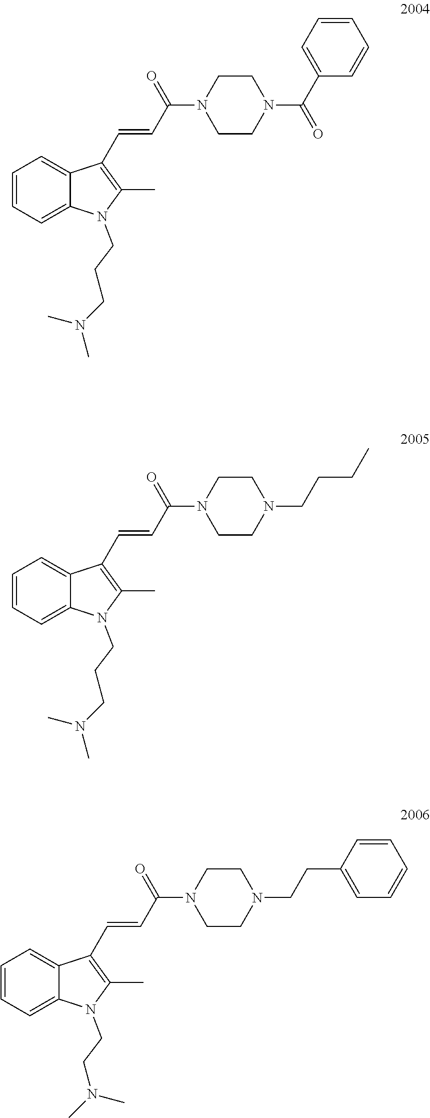 [SMILES: Cc1c(/C=C/C(=O)N2CCN(C(=O)c3ccccc3)CC2)c2ccccc2n1CCCN(C)C ; CCCCN1CCN(C(=O)/C=C/c2c(C)n(CCCN(C)C)c3ccccc23)CC1 ; Cc1c(/C=C/C(=O)N2CCN(CCc3ccccc3)CC2)c2ccccc2n1CCN(C)C]

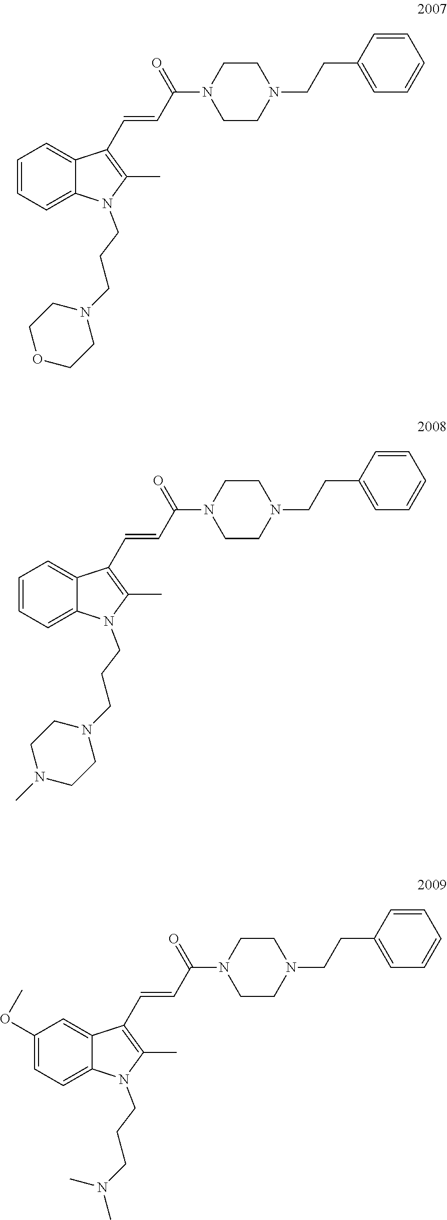 [SMILES: Cc1c(/C=C/C(=O)N2CCN(CCc3ccccc3)CC2)c2ccccc2n1CCCN1CCOCC1 ; Cc1c(/C=C/C(=O)N2CCN(CCc3ccccc3)CC2)c2ccccc2n1CCCN1CCN(C)CC1 ; COc1ccc2c(c1)c(/C=C/C(=O)N1CCN(CCc3ccccc3)CC1)c(C)n2CCCN(C)C]

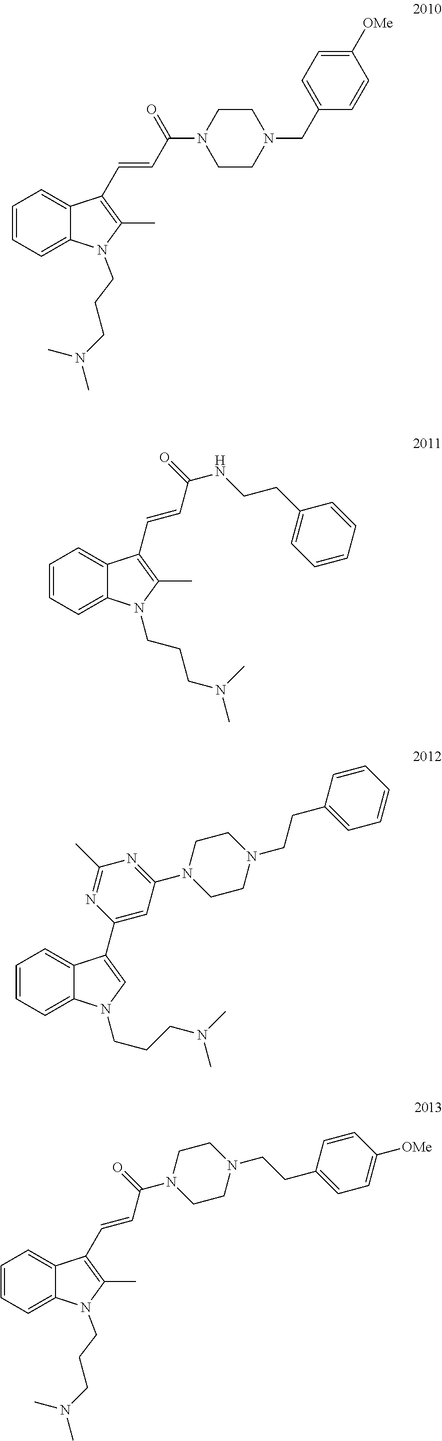 [SMILES: COc1ccc(CN2CCN(C(=O)/C=C/c3c(C)n(CCCN(C)C)c4ccccc34)CC2)cc1 ; Cc1c(/C=C/C(=O)NCCc2ccccc2)c2ccccc2n1CCCN(C)C ; Cc1nc(-c2cn(CCCN(C)C)c3ccccc23)cc(N2CCN(CCc3ccccc3)CC2)n1 ; COc1ccc(CCN2CCN(C(=O)/C=C/c3c(C)n(CCCN(C)C)c4ccccc34)CC2)cc1]

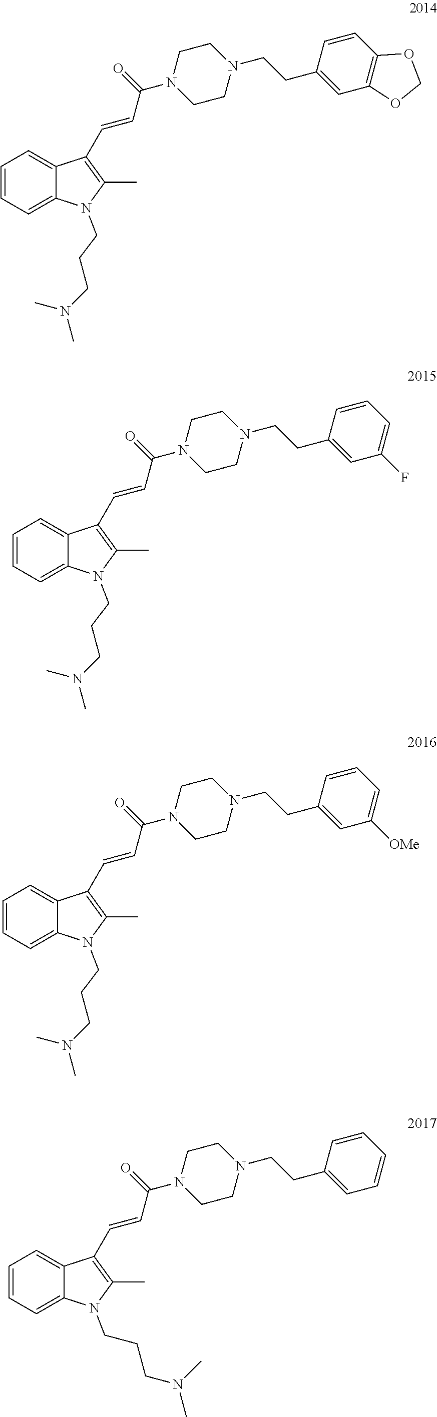 [SMILES: Cc1c(/C=C/C(=O)N2CCN(CCc3ccc4c(c3)OCO4)CC2)c2ccccc2n1CCCN(C)C ; Cc1c(/C=C/C(=O)N2CCN(CCc3cccc(F)c3)CC2)c2ccccc2n1CCCN(C)C ; COc1cccc(CCN2CCN(C(=O)/C=C/c3c(C)n(CCCN(C)C)c4ccccc34)CC2)c1 ; Cc1c(/C=C/C(=O)N2CCN(CCc3ccccc3)CC2)c2ccccc2n1CCCN(C)C]

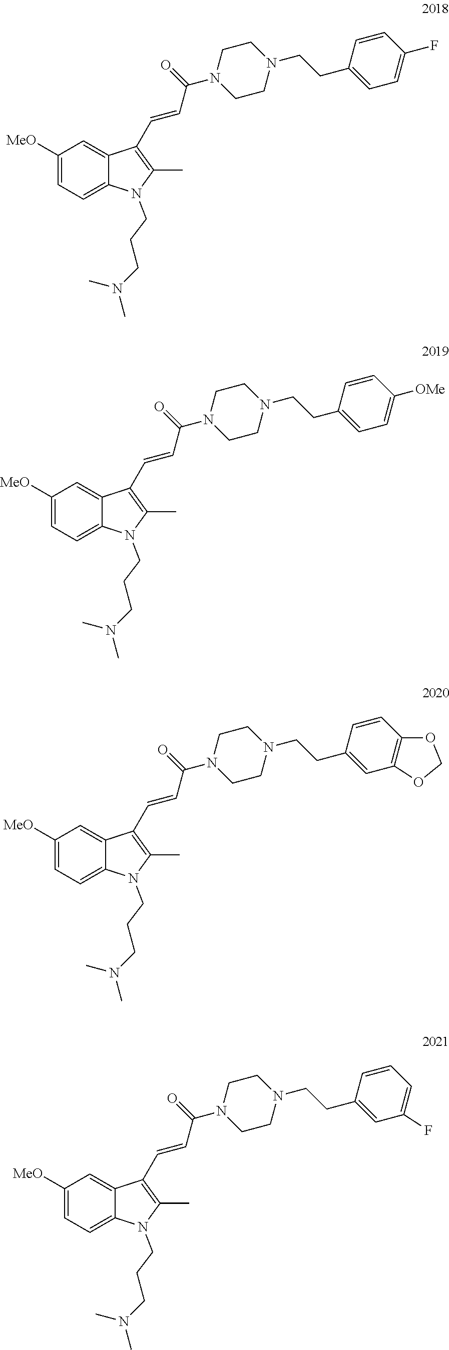 [SMILES: COc1ccc2c(c1)c(/C=C/C(=O)N1CCN(CCc3ccc(F)cc3)CC1)c(C)n2CCCN(C)C ; COc1ccc(CCN2CCN(C(=O)/C=C/c3c(C)n(CCCN(C)C)c4ccc(OC)cc34)CC2)cc1 ; COc1ccc2c(c1)c(/C=C/C(=O)N1CCN(CCc3ccc4c(c3)OCO4)CC1)c(C)n2CCCN(C)C ; COc1ccc2c(c1)c(/C=C/C(=O)N1CCN(CCc3cccc(F)c3)CC1)c(C)n2CCCN(C)C]

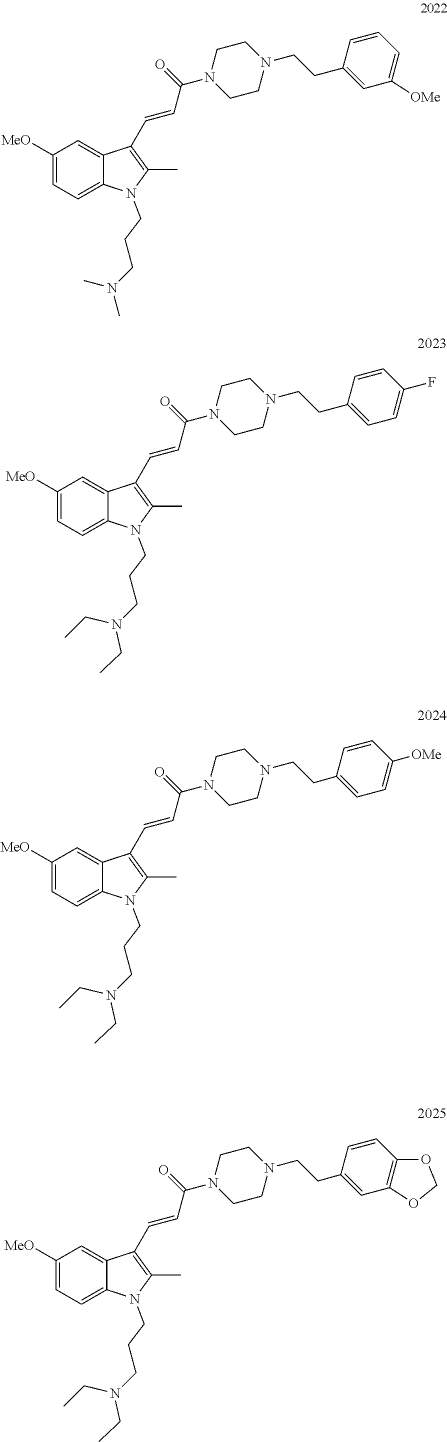 [SMILES: COc1cccc(CCN2CCN(C(=O)/C=C/c3c(C)n(CCCN(C)C)c4ccc(OC)cc34)CC2)c1 ; CCN(CC)CCCn1c(C)c(/C=C/C(=O)N2CCN(CCc3ccc(F)cc3)CC2)c2cc(OC)ccc21 ; CCN(CC)CCCn1c(C)c(/C=C/C(=O)N2CCN(CCc3ccc(OC)cc3)CC2)c2cc(OC)ccc21 ; CCN(CC)CCCn1c(C)c(/C=C/C(=O)N2CCN(CCc3ccc4c(c3)OCO4)CC2)c2cc(OC)ccc21]

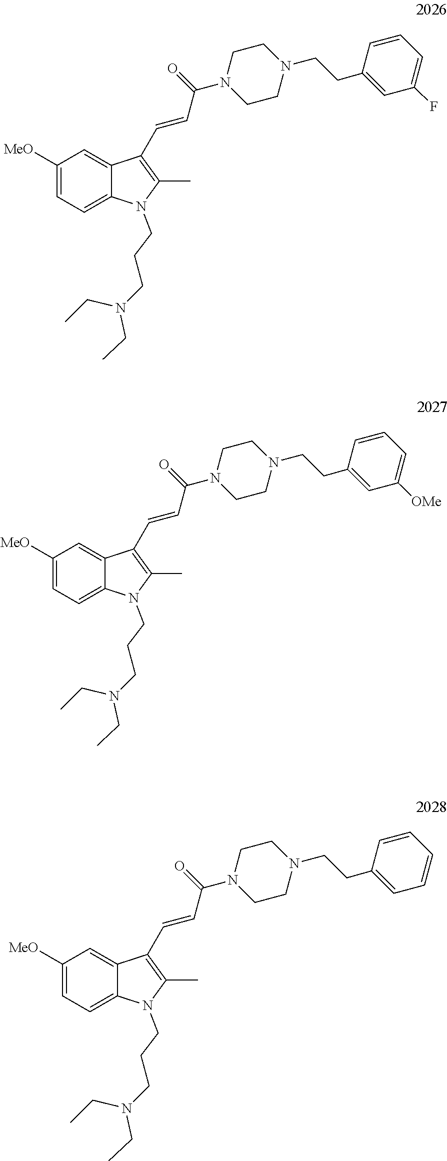 [SMILES: CCN(CC)CCCn1c(C)c(/C=C/C(=O)N2CCN(CCc3cccc(F)c3)CC2)c2cc(OC)ccc21 ; CCN(CC)CCCn1c(C)c(/C=C/C(=O)N2CCN(CCc3cccc(OC)c3)CC2)c2cc(OC)ccc21 ; CCN(CC)CCCn1c(C)c(/C=C/C(=O)N2CCN(CCc3ccccc3)CC2)c2cc(OC)ccc21]

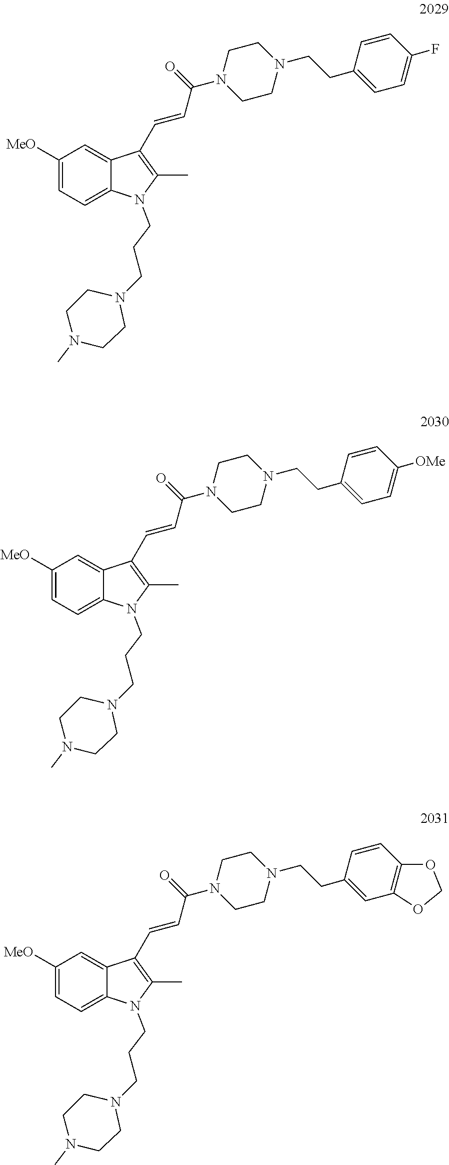 [SMILES: COc1ccc2c(c1)c(/C=C/C(=O)N1CCN(CCc3ccc(F)cc3)CC1)c(C)n2CCCN1CCN(C)CC1 ; COc1ccc(CCN2CCN(C(=O)/C=C/c3c(C)n(CCCN4CCN(C)CC4)c4ccc(OC)cc34)CC2)cc1 ; COc1ccc2c(c1)c(/C=C/C(=O)N1CCN(CCc3ccc4c(c3)OCO4)CC1)c(C)n2CCCN1CCN(C)CC1]

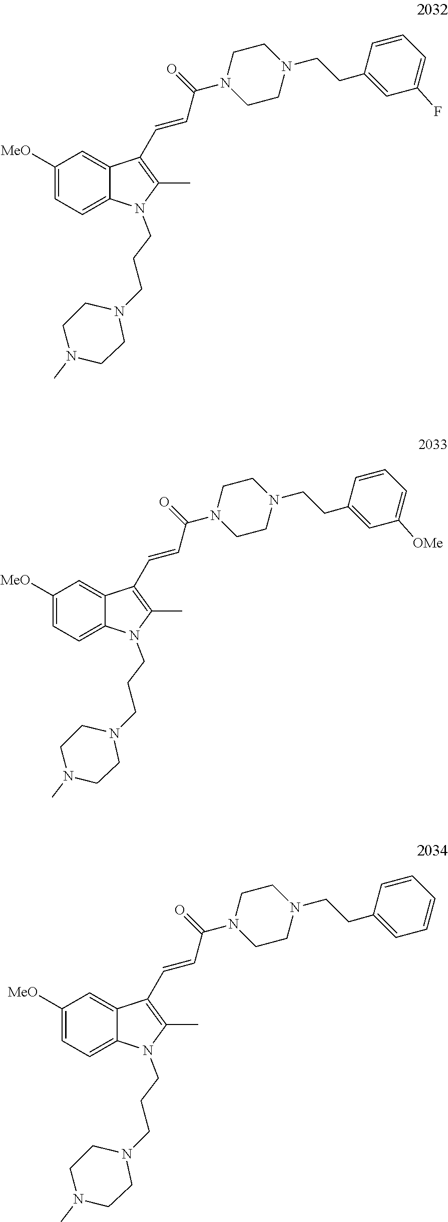 [SMILES: COc1ccc2c(c1)c(/C=C/C(=O)N1CCN(CCc3cccc(F)c3)CC1)c(C)n2CCCN1CCN(C)CC1 ; COc1cccc(CCN2CCN(C(=O)/C=C/c3c(C)n(CCCN4CCN(C)CC4)c4ccc(OC)cc34)CC2)c1 ; COc1ccc2c(c1)c(/C=C/C(=O)N1CCN(CCc3ccccc3)CC1)c(C)n2CCCN1CCN(C)CC1]

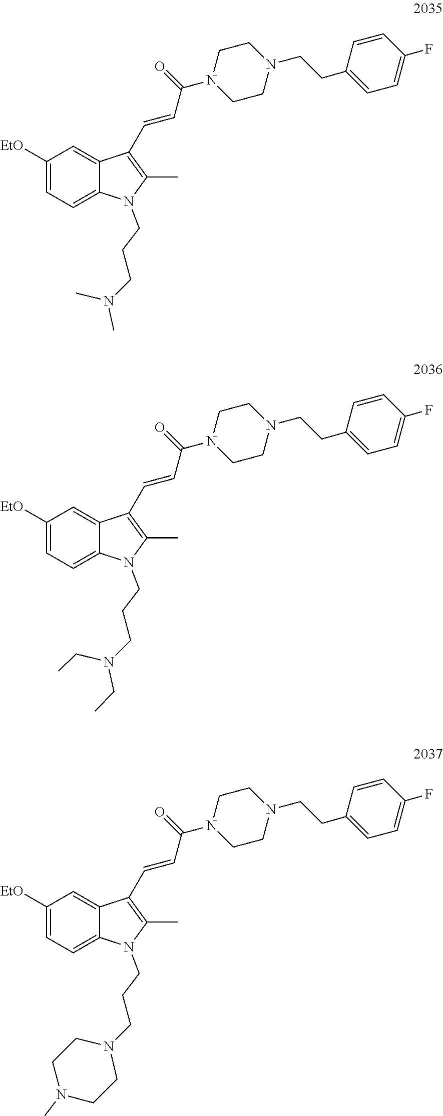 [SMILES: CCOc1ccc2c(c1)c(/C=C/C(=O)N1CCN(CCc3ccc(F)cc3)CC1)c(C)n2CCCN(C)C ; CCOc1ccc2c(c1)c(/C=C/C(=O)N1CCN(CCc3ccc(F)cc3)CC1)c(C)n2CCCN(CC)CC ; CCOc1ccc2c(c1)c(/C=C/C(=O)N1CCN(CCc3ccc(F)cc3)CC1)c(C)n2CCCN1CCN(C)CC1]

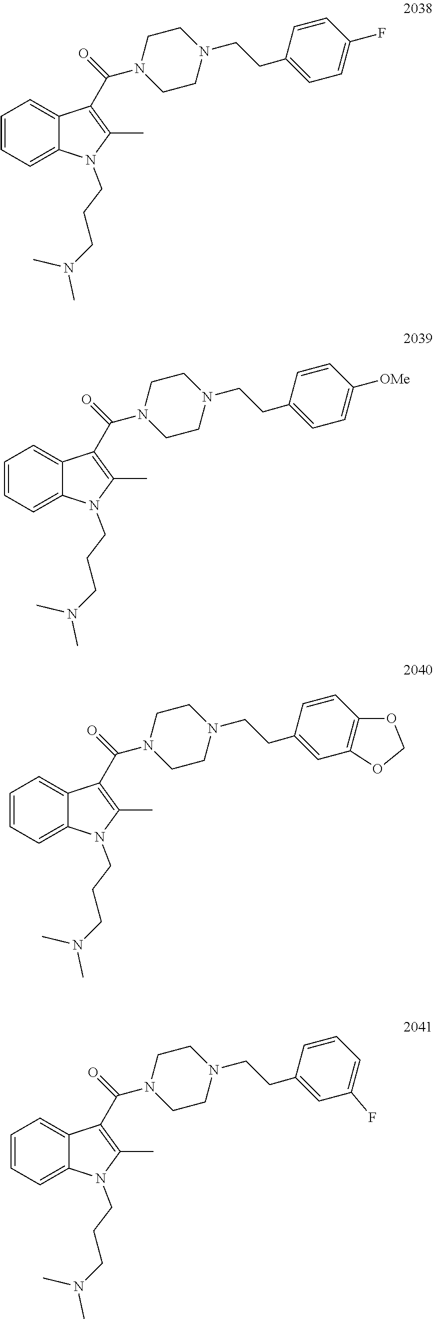 [SMILES: Cc1c(C(=O)N2CCN(CCc3ccc(F)cc3)CC2)c2ccccc2n1CCCN(C)C ; COc1ccc(CCN2CCN(C(=O)c3c(C)n(CCCN(C)C)c4ccccc34)CC2)cc1 ; Cc1c(C(=O)N2CCN(CCc3ccc4c(c3)OCO4)CC2)c2ccccc2n1CCCN(C)C ; Cc1c(C(=O)N2CCN(CCc3cccc(F)c3)CC2)c2ccccc2n1CCCN(C)C]

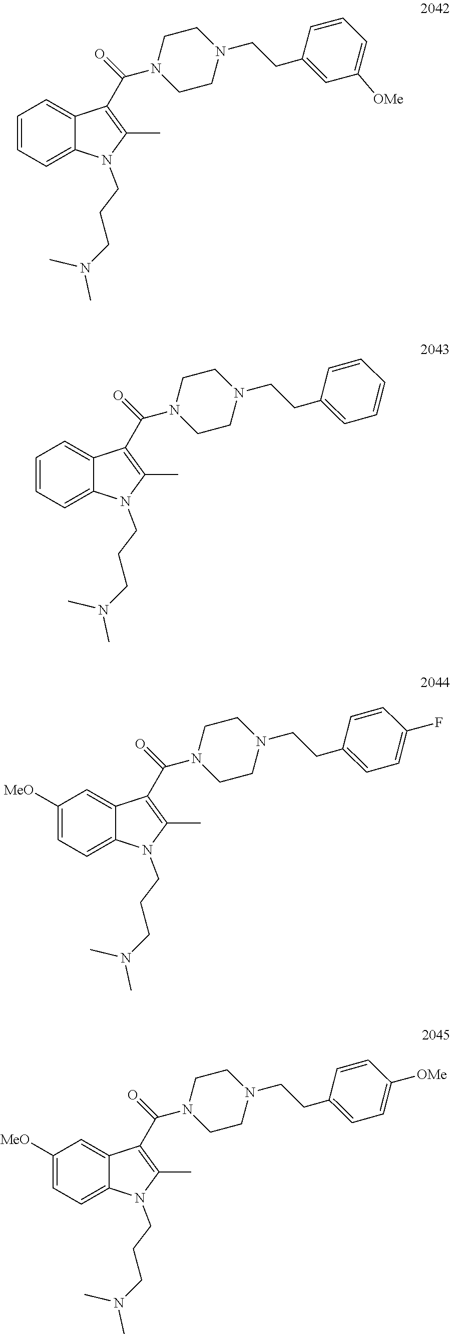 [SMILES: COc1cccc(CCN2CCN(C(=O)c3c(C)n(CCCN(C)C)c4ccccc34)CC2)c1 ; Cc1c(C(=O)N2CCN(CCc3ccccc3)CC2)c2ccccc2n1CCCN(C)C ; COc1ccc2c(c1)c(C(=O)N1CCN(CCc3ccc(F)cc3)CC1)c(C)n2CCCN(C)C ; COc1ccc(CCN2CCN(C(=O)c3c(C)n(CCCN(C)C)c4ccc(OC)cc34)CC2)cc1]

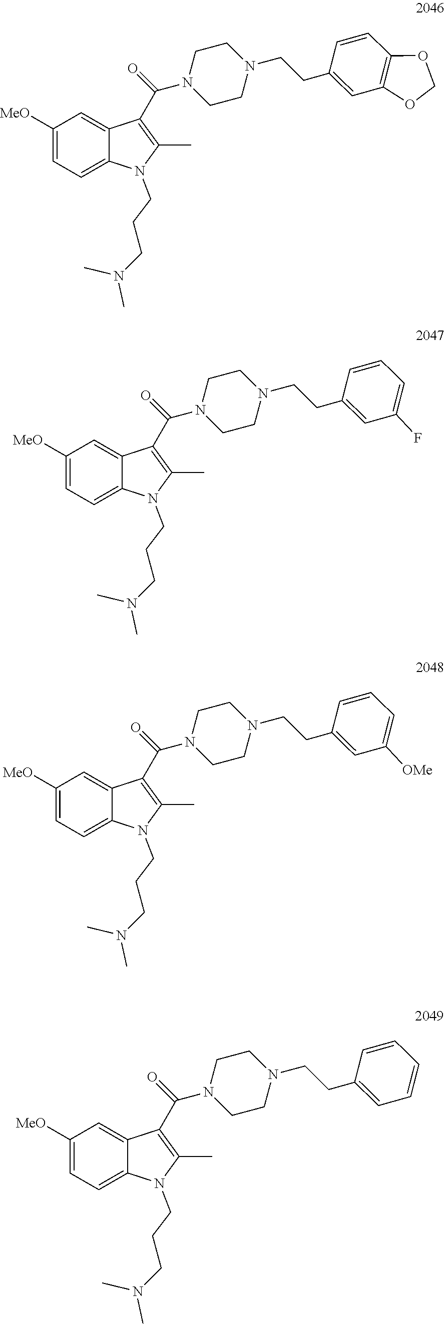 [SMILES: COc1ccc2c(c1)c(C(=O)N1CCN(CCc3ccc4c(c3)OCO4)CC1)c(C)n2CCCN(C)C ; COc1ccc2c(c1)c(C(=O)N1CCN(CCc3cccc(F)c3)CC1)c(C)n2CCCN(C)C ; COc1cccc(CCN2CCN(C(=O)c3c(C)n(CCCN(C)C)c4ccc(OC)cc34)CC2)c1 ; COc1ccc2c(c1)c(C(=O)N1CCN(CCc3ccccc3)CC1)c(C)n2CCCN(C)C]

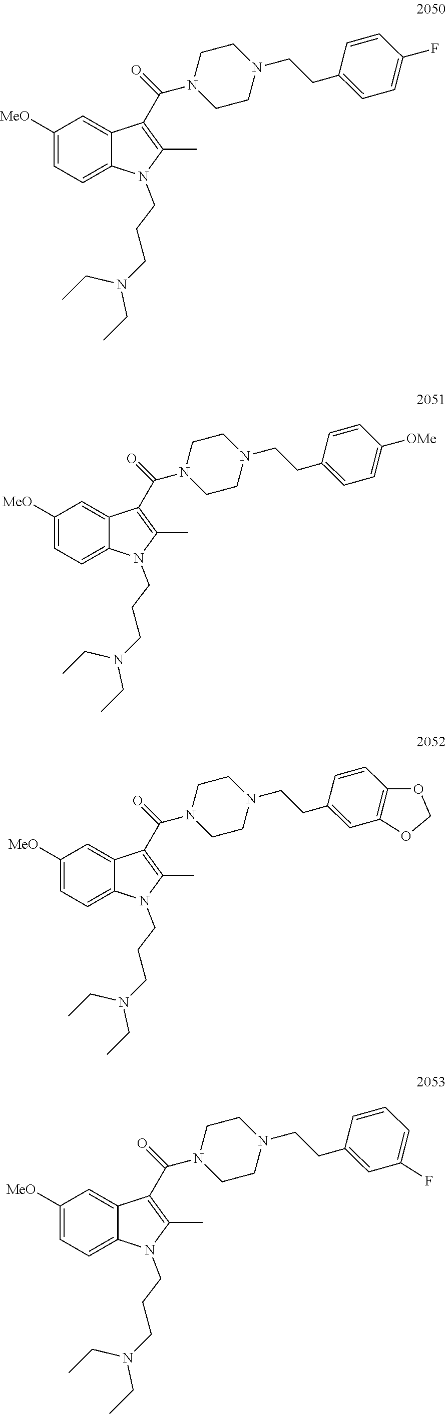 [SMILES: CCN(CC)CCCn1c(C)c(C(=O)N2CCN(CCc3ccc(F)cc3)CC2)c2cc(OC)ccc21 ; CCN(CC)CCCn1c(C)c(C(=O)N2CCN(CCc3ccc(OC)cc3)CC2)c2cc(OC)ccc21 ; CCN(CC)CCCn1c(C)c(C(=O)N2CCN(CCc3ccc4c(c3)OCO4)CC2)c2cc(OC)ccc21 ; CCN(CC)CCCn1c(C)c(C(=O)N2CCN(CCc3cccc(F)c3)CC2)c2cc(OC)ccc21]

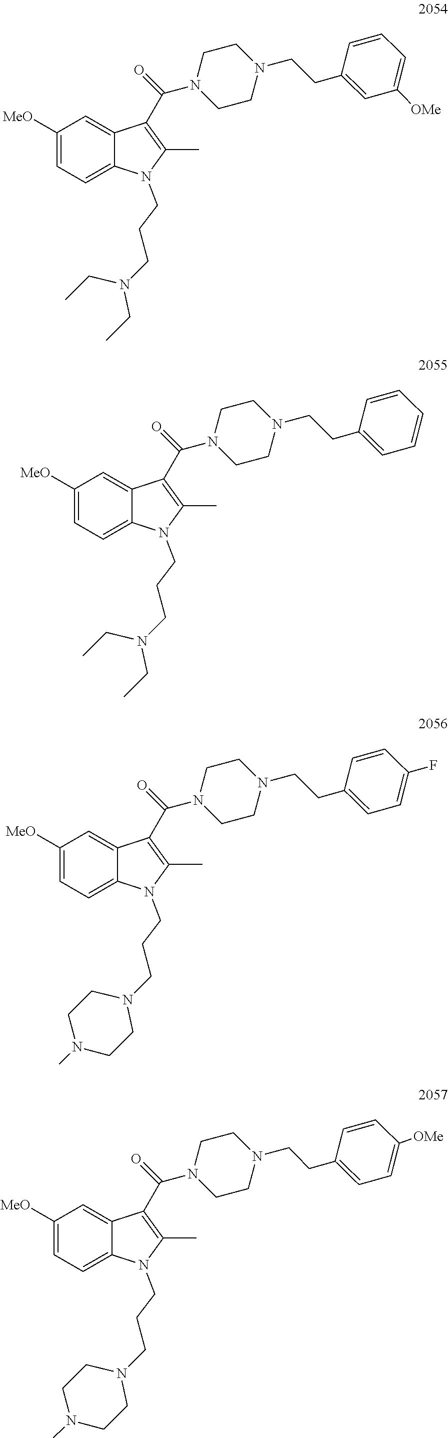 [SMILES: CCN(CC)CCCn1c(C)c(C(=O)N2CCN(CCc3cccc(OC)c3)CC2)c2cc(OC)ccc21 ; CCN(CC)CCCn1c(C)c(C(=O)N2CCN(CCc3ccccc3)CC2)c2cc(OC)ccc21 ; COc1ccc2c(c1)c(C(=O)N1CCN(CCc3ccc(F)cc3)CC1)c(C)n2CCCN1CCN(C)CC1 ; COc1ccc(CCN2CCN(C(=O)c3c(C)n(CCCN4CCN(C)CC4)c4ccc(OC)cc34)CC2)cc1]

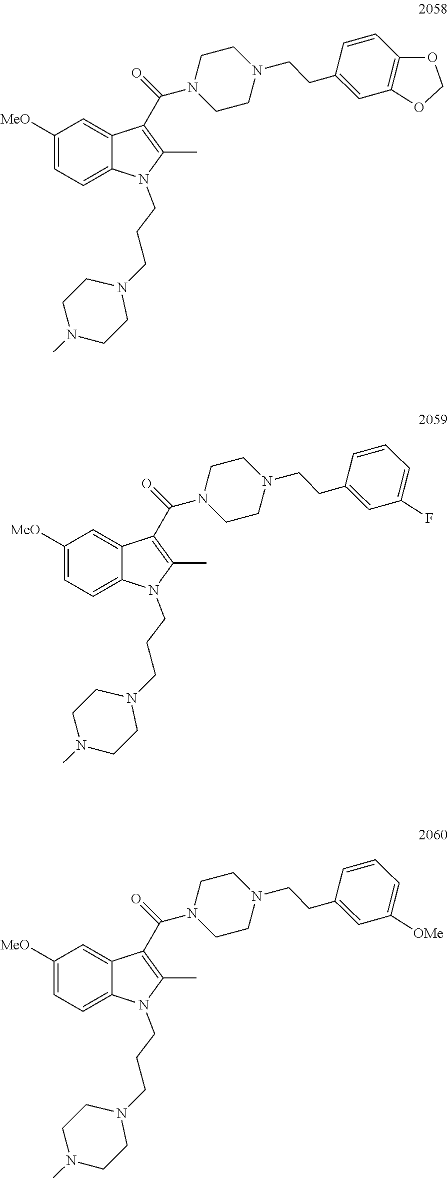 [SMILES: COc1ccc2c(c1)c(C(=O)N1CCN(CCc3ccc4c(c3)OCO4)CC1)c(C)n2CCCN1CCN(C)CC1 ; COc1ccc2c(c1)c(C(=O)N1CCN(CCc3cccc(F)c3)CC1)c(C)n2CCCN1CCN(C)CC1 ; COc1cccc(CCN2CCN(C(=O)c3c(C)n(CCCN4CCN(C)CC4)c4ccc(OC)cc34)CC2)c1]

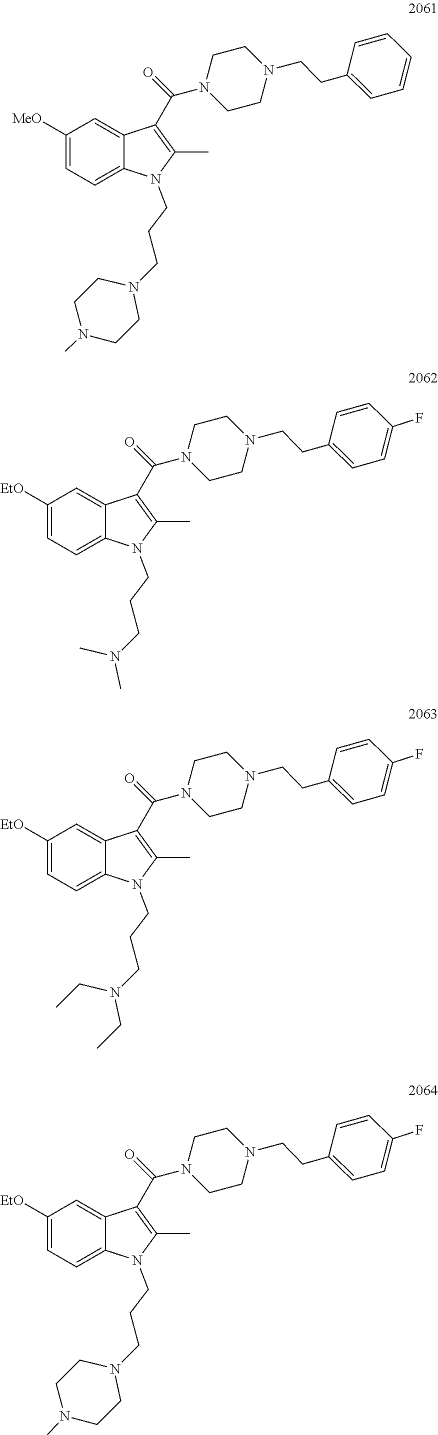 [SMILES: COc1ccc2c(c1)c(C(=O)N1CCN(CCc3ccccc3)CC1)c(C)n2CCCN1CCN(C)CC1 ; CCOc1ccc2c(c1)c(C(=O)N1CCN(CCc3ccc(F)cc3)CC1)c(C)n2CCCN(C)C ; CCOc1ccc2c(c1)c(C(=O)N1CCN(CCc3ccc(F)cc3)CC1)c(C)n2CCCN(CC)CC ; CCOc1ccc2c(c1)c(C(=O)N1CCN(CCc3ccc(F)cc3)CC1)c(C)n2CCCN1CCN(C)CC1]

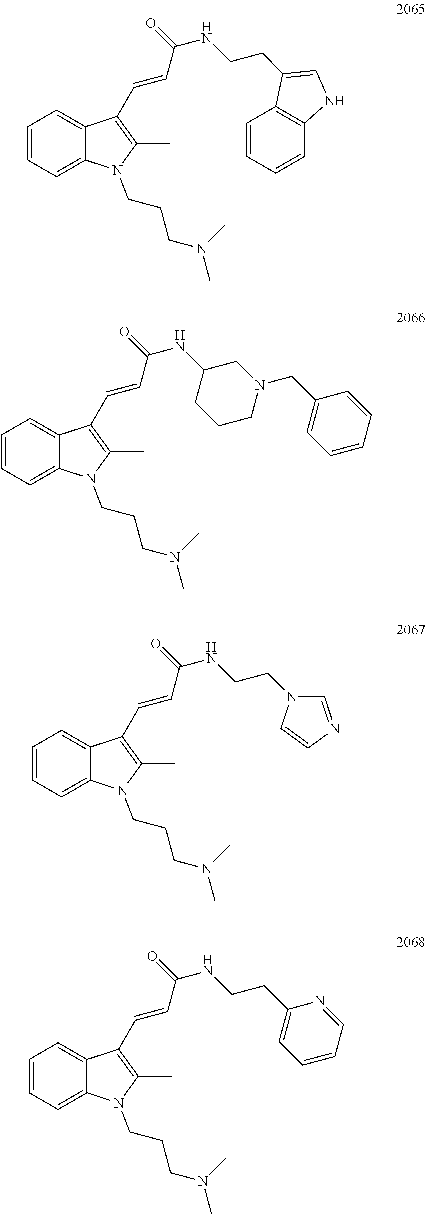 [SMILES: Cc1c(/C=C/C(=O)NCCc2c[nH]c3ccccc23)c2ccccc2n1CCCN(C)C ; Cc1c(/C=C/C(=O)NC2CCCN(Cc3ccccc3)C2)c2ccccc2n1CCCN(C)C ; Cc1c(/C=C/C(=O)NCCn2ccnc2)c2ccccc2n1CCCN(C)C ; Cc1c(/C=C/C(=O)NCCc2ccccn2)c2ccccc2n1CCCN(C)C]

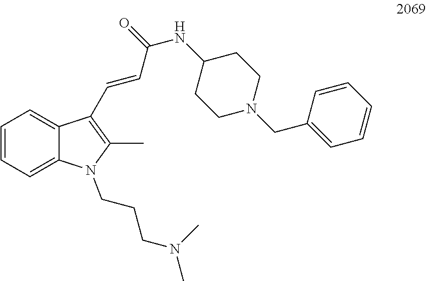 [SMILES: Cc1c(/C=C/C(=O)NC2CCN(Cc3ccccc3)CC2)c2ccccc2n1CCCN(C)C]

30. A pharmaceutical composition comprising a compound according to claim 1.

31. A method a proliferative disease in a subject in need thereof, the method comprising administering to the subject a therapeutically effective amount of a compound according to claim 1.

32.-33. (canceled)

34. A method according to claim 31, wherein the proliferative disease is cancer.

35. (canceled)

36. A method for preventing recurrence of a solid tumor in a subject, the method comprising administering to the subject a compound according to claim 1.

37.-38. (canceled)
